(19) United States
(12) Patent Application Publication
(10) Pub. No.: US 2010/0099561 A1
(43) Pub. Date: Apr. 22, 2010

(54) HETEROBICYCLIC ALKYLTHIO-BRIDGED ISOXAZOLINES

(75) Inventors: THOMAS PAUL SELBY, Hockessin, DE (US); BRENTON TODD SMITH, Exton, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/578,642

(22) Filed: Oct. 14, 2009

Related U.S. Application Data
(60) Provisional application No. 61/105,546, filed on Oct. 15, 2008.

(51) Int. Cl. A01N 43/90; C07D 413/12; C07D 498/04; C07D 517/04; C07D 471/04; C07D 487/04; A01N 43/80; A01P 13/00

(52) U.S. Cl. 504/103; 546/157; 544/354; 544/278; 544/91; 544/48; 544/282; 544/350; 544/236; 504/247; 504/235; 504/241; 504/223; 504/221; 504/237; 504/131; 504/136; 504/137; 504/130

(57) ABSTRACT

Disclosed are compounds of Formula 1, N-oxides, and salts thereof,

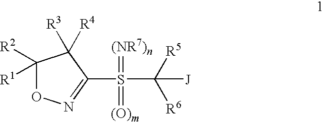

wherein
J is

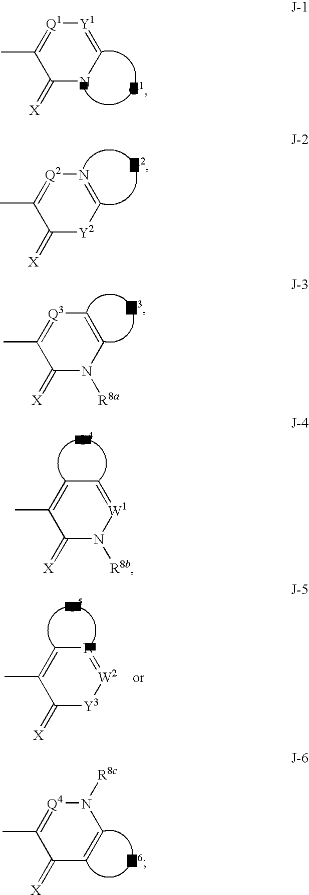

and R¹, R², R³, R⁴, R⁵, R⁶, R⁷, R^{8a}, R^{8b}, R^{8c}, G¹, G², G³, G⁴, G⁵, G⁶, Q¹, Q², Q³, Q⁴, W¹, W², Y¹, Y², Y³, m and n are as defined in the disclosure.
   Also disclosed are compositions containing the compounds of Formula 1 and methods for controlling undesired vegetation comprising contacting the undesired vegetation or its environment with an effective amount of a compound or a composition of the invention.

### FIELD OF THE INVENTION

This invention relates to certain heterobicyclic alkylthio-bridged isoxazoline compounds, their N-oxides, salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safer or have different sites of action.

### SUMMARY OF THE INVENTION

This invention is directed to compounds of Formula 1 (including all geometric and stereoisomers), N-oxides, and salts thereof, agricultural compositions containing them and their use as herbicides or plant growth regulators:

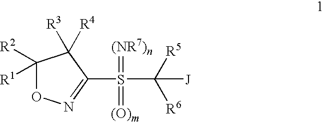

wherein

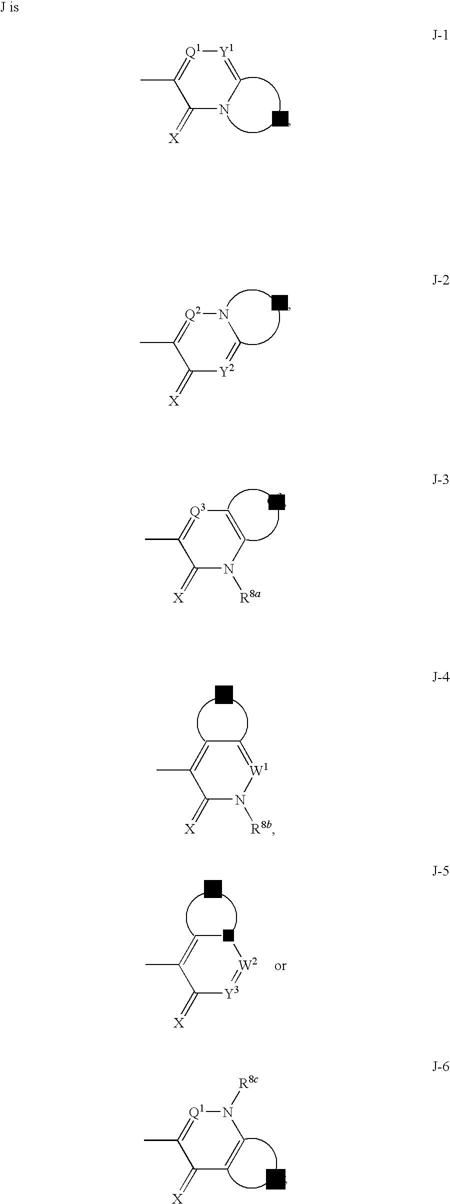

Q¹ is N or CR^{9a};
Q² is N or CR^{9b};
Q³ is N or CR^{9c};
Q⁴ is N or CR^{9d};
W¹ is N or CR^{10a};
W² is N or CR^{10b};
Y¹ is N or CR^{11a};
Y² is N or CR^{11b};
Y³ is N or CR^{11c};
X is O or S;
each G¹, G², G³, G⁴, G⁵ and G⁶ taken together with the two ring fusion atoms to which each is bonded, independently forms a fused 5-, 6- or 7-membered ring containing ring members selected from carbon atoms and up to 4 heteroatoms independently selected from up to 2 oxygen, up to 2 sulfur and up to 3 nitrogen atoms, wherein up to 1 carbon atom ring member is selected from C(═O), and the sulfur atom ring members are independently selected from S, S(O) and S(O)₂, the fused ring optionally substituted with up to 4 substituents independently selected from R¹² on carbon atom ring members and R¹³ on nitrogen atom ring members; provided that when J is J-1, J-2, J-3 or J-6 and a sulfur atom ring member of G¹, G², G³ or G⁶, respectively, is bonded to the ring fusion atom para to the connection of J to the remainder of Formula 1, then said sulfur atom ring member is S (i.e., not S(O) or S(O)₂);
R¹ and R² are independently H, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₄-C₆ alkylcycloalkyl or C₄-C₆ cycloalkylalkyl; or
R¹ and R² are taken together with the carbon to which they are bonded to form a C₃-C₆ saturated carbocyclic ring optionally substituted by C₁-C₃ alkyl, halogen or C₁-C₃ haloalkyl;
R³ is H, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₄-C₆ alkylcycloalkyl or C₄-C₆ cycloalkylalkyl, C₁-C₆ alkoxy, C₁-C₆ haloalkoxy, C₁-C₆ alkylthio, C₁-C₆ haloalkylthio, —SCN, halogen, cyano, nitro, azido, —CO₂H or C₂-C₅ alkoxycarbonyl;
R⁴ is H, C₁-C₆ alkyl, C₁-C₆ haloalkyl or halogen; or
R³ and R⁴ are taken together with the carbon to which they are bonded to form a saturated carbocyclic C₃-C₆ ring optionally substituted by C₁-C₃ alkyl, halogen or C₁-C₃ haloalkyl; or
R¹ and R⁴ are taken together with the carbons to which they are bonded to form a C₃-C₇ saturated carbocyclic ring optionally substituted by C₁-C₃ alkyl, halogen or C₁-C₃ haloalkyl;
R⁵ is H, C₁-C₂ alkyl, halogen, cyano or C₂-C₅ alkoxycarbonyl;
R⁶ is H, C₁-C₂ alkyl or halogen; or
R⁵ and R⁶ are taken together with the carbon to which they are bonded to form a C₃-C₆ saturated carbocyclic ring;
R⁷ is H, —CN, C₂-C₄ alkoxycarbonyl, C₁-C₄ alkylsulfonyl, C₂-C₄ alkylcarbonyl or C₂-C₄ haloalkylcarbonyl;
R^{8a}, R^{8b} and R^{8c} are each independently C₁-C₇ alkyl, C₂-C₇ alkenyl, C₂-C₇ alkynyl, C₃-C₇ cycloalkyl, C₁-C₇ haloalkyl, C₂-C₇ haloalkenyl, C₂-C₇ alkoxyalkyl, C₄-C₇ cycloalkylalkyl, C₃-C₇ haloalkynyl, C₃-C₇ alkylcarbonylalkyl, C₃-C₇ alkoxycarbonylalkyl, C₄-C₇ halocycloalkylalkyl, C₂-C₇ haloalkoxyalkyl, C₂-C₇ alkylthioalkyl, C₂-C₇ alkylsulfonylalkyl, C₂-C₇ alkylsulfinylalkyl, C₂-C₇ cyanoalkyl, C₂-C₇ haloalkylthioalkyl, C₂-C₇ haloalkylsulfonylalkyl, C₂-C₇ haloalkylsulfinylalkyl, C₃-C₇ haloalkoxycarbonylalkyl, C₃-C₇ haloalkylcarbonylalkyl; C₁-C₆ alkylsulfonyl, C₁-C₆ haloalkylsulfonyl or C₃-C₆ cycloalkylsulfonyl; and
R^{9a}, R^{9b}, R^{9c}, R^{9d}, R^{10a}, R^{10b}, R^{11a}, R^{11b} and R^{11c} are each independently H, halogen, cyano, C₁-C₇ alkyl, C₂-C₇ alkenyl, C₂-C₇ alkynyl, C₃-C₇ cycloalkyl, C₄-C₇ cycloalkylalkyl, C₄-C₇ alkylcycloalkyl, C₁-C₇ haloalkyl, C₂-C₇ haloalkenyl, C₃-C₇ haloalkynyl, C₃-C₇ halocycloalkyl, C₁-C₇ alkoxy, C₁-C₇ haloalkoxy, C₂-C₇ alkenyloxy, C₂-C₇ haloalkenyloxy, C₃-C₇ cycloalkoxy, C₃-C₇ halocycloalkoxy, C₂-C₇ alkynyloxy, C₄-C₇ cycloalkylalkoxy, C₄-C₇ halocycloalkylalkoxy, C₁-C₇ alkylthio, C₁-C₇ haloalkylthio, C₁-C₇ haloalkylsulfinyl, C₁-C₇ haloalkylsulfonyl, C₁-C₆ alkylamino, C₂-C₇ dialkylamino, C₁-C₇ haloalkylamino, C₂-C₇ alkylcarbonyl, C₂-C₇ alkoxycarbonyl, C₂-C₇ haloalkylcarbonyl, C₂-C₇ haloalkoxycarbonyl, C₃-C₇ alkylcarbonylalkyl, C₃-C₇, alkoxycarbonylalkyl, aminocarbonyl, C₂-C₈ alkylaminocarbonyl, C₂-C₈ haloalkylaminocarbonyl, C₃-C₈ dialkylaminocarbonyl, C₃-C₈ haloalkyl(alkyl)aminocarbonyl, C₄-C₇ cycloalkylaminocarbonyl, C₅-C₈ cycloalkyl(alkyl)aminocarbonyl, C₄-C₇ halocycloalkylalkyl, C₁-C₇ alkoxyalkyl, C₂-C₇ haloalkoxyalkyl, C₂-C₇ alkylthioalkyl, C₂-C₇ alkylsulfonylalkyl, C₂-C₇ alkylsulfinylalkyl, C₂-C₇ cyanoalkyl, C₂-C₇ haloalkylthioalkyl, C₂-C₇ haloalkylsulfonylalkyl, C₂-C₇ haloalkylsulfinylalkyl, C₃-C₇ haloalkoxycarbonylalkyl, C₃-C₇ haloalkylcarbonylalkyl, C₂-C₇ alkoxyalkoxy, C₂-C₇ haloalkoxyalkoxy, C₂-C₇ alkylthioalkoxy, C₂-C₇ haloalkylthioalkoxy, C₂-C₇ haloalkylsulfonylalkoxy, C₂-C₇ haloalkylsulfinylalkoxy, nitro, C₃-C₁₀ trialkylsilyl, aminosulfonyl, C₁-C₇ alkylaminosulfonyl, C₁-C₇ haloalkylaminosulfonyl, C₂-C₈ dialkylaminosulfonyl, C₃-C₈ haloalkyl(alkyl)aminosulfonyl, C₃-C₆ cycloalkylaminosulfonyl or C₄-C₇ cycloalkyl(alkyl)aminosulfonyl;
each R¹² is independently C₁-C₆ alkyl, C₁-C₆ haloalkyl, C₁-C₆ alkoxy, cyano or halogen;
each R¹³ is independently C₁-C₆ alkyl or C₁-C₆ haloalkyl;
m is 0, 1 or 2; and
n is 0 or 1; provided that the sum of n and m is not more than 2.

More particularly, this invention pertains to a compound of Formula 1 (including all geometric and stereoisomers), an N-oxide or a salt thereof. This invention also relates to a herbicidal composition comprising a compound of Formula 1, or an N-oxide or a salt thereof, wherein the sum of n and m is 1 or 2 (i.e. in a herbicidally effective amount), and (i.e. together with) at least one component selected from the group consisting of surfactants, solid diluents and liquid diluents. This invention further relates to a method for controlling the growth of undesired vegetation comprising contacting the vegetation or its environment with a herbicidally effective amount of a compound of Formula 1, or an N-oxide or a salt thereof, wherein the sum of n and m is 1 or 2 (e.g., as a composition described herein).

### DETAILS OF THE INVENTION

As used herein, the terms “comprises,” “comprising,” “includes,” “including,” “has,” “having”, “contains” “characterized by” or “containing” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim, such a phrase would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally discussed, provided that theses additional materials, steps features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such and invention using the terms “consisting essentially of” or “consisting of”

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

As used herein, the term “alkylating agent” refers to a chemical compound in which a carbon-containing radical is bound through a carbon atom to leaving group such as halide or sulfonate, which is displaceable by bonding of a nucleophile to said carbon atom. Unless otherwise indicated, the term “alkylating” does not limit the carbon-containing radical to alkyl; the carbon-containing radicals in alkylating agents include the variety of carbon-bound substituent radicals specified for R⁵ and R⁶.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy. “Alkenyloxy” includes straight-chain or branched alkenyloxy moieties. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HC≡CCH₂O, CH₃C≡CCH₂O and CH₃C≡CCH₂CH₂O. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Alkylthioalkoxy” denotes alkylthio substitution on alkoxy. “Cyanoalkyl” denotes an alkyl group substituted with one cyano group. Examples of “cyanoalkyl” include NCCH₂, NCCH₂CH₂ and CH₃CH(CN)CH₂. “Alkylamino”, “dialkylamino”, and the like, are defined analogously to the above examples.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkoxy” denotes cycloalkyl linked through an oxygen atom such as cyclopentyloxy and cyclohexyloxy. “Cycloalkylalkoxy” denotes cycloalkylalkyl linked through an oxygen atom attached to the alkyl chain. Examples of “cycloalkylalkoxy” include cyclopropylmethoxy, cyclopentylethoxy, and other cycloalkyl moieties bonded to straight-chain or branched alkoxy groups.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “halocycloalkyl”, “haloalkoxy”, “haloalkylthio”, “haloalkenyl”, “haloalkynyl”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (Cl)₂C═CHCH₂— and CF₃CH₂CH═CHCH₂—. Examples of “haloalkynyl” include HC≡CCHCl—, CF₃C≡C—, CCl₃C≡C— and FCH₂C≡CCH₂—. Examples of “haloalkoxyalkoxy” include CF₃OCH₂O—, ClCH₂CH₂OCH₂CH₂O—, Cl₃CCH₂OCH₂O— as well as branched alkyl derivatives.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or pentoxycarbonyl isomers.

The total number of carbon atoms in a substituent group is indicated by the “Cᵢ-Cⱼ” prefix where i and j are numbers from 1 to 10. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a group contains a substituent which can be hydrogen, for example R¹ or R³, then when this substituent is taken as hydrogen, it is recognized that this is equivalent to said group being unsubstituted. When one or more positions on a group are said to be “not substituted” or “unsubstituted”, then hydrogen atoms are attached to take up any free valency.

When a compound is substituted with a substituent bearing a subscript that indicates the number of said substituents can exceed 1, said substituents (when they exceed 1) are independently selected from the group of defined substituents, e.g., (R¹²)_{q} wherein q is 0, 1, 2 or 3 in Exhibit 1. When a variable group is shown to be optionally attached to a position, for example (R¹²)ₛ wherein s may be 0 in J-1-19 of Exhibit 1, then hydrogen may be at the position even if not recited in the variable group definition.

Unless otherwise indicated, a “ring” or “ring system” as a component of Formula 1 is carbocyclic or heterocyclic. Furthermore, unless otherwise indicated, a “ring” or “ring system” as a component of Formula 1 is saturated or unsaturated. Therefore unless otherwise indicated, the fused rings formed by G¹, G², G³, G⁴, G⁵ and G⁶ are saturated or unsaturated, carbocyclic or heterocyclic rings. The term “ring system” denotes two or more fused rings. The terms “bicyclic ring system” and “fused bicyclic ring system” denote a ring system consisting of two fused rings. The term “fused heterobicyclic ring system” and related terms such as “heterobicyclic” relate to a fused bicyclic ring system in which at least one ring atom is not carbon. The term “ring member” refers to an atom or other moiety (e.g., C(═O), C(═S), S(O) or S(O)₂) forming the backbone of a ring or ring system.

The terms “carbocyclic ring” or “carbocycle” denotes a ring wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring”, “heterocycle” or “heterocyclic ring system” denote a ring or ring system in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen.

Unless described as “fully unsaturated” or “partially unsaturated”, an “unsaturated ring” can be either fully or partially unsaturated. The term “saturated ring” denotes a ring in which no ring member is bonded to an adjacent ring member through a double bond. In regards to degree of saturation, a “partially saturated ring” (alternatively described as a “partially unsaturated ring”) is intermediate between a saturated ring and a fully unsaturated ring (which may be aromatic). Therefore the term “partially saturated ring” denotes a ring comprising at least one ring member bonded to an adjacent ring member through a double bond and also comprising at least one ring member bonded to an adjacent ring member through a single bond that conceptually could be replaced by a double bond to form a less saturated ring. A “fully unsaturated ring” does not comprise a single bond that could be reasonably conceptually replaced by a double bond.

“Aromatic” indicates that each of the ring atoms is essentially in the same plane and has a p-orbital perpendicular to the ring plane, and that (4n+2) it electrons, where n is a positive integer, are associated with the ring to comply with Hückel's rule. Accordingly, neither the dihydro isoxazoline ring of Formula 1 nor the dihydro oxo azinyl rings (the rings directly bonded to remainder of Formula 1) in the heterobicyclic ring systems of J-1, J-2, J-3, J-4, J-5 and J-6 are aromatic.

As used herein, the following definitions shall apply unless otherwise indicated. The term “optionally substituted” is used interchangeably with the phrase “substituted or unsubstituted”. Unless a limitation is indicated, an optionally substituted group (e.g., the ring formed by R¹ and R² taken together with the carbon to which they are bonded) can have a substituent at each substitutable position of the group, and each substitution is independent of the other. If a limitation of number of optional substituents is indicated (e.g., up to 4 substituents on the fused ring formed by G¹, G², G³, G⁴, G⁵ or G⁶), then the number of substituents can vary from zero up to the limit.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of undesired vegetation (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Compounds of Formula 1 and N-oxides and salts thereof typically exist in more than one form, and Formula 1 and N-oxides and salts thereof thus include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 or an N-oxide or salt thereof can exhibit beneficial effects (e.g., suitability for preparation of useful formulations, improved biological performance) relative to another polymorph or a mixture of polymorphs of the same compound of Formula 1 or the N-oxide or salt thereof. Preparation and isolation of a particular polymorph of a compound of Formula 1 or an N-oxide or salt thereof can be achieved by methods known to those skilled in the art including, for example, crystallization using selected solvents and temperatures.

Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.
Embodiment 1. A compound of Formula 1 wherein R¹ is methyl or chloromethyl (i.e. mono chloromethyl).
Embodiment 2. A compound of Embodiment 1 wherein R¹ is methyl.
Embodiment 3. A compound of Embodiment 1 wherein R¹ is chloromethyl.
Embodiment 4. A compound of Formula 1 or any one of Embodiments 1 through 3 wherein R² is methyl.
Embodiment 5. A compound of Formula 1 or any one of Embodiments 1 through 4 wherein R³ is H.
Embodiment 6. A compound of Formula 1 or any one of Embodiments 1 through 5 wherein R⁴ is H.
Embodiment 7. A compound of Formula 1 or any one of Embodiments 1 through 6 wherein R⁵ is H.
Embodiment 8. A compound of Formula 1 or any one of Embodiments 1 through 7 wherein R⁶ is H.
Embodiment 9. A compound of Formula 1 or any one of Embodiments 1 through 8 wherein the sum of n+m is greater than 0.
Embodiment 10. A compound of Formula 1 or any one of Embodiments 1 through 9 wherein n is 0.
Embodiment 11. A compound of Embodiment 10 wherein m is 2.
Embodiment 12. A compound of Embodiment 10 wherein m is 1.
Embodiment 13. A compound of Formula 1 or any one of Embodiments 1 through 8 wherein n and m are both 0.
Embodiment 14. A compound of Formula 1 or any one of Embodiments 1 through 13 wherein independently each G¹, G² and G⁵ taken together with the two ring fusion atoms to which each is bonded forms a fused pyrroline, pyrrolidine, thiazoline, thiazolidine, imidazoline, imidazolidine, pyrazoline, pyrazolidine, triazoline, triazolidine, dihydropyridine, dihydropyrimidine, dihydropyrazine, dihydropyridazine, oxazoline, oxazolidine, oxazine, thiazine, isoxazoline, isoxazolidine, isothiazoline, isothiazolidine, piperidine, piperazine or diazepine ring, and independently each G³, G⁴ and G⁶ taken together with the two ring fusion atoms to which each is bonded forms a fused benzene, cyclohexane, cyclohexene, cyclopentane, cyclopentene, thiophene, dihydrothiophene, furan, dihydrofuran, pyrrole, pyrazoline, pyrrolidine, thiazole, thiazoline, thiazolidine, imidazole, imidazoline, imidazolidine, pyrazole, pyrazoline, pyrazolidine, triazole, triazoline, triazolidine, cycloheptane, pyridine, pyrimidine, pyrazine, pyridazine, dioxane, oxazine, thiazine, pyran, oxepin, oxazole, oxazoline, isoxazole, isoxazoline, isothiazole, isothiazoline, piperidine, piperazine or azepine ring; each ring optionally substituted with up to 4 substituents independently selected from R¹² on carbon atom ring members and R¹³ on nitrogen atom ring members.
Embodiment 15. A compound of Formula 1 or any one of Embodiments 1 through 14 wherein independently the fused ring formed by G¹, G², G³, G⁴, G⁵ or G⁶ is optionally substituted with up to 3 substituents selected from selected from R¹² on carbon ring atom members and R¹³ on nitrogen atom ring members.
Embodiment 16. A compound of Embodiment 15 wherein independently the fused ring formed by G¹, G², G³, G⁴, G⁵ or G⁶ is optionally substituted with up to 2 substituents selected from selected from R¹² on carbon ring atom members and R¹³ on nitrogen atom ring members.
Embodiment 17. A compound of Formula 1 or any one of Embodiments 1 through 16 wherein J is J-1, J-2, J-3, J-4 or J-5.
Embodiment 18. A compound of Formula 1 or any one of Embodiments 1 through 16 wherein J is J-1, J-2, J-3, J-4 or J-6.
Embodiment 19. A compound of Formula 1 or any one of Embodiments 1 through 16 wherein J is J-1, J-2, J-4, J-5 or J-6.
Embodiment 20. A compound of Formula 1 or any one of Embodiments 1 through 16 wherein J is J-1, J-3, J-4, J-5 or J-6.
Embodiment 21. A compound of Formula 1 or any one of Embodiments 1 through 16 wherein J is J-1, J-3, J-4, J-5 or J-6.
Embodiment 22. A compound of Formula 1 or any one of Embodiments 1 through 16 wherein J is J-2, J-3, J-4, J-5 or J-6.
Embodiment 23. A compound of Formula 1 or any one of Embodiments 1 through 16 wherein J is J-1, J-2, J-3 or J-6.
Embodiment 24. A compound of Embodiment 23 wherein J is J-1, J-2 or J-3.

Embodiment 25. A compound of Embodiment 24 wherein J is J-1 or J-2.
Embodiment 26. A compound of Embodiment 24 wherein J is J-1 or J-3.
Embodiment 27. A compound of Embodiment 23 wherein J is J-1 or J-6.
Embodiment 28. A compound of Formula 1 or any one of Embodiments 1 through 16 wherein J is J-1.
Embodiment 29. A compound of Formula 1 or any one of Embodiments 1 through 16 wherein J is J-2
Embodiment 30. A compound of Formula 1 or any one of Embodiments 1 through 16 wherein J is J-3.
Embodiment 31. A compound of Formula 1 or any one of Embodiments 1 through 16 wherein J is J-4.
Embodiment 32. A compound of Formula 1 or any one of Embodiments 1 through 16 wherein J is J-5.
Embodiment 33. A compound of Formula 1 or any one of Embodiments 1 through 16 wherein J in J-6.
Embodiment 34. A compound of Formula 1 or any one of Embodiments 1 through 13 wherein J is selected from J-1-1 through J-1-22, J-2-1 through J-2-15, J-3-1 through J-3-20, J-4-1 through J-4-20, and J-5-1 through J-5-15 depicted in Exhibit 1

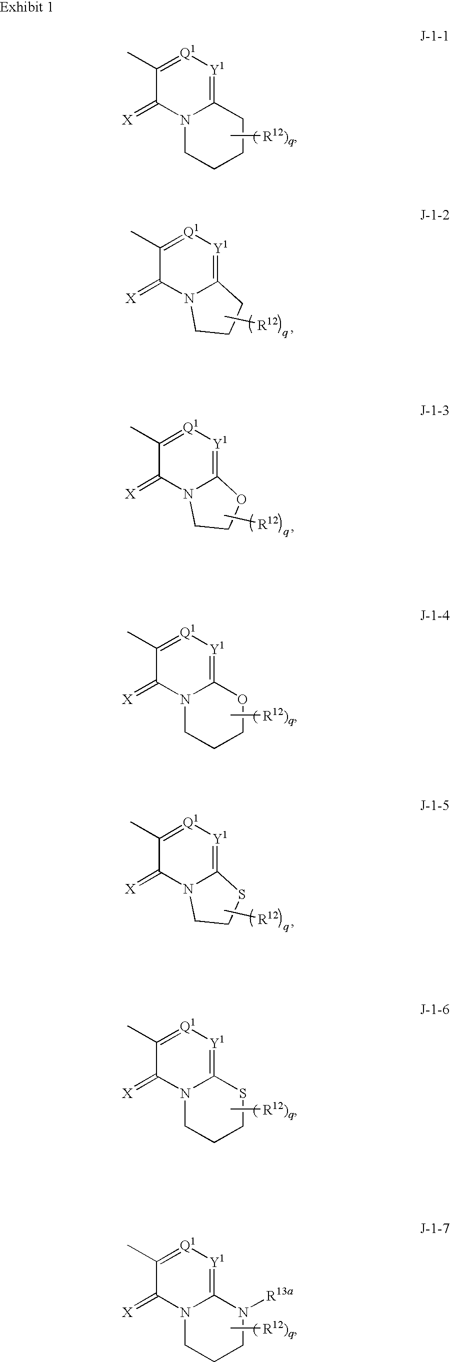

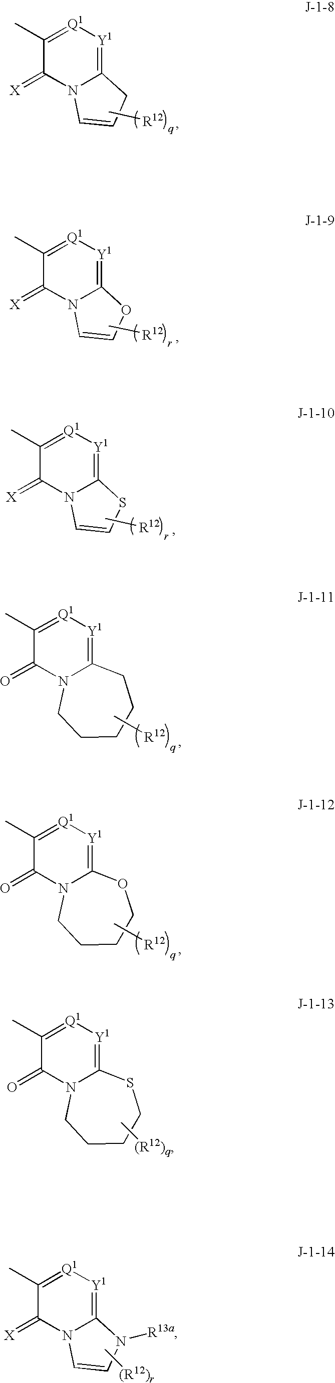

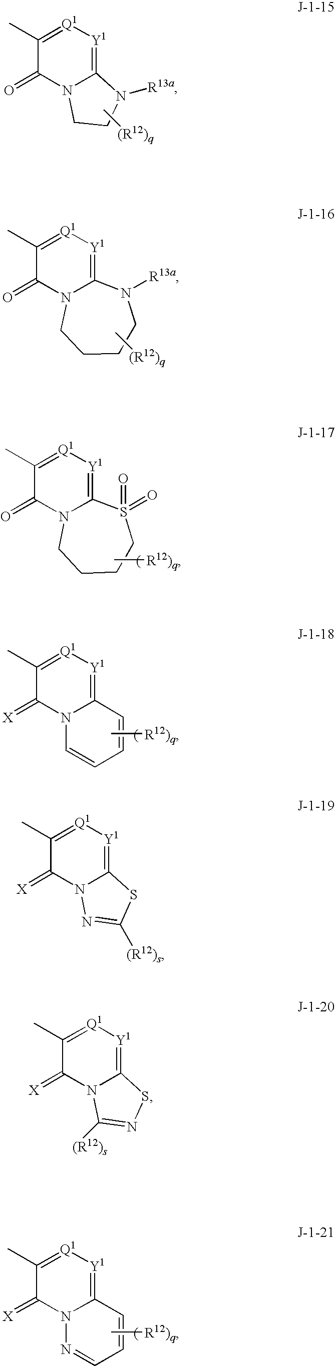

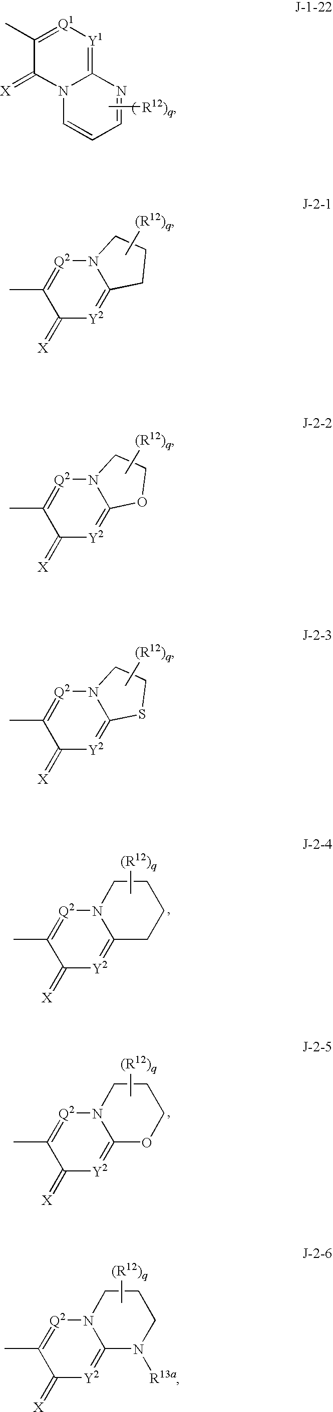

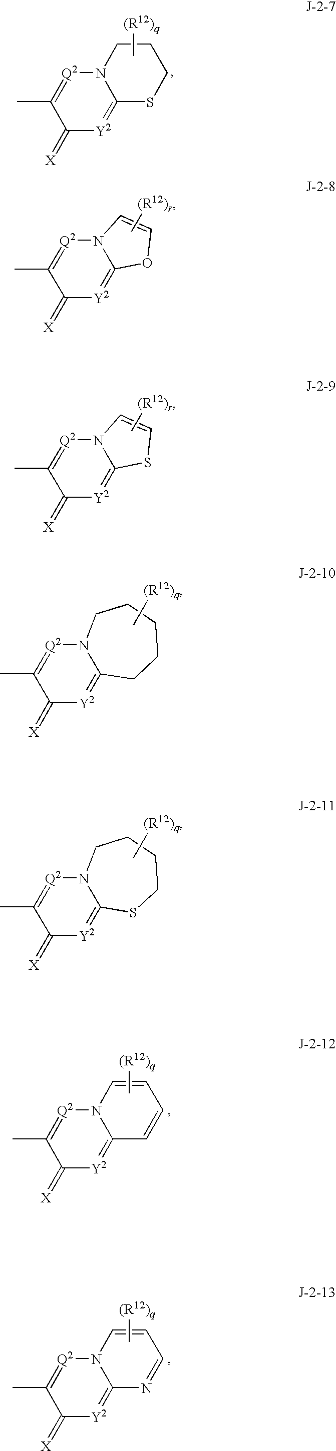

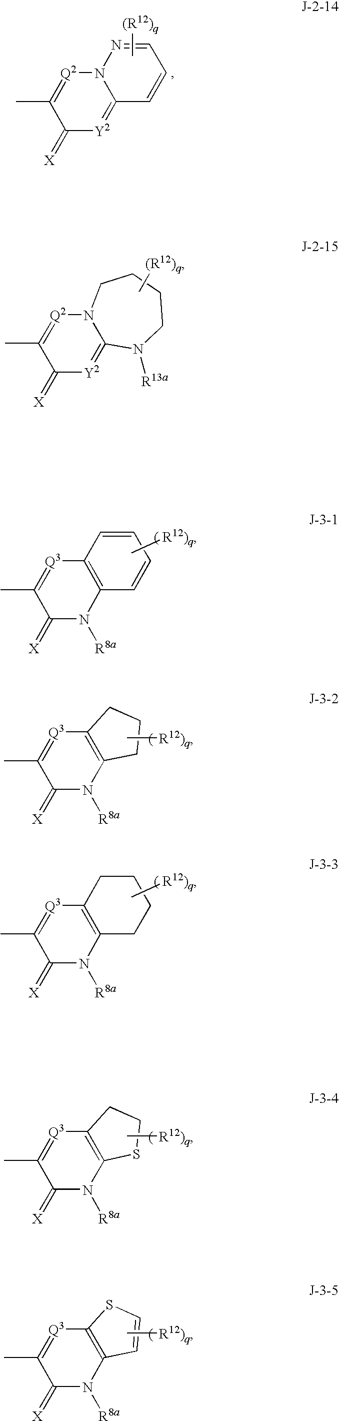

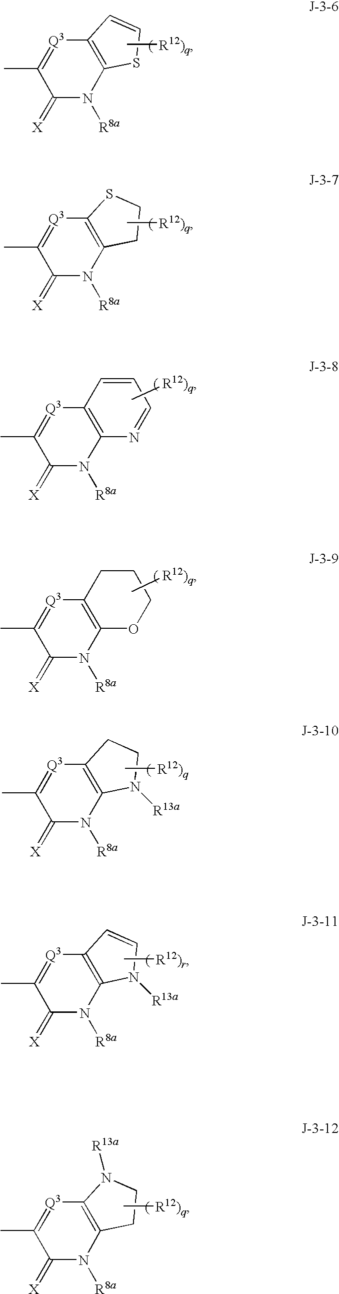

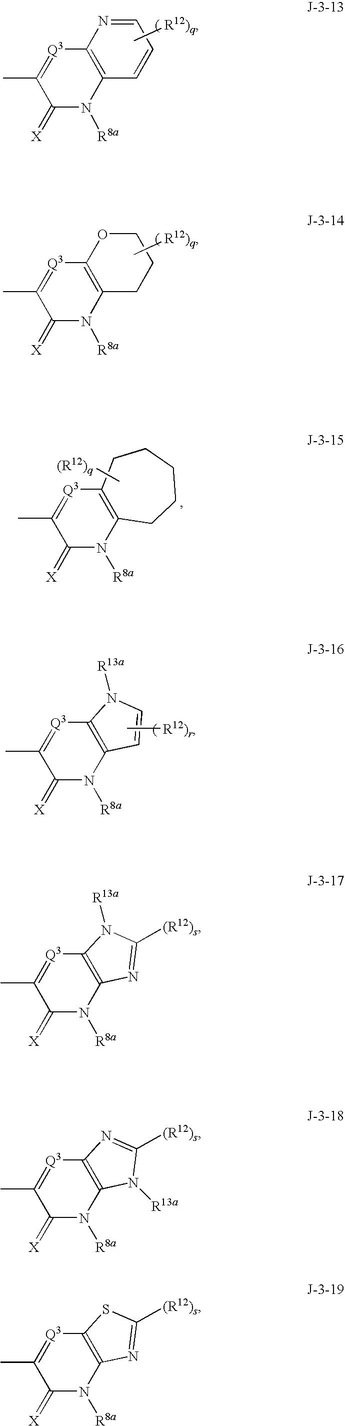

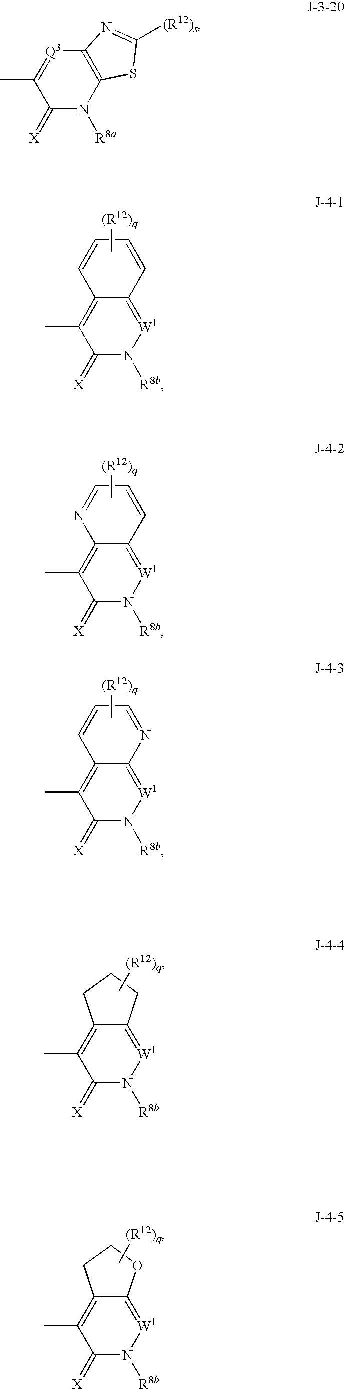

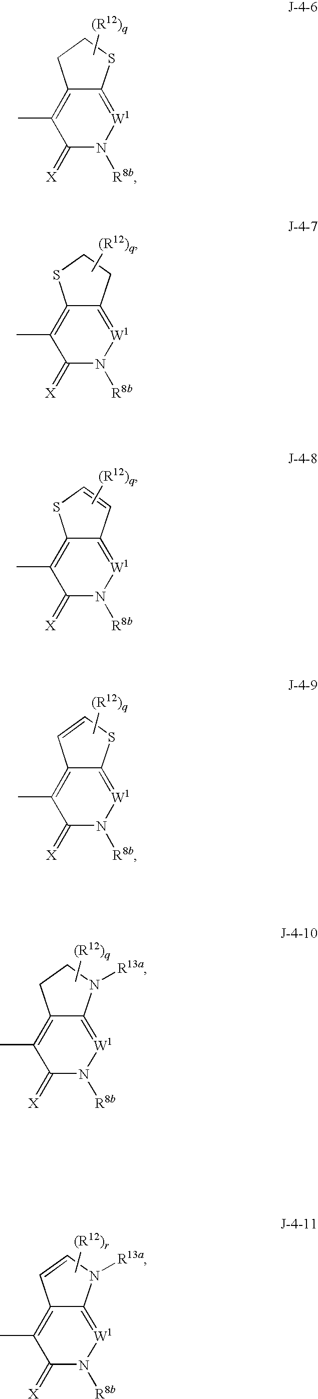

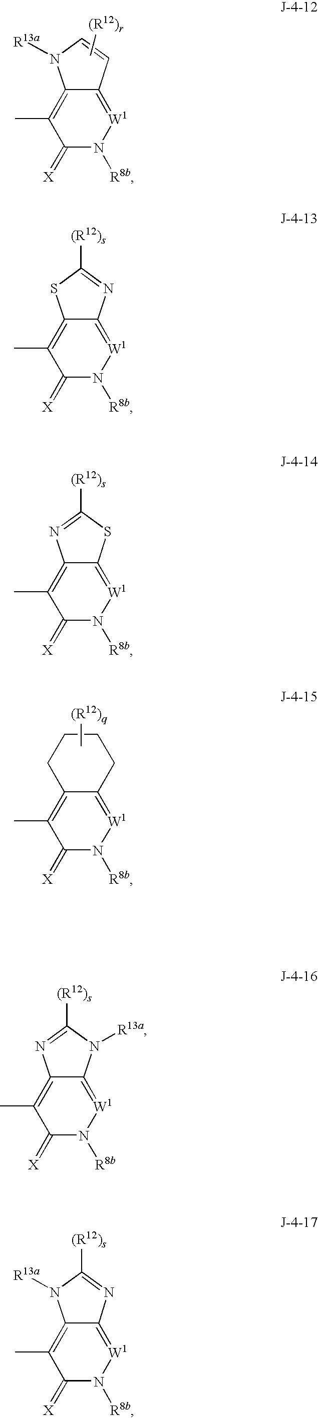

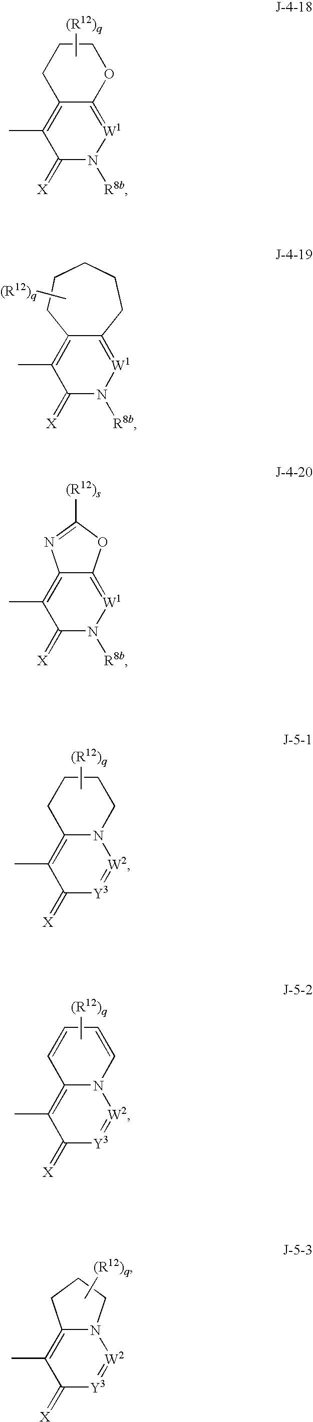

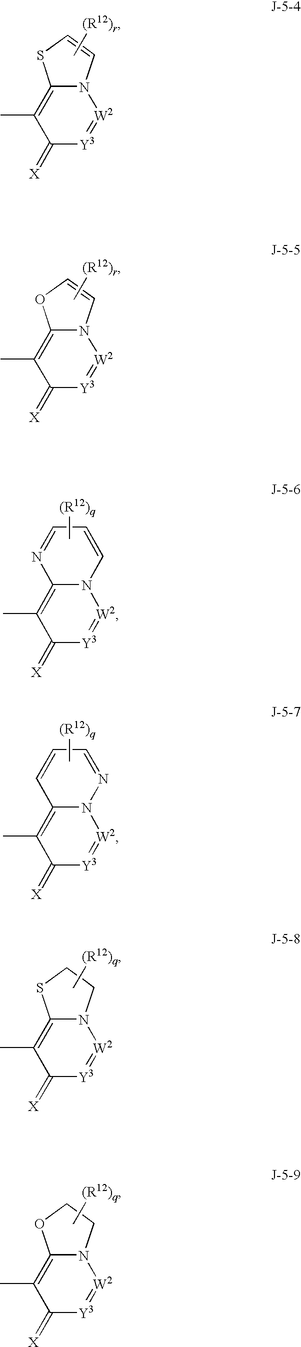

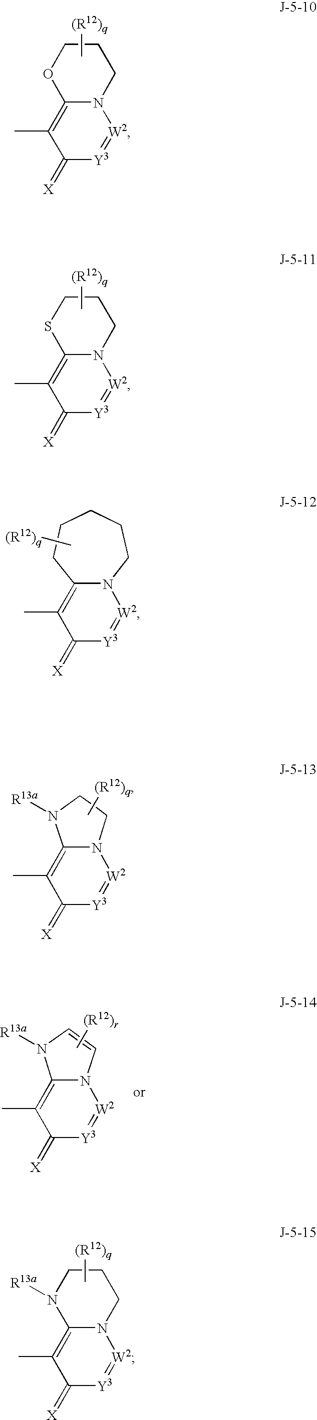

R^{13a} is H or R¹³;
q is 0, 1, 2 or 3;
r is 0, 1 or 2; and
s is 0 or 1.
Embodiment 35. A compound of Embodiment 34 wherein J is selected from J-1-1 through J-1-22.
Embodiment 36. A compound of Embodiment 34 wherein J is selected from J-2-1 through J-2-15.
Embodiment 37. A compound of Embodiment 34 wherein J is selected from J-3-1 through J-3-20.
Embodiment 38. A compound of Embodiment 34 wherein J is selected from J-4-1 through J-4-20.
Embodiment 39. A compound of Embodiment 34 wherein J is selected from J-5-1 through J-5-15.
Embodiment 40. A compound of any one of Embodiments 34 through 39 wherein q is 0, 1 or 2.
Embodiment 41. A compound of Formula 1 or any one of Embodiments 1 through 40 wherein X is O.
Embodiment 42. A compound of Formula 1 or any one of Embodiments 1 through 41 wherein independently R^{8a}, R^{8b} and R^{8c} are C₁-C₂ alkyl.
Embodiment 43. A compound of Formula 1 or any one of Embodiments 1 through 42 wherein independently R^{9a}, R^{9b}, R^{9c} and R^{9d} are CF₃, OCHF₂, OCH₂CF₃, F, Br or Cl.
Embodiment 44. A compound of Embodiment 43 wherein independently R^{9a}, R^{9b}, R^{9c} and R^{9d} are CF₃, OCHF₂, OCH₂CF₃ or Cl.
Embodiment 45. A compound of Formula 1 or any one of Embodiments 1 through 44 wherein independently R^{10a}, R^{10b}, R^{11a}, R^{11b} and R^{11c} are H, CH₃, CH₂CH₃, cyclopropyl, OCH₃ or OCH₂CH₃.
Embodiment 46. A compound of Formula 1 or any one of Embodiments 1 through 45 wherein independently R^{11a}, R^{11b} and R^{11c} are H.
Embodiment 47. A compound of Formula 1 or any one of Embodiments 1 through 46 wherein independently Q¹ is CR^{9a}, Q² is CR^{9b}, Q³ is CR^{9c} and Q⁴ is CR^{9d}.
Embodiment 48. A compound of Formula 1 or any one of Embodiments 1 through 47 wherein when J is J-1, then Q¹ is CR^{9a} and Y¹ is N, or Q¹ is CR^{9a} and Y¹ is CR^{11a}.
Embodiment 49. A compound of Embodiment 48 wherein when J is J-1, then Q¹ is CR^{9a} and Y¹ is N.
Embodiment 50. A compound of Embodiment 48 wherein when J is J-1, then Q¹ is CR^{9a} and Y¹ is CR^{11a}.
Embodiment 51. A compound of Formula 1 or any one of Embodiments 1 through 50 wherein when J is J-2, then Q² is CR^{9c} and Y² is N, or Q³ is CR^{9b} and Y² is CR^{11b}.
Embodiment 52. A compound of Embodiment 51 wherein when J is J-2, then Q² is CR^{9b} and Y² is N.
Embodiment 53. A compound of Embodiment 51 wherein when J is J-2, then Q² is CR^{9b} and Y² is CR^{11b}.
Embodiment 54. A compound of Formula 1 or any one of Embodiments 1 through 53 wherein when J is J-3, then Q³ is CR^{9c}.
Embodiment 55. A compound of Formula 1 or any one of Embodiments 1 through 54 wherein when J is J-4, then W¹ is CR^{10a} or N.
Embodiment 56. A compound of Embodiment 55 wherein when J is J-4, then W¹ is CR^{10a}.
Embodiment 57. A compound of Embodiment 55 wherein when J is J-4, then W¹ is N.
Embodiment 58. A compound of Formula 1 or any one of Embodiments 1 through 57 wherein when J is J-5, then W² is CR^{10b} and Y³ is N, or W² is N and Y³ is CR^{11c}, or W² is R^{10b} and Y³ is CR^{11c}.
Embodiment 59. A compound of Embodiment 58 wherein when J is J-5, then W² is CR^{10b} and Y³ is N, or W² is N and Y³ is CR^{11c}.
Embodiment 60. A compound of Embodiment 59 wherein when J is J-5, then W² is CR^{10b} and Y³ is N.
Embodiment 61. A compound of Embodiment 59 wherein when J is J-5, then W² is N and Y³ is CR^{11c}.
Embodiment 62. A compound of Formula 1 or any one of Embodiments 1 through 61 wherein when J is J-6, then Q⁴ is CR^{9d}.
Embodiment 63. A compound of Formula 1 or any one of Embodiments 1 through 13 wherein J is selected from Table 1 (below).

Embodiments of this invention, including Embodiments 1-63 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-63 above as well as any other embodiments described herein, and any combination thereof, pertain to the compositions and methods of the present invention.

Combinations of Embodiments 1-63 are illustrated by:
Embodiment A. A compound of Formula 1 wherein R¹ is methyl, R² is methyl, R³ is H and R⁴ is H.
Embodiment B. A compound of Formula 1 wherein R¹ is methyl, R² is chloromethyl, R³ is H and R⁴ is H.

Specific embodiments include compounds of Formula 1 selected from the group consisting of:
3-[[(4,5-dihydro-5,5-dimethyl-3-isoxazolyl)sulfonyl]methyl]-7,8-dihydro-2-(trifluoromethyl)pyrrolo[1,2-α]pyrimidin-4(6H)-one;
3-[[(4,5-dihydro-5,5-dimethyl-3-isoxazolyl)sulfonyl]methyl]-6,7,8,9-tetrahydro-2-(trifluoromethyl)-4H-pyrido[1,2-α]pyrimidin-4-one; and
6-[[(4,5-dihydro-5,5-dimethyl-3-isoxazolyl)sulfonyl]methyl]-2,3-dihydro-7-(trifluoromethyl)-5H-oxazolo[3,2-α]pyrimidin-5-one.

Of note is a compound of Formula 1 or any one of the preceding embodiments wherein Formula 1 does not include N-oxides thereof. Also of note is a compound of Formula 1 or any one of the preceding embodiments wherein Formula 1 does not include N-oxides or salts thereof.

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above. Also of note is the use of compounds of the invention for selective control of weeds in wheat, barley, and particularly maize, rice (both upland and paddy), soybeans and wheat.

Also noteworthy as embodiments are herbicidal compositions of the present invention comprising the compounds of embodiments described above.

One or more of the following methods and variations as described in Schemes 1-21 can be used to prepare the compounds of Formula 1. The definitions of J, R¹, R², R³, R⁴, R⁵, R⁶, R^{8a}, R^{8b}, R^{8c}, R^{9a}, R^{9b}, R^{9c}, R^{9d}, R^{10a}, R^{10b}, R^{11a}, R^{11b}, R^{11c}, G¹, G², G³, G⁴, G⁵, G⁶, X, m and n in the compounds of Formulae 1-26 below are as defined above in the Summary of the Invention unless otherwise noted. Formulae 1a-1e are various subsets of Formula 1, and all substituents for Formulae 1a-1e are as defined above for Formula 1 unless otherwise noted. Formulae 6a, 6b, 6c, 6d, 6e and 6f are various subsets of Formula 6, and all substituents for Formulae 6a-6f are as defined for Formula 6 unless otherwise noted.

Sulfoxides and sulfones of Formula 1 where m is 1 or 2 and n is 0 can be made via oxidation of the linking sulfur atom on sulfides of Formula 1a (i.e. Formula 1 where m and n are both 0). As illustrated in Scheme 1, compounds of Formula 1b (i.e. Formula 1 wherein n is 0) wherein m is 1 (i.e. sulfoxides) or 2 (i.e. sulfones) are prepared by oxidizing sulfides of Formula 1a with a suitable oxidizing agent. In a typical procedure, an oxidizing agent in an amount from 1 to 4 equivalents depending on the oxidation state of the product desired is added to a solution of the compound of Formula 1a in a solvent. Useful oxidizing agents include Oxone® (potassium peroxymonosulfate), hydrogen peroxide, sodium periodate, peracetic acid and 3-chloroperbenzoic acid. The solvent is selected with regard to the oxidizing agent employed. Aqueous ethanol or aqueous acetone is preferably used with potassium peroxymonosulfate, and dichloromethane is generally preferable with 3-chloroperbenzoic acid. Useful reaction temperatures typically range from 0 to 90° C. Particular procedures useful for oxidizing sulfides to sulfoxides and sulfones are described by Brand et al., J. Agric. Food Chem. 1984, 32, 221-226 and references cited therein.

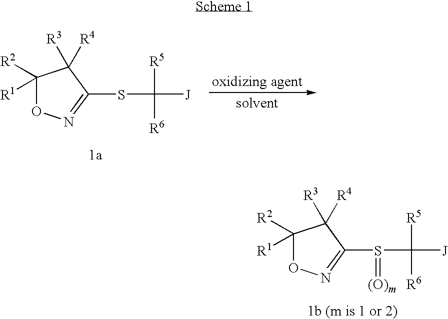

As shown in Scheme 2, sulfoximines of Formula 1c (i.e. Formula 1 wherein m and n are 1 and R⁷ is H) can be prepared from corresponding sulfoxides of Formula 1b (i.e. Formula 1 where m is 1 and n is 0) by treatment with hydrazoic acid. The hydrazoic acid is conveniently generated in situ from sodium azide and sulfuric acid. In a typical procedure, sodium azide is added to a mixture of a sulfoxide, concentrated sulfuric acid and a suitable solvent for the sulfoxide such as dichloromethane or chloroform. Useful temperatures range from room temperature to the reflux temperature of the solvent.

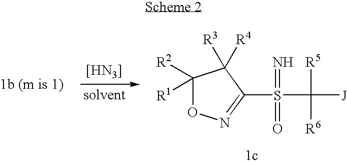

As shown in Scheme 3, substituted sulfoximines of Formula 1d (i.e. Formula 1 where m and n are 1 and R⁷ is other than H) can be prepared from corresponding sulfoximines of Formula 1c by reaction with an appropriate electrophilic reactant comprising R⁷. In the present context, “electrophilic reactant comprising R⁷” means a reactant capable of transferring R⁷ to form a bond with a nucleophile (in this case the sulfoximine nitrogen). Many electrophilic reactants comprising R⁷ correspond to the formula R⁷X¹ wherein X¹ is a nucleophilic reaction leaving group, also known as a nucleofuge. Common nucleofuges, i.e. X¹, include, for example, halides such as Cl, Br and I, and sulfonates such as methanesulfonate, trifluoromethanesulfonate and 4-methylbenzenesulfonate. Reactions with electrophilic reactants of the formula R⁷X¹ are often conducted in the presence of a base as well as a solvent. For example, reaction of a compound of Formula 1c with cyanogen bromide (BrCN) in the presence of base gives the compound of Formula 1d where R⁷ is cyano. As further examples, reaction of a compound of Formula 1c with an alkylcarbonyl halide, an alkoxycarbonyl halide or an alkylsulfonyl halide in the presence of a base gives the corresponding compound of Formula 1d where R⁷ is alkylcarbonyl, alkoxycarbonyl or alkylsulfonyl, respectively.

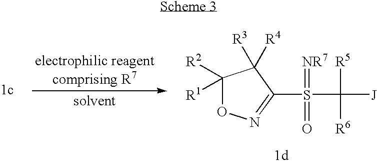

As shown in Scheme 4, sulfilimines of Formula 1e (i.e. Formula 1 where m is 0, n is 1 and R⁷ is cyano or a radical bonded through a carbonyl or sulfonyl moiety) can be made by reaction of a sulfide of Formula 1a with a compound of formula R⁷NH₂ in the presence of a suitable oxidizing agent such as iodobenzene diacetate in a solvent such as dichloromethane.

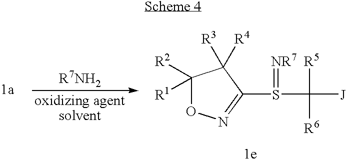

A variety of general procedures have been reported in the literature for converting sulfoxides to sulfoximines, and sulfides to sulfilimines; see, for example, U.S. Patent Publication 2005/0228027, PCT Patent Publication WO 2006/037945, Organic Letters 2004, 6(8) 1307-1307, Organic Letters 2006, 8(11), 2349-2352, and Synlett 2002, (1), 116-118.

An alternative method for preparing compounds of Formula 1 wherein at least one of R⁵ and R⁶ is other than hydrogen is illustrated in Scheme 5. This method involves deprotonating corresponding compounds of Formula 1 wherein at least one of R⁵ and R⁶ is hydrogen and the sum of m and n is greater than 0 with a base to form a carbanion, followed by addition of electrophilic reactant(s) to the carbanion to provide the desired R⁵ and R⁶ substituents. This method is particularly useful for preparing compounds of Formula 1 wherein R⁵ or R⁶ is halogen or alkyl. In the method of Scheme 5, the starting compound of Formula 1 (wherein at least one of R⁵ and R⁶ is hydrogen and the sum of m and n is greater than 0) is reacted with a suitable base such as sodium hydroxide, sodium hydride, potassium t-butoxide or n-butyllithium in an appropriate solvent include, for example, tetrahydrofuran, diethyl ether, dioxane, dichloromethane or N,N-dimethylformamide. One skilled in the art knows the appropriate electrophilic reactants (i.e. alkylating agents) to provide particular R⁵ and R⁶ substituents. The amount of base and alkylating agents required in this reaction (generally 1 to 4 equivalents of base and/or alkylating agent) will depend on whether the starting compound of Formula 1 in Scheme 5 is to be monoalkylated (to obtain a compound of Formula 1 where at least one R⁵ or R⁶ is other than H) or dialkylated (to obtain a compound of Formula 1 where both R⁵ or R⁶ are other than H). The reaction is typically run at temperatures ranging from −78° C. to the reflux temperature of the solvent, depending upon the base and solvent used. Examples of reactions analogous to those shown in Scheme 5 are described by A. Volonterio et al., Tetrahedron Letters 2005, 46(50), 8723-8726 and S. Ostrowski et al., Heterocycles 2005, 65(10), 2339-2346.

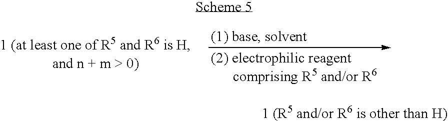

As shown in Scheme 6, sulfides of Formula 1a can be made by the reaction of a isoxazoline isothiourea salt of Formula 2 (where X² is a chloride or bromide counterion) with a heterocyclic alkyl electrophile of Formula 3 wherein X³ is leaving group such as a halogen or a sulfonate (e.g., methanesulfonate) in the presence of excess base in a suitable solvent. In a typical procedure, the isothiourea salt of Formula 2 (which can be regarded as a protected form of a thiol nucleophile) and an electrophilic agent of Formula 3 are combined in a suitable solvent such as acetonitrile, ethanol, tetrahydrofuran, dioxane, dichloromethane, N,N-dimethylformamide or toluene followed by the addition of base such as potassium carbonate, sodium hydride, sodium or potassium hydroxide, pyridine, lithium diisopropylamide or triethylamine. The reaction can be run under a wide range of temperatures, with optimum temperatures typically ranging from 0° C. to the reflux temperature of the solvent. Generally, at least two equivalents of base are used for neutralizing the two equivalents of acid (i.e. HX² and HX³) that is generated in this reaction.

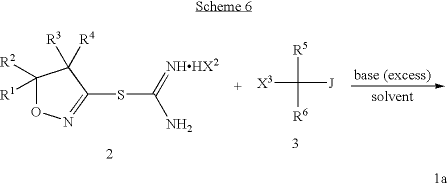

Isoxazoline isothiourea salts of Formula 2 can be prepared by the method reported in U.S. Patent Publication US 2007/0185334 A1 and European Patent Publication EP 1 829 868 A1. One skilled in the art will recognize that the free 3-thiol form of 2 or other protected forms of the thiol (e.g., 3-acetylthioisoxazoline derivative) can also be used in place of 2 in the reaction shown in Scheme 6.

Alternatively, sulfides of Formula 1a can be prepared by the method illustrated in Scheme 7, in which isoxazolines of Formula 4 wherein X⁴ is a suitable leaving group, such as halogen or methanesulfonate are allowed to react with a heterocyclic alkylisothiourea salt of Formula 5. In a typical procedure a compound of Formula 4 is mixed with a compound of Formula 5 in the presence of excess base (generally 2 to 4 equivalents) such as potassium carbonate, sodium hydride, lithium diisopropylamide, pyridine or triethylamine in a variety of solvents including acetonitrile, tetrahydrofuran, diethyl ether, dichloromethane, dioxane, N,N-dimethylformamide and toluene. Optimum reaction temperatures typically range from 0° C. to the reflux temperature of the solvent.

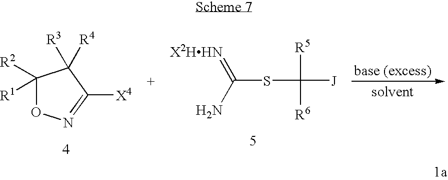

Isoxazolines of Formula 4 where X⁴ is halogen can be made by the method reported in U.S. Patent Publication 2007/0185334 and PCT Patent Publication WO 2007/0965776, and isoxazolines of Formula 4 where X⁴ is methylsulfonyl can be made by the method reported in European Patent Publication EP 1203768 and PCT Patent Publication WO 2003/010165. One skilled in the art will also recognize that the free 3-thiol form of 5 or other protected forms of the thiol (e.g., 3-acetylthioisoxazoline derivative) can also be used in place of 5 in the reaction shown in Scheme 7.

Sulfides of Formula 1a can also be prepared by the one pot, two-step method shown in Scheme 8, in which an isoxazoline of Formula 4 is treated with a thiolating agent such thiourea or sodium hydrosulfide, and the generated intermediate is reacted in situ with a compound of Formula 3 in the presence of a base. In a typical procedure an isoxazoline of Formula 4 is combined with a thiolating agent in a solvent such as ethanol, tetrahydrofuran, dioxane, dichloromethane, N,N-dimethylformamide or toluene, followed by addition of a suitable base, such as sodium hydride, sodium or potassium hydroxide, pyridine, lithium diisopropylamide, triethylamine or potassium carbonate, and a compound of Formula 3. The reaction can be run under a wide range of temperatures with optimum temperatures ranging from 0° C. to the reflux temperature of the solvent. Examples of reactions analogous to the method of Scheme 8 are taught in U.S. Patent Publication 2004/0110749 A1 and PCT Patent Publications WO 2006/123088 and WO 2007/003295.

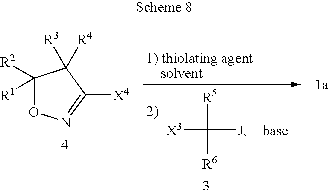

One skilled in the art will recognize that the order of addition of compounds of Formulae 3 and 4 in the reaction shown in Scheme 8 can be reversed so that the compound of Formula 3 is thiolated in a suitable solvent prior to addition of the compound of Formula 4 and base.

As shown in Scheme 9, intermediates of Formula 3 where X³ is halogen can be made by “benzylic” halogenation of precursors of Formula 6 with an N-halosuccinimide in an appropriate solvent (e.g., N,N-dimethylformamide, carbon tetrachloride, acetonitrile or dichloromethane), generally in the presence of a radical-generating catalyst such as benzoyl peroxide or 2,2′-azobis(2-methylpropionitrile) (AIBN). Compounds of Formula 3 are also obtained by contacting alcohols of Formula 7 with a halogen-containing reagent such as phosphorus(V) oxychloride or phosphorus tribromide in the presence of triphenylphosphine in an appropriate solvent (e.g., toluene, carbon tetrachloride, dichloroethane or dichloromethane). Alcohols of Formula 7 can also be reacted with an appropriate sulfonylating reagent in the presence of base and solvent (e.g., pyridine, triethylamine or potassium carbonate in tetrahydrofuran, dioxane, dichloromethane, N,N-dimethylformamide or toluene) to give compounds of Formula 3 where X³ is a sulfonate leaving group such as methanesulfonate.

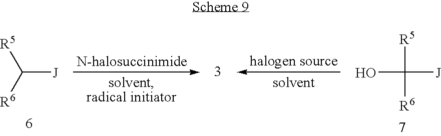

Precursors to alcohols of Formula 7 where R⁵ and R⁶ are H are generally aldehydes of Formula 8 or alkyl esters of Formula 9. Reduction of compounds of Formulae 8 or 9 with an appropriately selected reducing agent (e.g., lithium borohydride, sodium borohydride or diisobutylaluminum hydride) in a compatible solvent (e.g., tetrahydrofuran, methanol, diethyl ether) provides compounds of Formula 7 as shown in Scheme 10. Esters of Formula 9 can also be reacted with Grignard reagents to give compounds of Formula 7 where R⁵ and R⁶ are other than H.

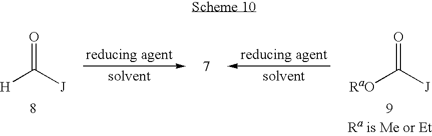

As shown in Scheme 11, intermediates of Formula 6a (Formula 6 where J is J-1, X is O, Q¹ is CR^{9a}, Y¹ is N) and Formula 6b (Formula 6 where J is J-2, X is O, Q² is CR^{9b}, Y² is N) can be made by cyclization of cyclic amidines of Formula 10 with ketoesters of Formula 11. Cyclization of a cyclic amidine of Formula 10, wherein the fused ring formed by G is saturated, with a ketoester of Formula 11 is preferably carried out in the presence of a base such as sodium bicarbonate, potassium carbonate, sodium acetate, sodium hydride or sodium methoxide and an appropriate solvent (e.g., xylenes, toluene, ethanol, tetrahydrofuran, dioxane, dichloromethane, N,N-dimethylformamide or methanol) at temperatures ranging from 0° C. to the reflux temperature of the solvent. Condensation of a cyclic amidine of Formula 10, wherein the fused ring formed by G is unsaturated, with a ketoester of Formula 11 is preferably conducted under acidic conditions in the presence of polyphosphoric acid or sulfuric acid. The acid-catalyzed reaction can be carried out neat or in a solvent such as xylene or toluene. This condensation can form mixtures of the compounds of Formulae 6a and 6b or exclusively one product, depending on the reactants and reaction conditions used.

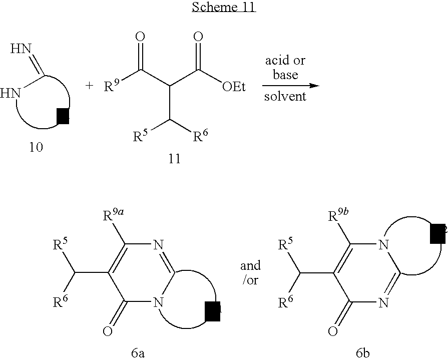

wherein G in Formula 10 corresponds to G¹ in Formula 6a and G² in Formula 6b, and R⁹ in Formula 11 corresponds to R^{9a} in Formula 6a and R^{9b} in Formula 6b.

Bicyclic intermediates of Formula 6a wherein the ring member of G¹ bonded to the ring fusion atom para to the connection of the pyrimidinone ring to —CHR⁵R⁶ is an oxygen, sulfur or nitrogen atom ring member can also be made by the method shown in Scheme 12. (In this Scheme, the para-bonded oxygen, sulfur or nitrogen atom member corresponds to Q in Formula 13.) Reacting a ketoester of Formula 11 with a urea, thiourea or guanidine of Formula 12 wherein Q is O, S or NH in the presence of a suitable base and solvent, such as those described for the method of Scheme 11, gives a pyrimidinone of Formula 13 that can be alkylated with a bis-electrophile of Formula 14 wherein the two X⁵ leaving groups are independently halogen or mesylate. This bis-alkylation is preferably conducted in a solvent such as ethanol, tetrahydrofuran, dioxane, dichloromethane, N,N-dimethylformamide, methanol or toluene in the presence of a suitable base, such as sodium hydride, sodium or potassium hydroxide, pyridine, lithium diisopropylamide, triethylamine or potassium carbonate. The reaction can be run at temperatures ranging from 0° C. to the reflux temperature of the solvent.

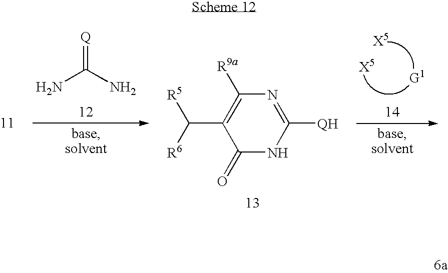

Q is O, S or NH

Alternatively, intermediates of Formula 6a can be made by the reaction illustrated in Scheme 13 involving coupling an imidate of Formula 15, wherein X⁶ is nucleofuge such as halogen, with a ketoester enamine of Formula 16 in the presence of a suitable base and solvent such as those described for the method of Scheme 11. Ketoester enamines of Formula 16 are readily prepared by reacting the corresponding ketoesters of Formula 11 with ammonia.

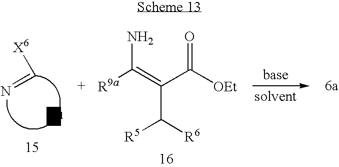

Examples of the methods illustrated in Schemes 11, 12 and 13 are described in the following literature references: M. C. Tice et al., Tetrahedron 2001, 57(14), 2689-2700; T. I. El-Emary et al., Phosphorus, Sulfur and Silicon and the Related Elements 2006, 181(11), 2459-2474; C. Mugnaini et al., Bioorganic & Medicinal Chemistry Letters 2006, 16(13), 3541-3544; P. L. Ferrarini et al., J. Heterocyclic Chem. 1983, 20, 1053-1057; A. Z. M. S. Chowdry et al., Heterocycles 2001, 55(1), 115-125.

As shown in Scheme 14, fused pyridones of Formula 6c (Formula 6 where J is J-3, X is O and Q³ is CR^{9c}) can be made by alkylating pyridones of Formula 17 with alkylating agents of formula R^{8a}—X⁷ wherein X⁷ is a halogen, mesylate or tosylate leaving group in the presence of a base such as potassium carbonate, sodium hydride or sodium methoxide and an appropriate solvent (e.g., xylenes, toluene, ethanol, tetrahydrofuran, dioxane, dichloromethane, N,N-dimethylformamide or methanol) at temperatures ranging from 0° C. to the reflux temperature of the solvent.

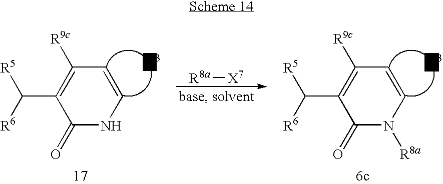

Compounds of Formula 17 can be made by the method summarized in Scheme 15. Reaction of an enamine, aniline or aminoheterocycle of Formula 18 with a ketoester of Formula 11c in the presence of a base such as potassium carbonate, sodium hydride or sodium methoxide and an appropriate solvent (e.g., xylenes, toluene, ethanol, tetrahydrofuran, dioxane, dichloromethane, N,N-dimethylformamide or methanol) at temperatures ranging from 0° C. to the reflux temperature of the solvent affords a ketoamide of Formula 19. The ketoamide of Formula 19 can then be cyclized by heating in an acidic medium such as neat polyphosphoric acid or concentrated sulfuric acid to give the corresponding compound of Formula 17. See M. Schlosser et al., Eur. J. Chem. 2004, 3714-3718 for an example of this ring formation method.

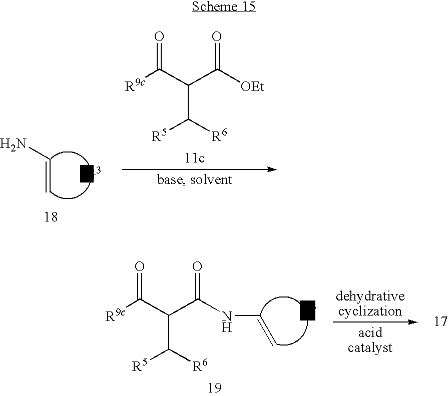

Similarly, fused pyridones of Formula 6d (Formula 6 where J is J-6, X is O and Q⁴ is CR^{9d}) can be made as illustrated in Scheme 16 by alkylating pyridones of Formula 20 with alkylating agents of formula R^{8c}—X⁸ wherein X⁸ is a halogen, mesylate or tosylate leaving group in the presence of a base such as potassium carbonate, sodium hydride or sodium methoxide and an appropriate solvent (e.g., xylenes, toluene, ethanol, tetrahydrofuran, dioxane, dichloromethane, N,N-dimethylformamide or methanol) at temperatures ranging from 0° C. to the reflux temperature of the solvent.

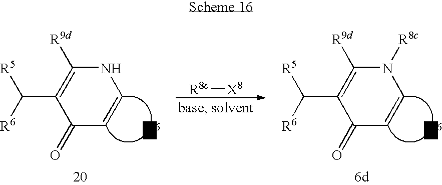

Compounds of Formula 20 can be made by the method shown in Scheme 17. Heating an enamine, aniline or aminoheterocycle of Formula 18 and a ketoester of Formula 11d with an acidic catalyst such as p-toluenesulfonic acid neat or in a solvent such as toluene, xylene or chlorobenzene gives an imine intermediate (not shown), which after isolation is then heated neat or in a solvent (e.g., diphenylether) at 150-250° C. to afford the corresponding compound of Formula 20. See J. Med. Chem. 2006, 49(21) 6351-6363 for an example of this ring formation method.

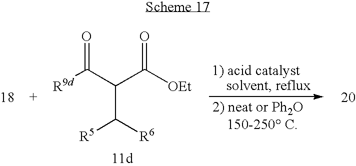

As shown in Scheme 18, fused pyridones of Formula 6e (Formula 6 where J is J-4, X is O and W¹ is CR^{10a}) can be made by alkylating pyridones of Formula 21 with alkylating agents of formula R^{8b}—X⁸ wherein X⁸ is a halogen, mesylate or tosylate leaving group in the presence of a base such as potassium carbonate, sodium hydride or sodium methoxide and an appropriate solvent (e.g., xylenes, toluene, ethanol, tetrahydrofuran, dioxane, dichloromethane, N,N-dimethylformamide or methanol) at temperatures ranging from 0° C. to the reflux temperature of the solvent.

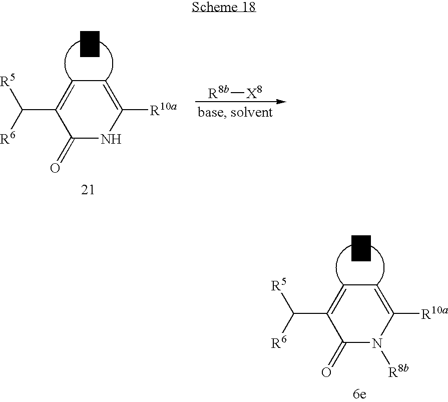

Compounds of Formula 21 can be made by the method shown in Scheme 19 involving cyclizing an α-ketoamide of Formula 22 under dehydrative conditions, preferably by heating in an acidic medium such as neat polyphosphoric acid or concentrated sulfuric acid. See PCT Patent Publication WO 2005/072132 for an example of this cyclization method.

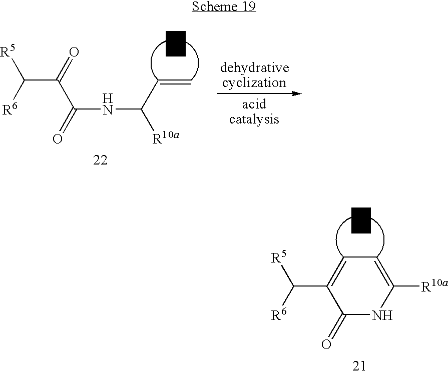

As shown in Scheme 20, a ketoamide of Formula 22 can be made by reacting an amine of Formula 23 with an α-ketoester of Formula 24 in the presence of a base such as sodium hydride or sodium methoxide and an appropriate solvent (e.g., xylenes, toluene, ethanol, tetrahydrofuran, dioxane, dichloromethane, N,N-dimethylformamide or methanol) at temperatures ranging from 0° C. to the reflux temperature of the solvent.

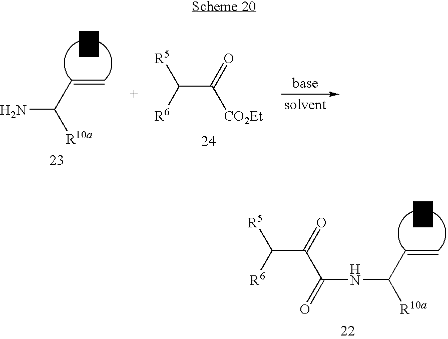

Scheme 21 illustrates the preparation of ring-fused pyrazinones of Formula 6f (Formula 6 where J is J-3, X is O and Q³ is N). A diamine of Formula 25 can be cyclized with an α-ketoester of Formula 24, preferably in a solvent such as toluene, ethanol, tetrahydrofuran, dioxane, dichloromethane, N,N-dimethylformamide or methanol, at temperatures ranging from 0° C. to the reflux temperature of the solvent to give the corresponding ring-fused pyrazinone of Formula 26. Alkylation of 26 with reagents of formula R^{8a}—X⁷ under the conditions as described for Scheme 14 provides pyrazinones of Formula 6f.

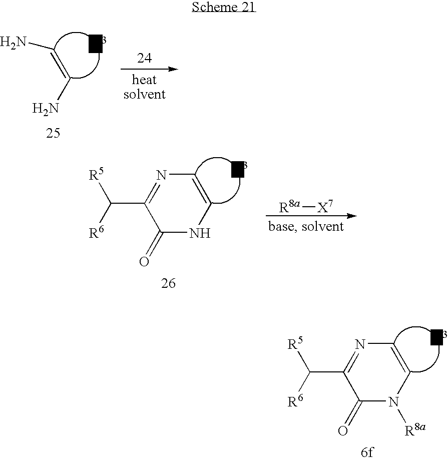

Schemes 11 through 21 illustrate methods to prepare compounds of Formula 6 having a variety of combinations of nitrogen and carbon radicals for Q¹ through Q⁴, W¹ through W², and Y¹ through Y³. Compounds of Formula 6 having other combinations of nitrogen and carbon radicals for Q¹ through Q⁴, W¹ through W², and Y¹ through Y³ can be prepared by general methods known in the art of synthetic organic chemistry, including methods analogous to those described for Schemes 11 to 21.

Compounds of Formula 1 where X is O can be converted to compounds of Formula 1 where X is S by thionation with phosphorous pentasulfide (P₂S₅) or Lawesson's reagent (4-bis(4-methoxyphenyl)-1,3,2,4-dithiadiphosphetane-2,4-disulfide) in an appropriate solvent such as toluene, pyridine, dichloromethane, tetrahydrofuran, dioxane or N,N-dimethylformamide at temperatures ranging from 0° C. to the reflux temperature of the solvent. Alternatively, intermediates of Formula 6 where X is O can be converted to corresponding intermediates of Formula 6 where X is S by use of thionation conditions just described. Compounds of Formula 6 where X is S can then be used to prepare compounds of Formula 1 where X is S by following the chemistry already described for Schemes 1-9.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, T. W. Greene, P. G. M. Wuts, Protective Groups in Organic Synthesis, 2nd Ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “m” means multiplet.

### SYNTHESIS EXAMPLE 1

### Preparation of 3-[[(4,5-dihydro-5,5-dimethyl-3-isoxazolyl)thio]methyl]-7,8-dihydro-2-(trifluoromethyl)pyrrolo[1,2-α]pyrimidin-4(6H)-one (Compound 9)

### Step A: Preparation of 3,4-dihydro-2H-pyrrol-5-amine hydrochloride (1:1)

To a stirred solution of 3,4-dihydro-5-methoxy-2H-pyrrole (5.00 g, 50.43 mmol) in ethanol (30 mL) was added ammonium chloride (2.68 g, 50.43 mmol). The reaction mixture was stirred at room temperature for 24 h. The organic layer was then concentrated under reduced pressure to afford the title product as a white solid (6.0 g), which was used without further purification in the next step.

### Step B: Preparation of 7,8-dihydro-3-methyl-2-(trifluoromethyl)pyrrolo[1,2-α]pyrimidin-4(6H)-one

To a stirred solution of 3,4-dihydro-2H-pyrrol-5-amine hydrochloride (1:1) (i.e. the product of Step A) (2.00 g, 16.6 mmol) in methanol (30 mL) was added ethyl 4,4,4-trifluoro-2-methyl-3-oxobutanoate (3.30 g, 16.6 mmol), followed by a solution of sodium methoxide (30 wt %, 8.50 g, 50.0 mmol). The reaction mixture was warmed to reflux and stirred for 24 h. Then the reaction mixture was concentrated under reduced pressure. To the residue was added water (50 mL), followed by 1 N hydrochloric acid until the pH was 2-3, and the mixture was extracted with ethyl acetate. The organic layer was dried (MgSO₄) and concentrated under reduced pressure to afford the title product as a white solid (2.3 g), which was used without further purification in the next step.

¹H NMR (CDCl₃) δ 4.18 (m, 2H), 3.16 (m, 2H), 2.23-2.29 (m, 5H).

### Step C: Preparation of 3-(bromomethyl)-7,8-dihydro-2-(trifluoromethyl)pyrrolo[1,2-α]pyrimidin-4(6H)-one

To a stirred solution of 7,8-dihydro-3-methyl-2-(trifluoromethyl)pyrrolo[1,2-α]pyrimidin-4(6H)-one (i.e. the product of Step B) (1.00 g, 4.58 mmol) in carbon tetrachloride (30 mL) was added N-bromosuccinimide (975 mg, 5.50 mmol) and 2,2′-azobis(2-methylpropionitrile) (38 mg, 0.23 mmol). The reaction mixture was heated to reflux for 2 h. The reaction mixture was cooled to room temperature and filtered, and the filtered solid was rinsed with carbon tetrachloride. The filtrate was concentrated under reduced pressure to afford the title product as an oil (1.1 g), which was used without further purification in the next step.

### Step D: Preparation of 3-[[(4,5-dihydro-5,5-dimethyl-3-isoxazolyl)thio]methyl]-7,8-dihydro-2-(trifluoromethyl)pyrrolo[1,2-α]pyrimidin-4(6H)-one

To a stirred solution of 3-(bromomethyl)-7,8-dihydro-2-(trifluoromethyl)pyrrolo[1,2-α]pyrimidin-4(6H)-one (i.e. the product of Step C) (1.10 g, 4.58 mmol) in acetonitrile (30 mL) was added 4,5-dihydro-5,5-dimethyl-3-isoxazolyl carbamimidothioate hydrochloride (1:1) (957 mg, 4.58 mmol) and potassium carbonate (1.89 g, 13.6 mmol). The reaction mixture was stirred at room temperature for 72 h. Water was added, and the mixture extracted with ethyl acetate. The organic layer was dried (MgSO₄) and concentrated under reduced pressure. The residue was purified by column chromatography (50% EtOAc, 50% hexanes) to afford the title product, a compound of the present invention, as an oil (700 mg).

¹H NMR (CDCl₃) δ 4.35 (s, 2H), 4.19 (m, 2H), 3.20 (m, 2H), 2.85 (s, 2H), 2.36 (m, 2H), 1.43 (s, 6H).

### SYNTHESIS EXAMPLE 2

### Preparation of 3-[[(4,5-dihydro-5,5-dimethyl-3-isoxazolyl)sulfonyl]methyl]-7,8-dihydro-2-(trifluoromethyl)pyrrolo[1,2-α]pyrimidin-4(6H)-one (Compound 11)

To a stirred solution of 3-[[(4,5-dihydro-5,5-dimethyl-3-isoxazolyl)thio]methyl]-7,8-dihydro-2-(trifluoromethyl)pyrrolo[1,2-c]pyrimidin-4(6H)-one (i.e. the product of Synthesis Example 1, Step D) (530 mg, 1.67 mmol) in dichloromethane (30 mL) was added 3-chloroperoxybenzoic acid (77% maximum assay, 1.5 g, 6.1 mmol). The reaction mixture was stirred at room temperature for 24 h. The mixture was diluted with dichloromethane and then washed with aqueous 5% sodium bisulfite solution and saturated aqueous sodium bicarbonate solution. The organic layer was dried (MgSO₄) and concentrated under reduced pressure. The residue was purified by column chromatography (50% EtOAc, 50% hexanes) to afford the title product, a compound of the present invention, as a white solid (430 mg).

¹H NMR (CDCl₃) δ 4.77 (s, 2H), 4.17 (m, 2H), 3.29 (m, 2H), 3.19 (s, 2H), 2.38 (m, 2H), 1.53 (s, 6H).

### SYNTHESIS EXAMPLE 3

### Preparation of 3-[[(4,5-dihydro-5,5-dimethyl-3-isoxazolyl)sulfinyl]methyl]-7,8-dihydro-2-(trifluoromethyl)pyrrolo[1,2-α]pyrimidin-4(6H)-one (Compound 10)

To a stirred solution of 3-[[(4,5-dihydro-5,5-dimethyl-3-isoxazolyl)thio]methyl]-7,8-dihydro-2-(trifluoromethyl)pyrrolo[1,2-α]pyrimidin-4(6H)-one (i.e. the product of Synthesis Example 1, Step D) (150 mg, 0.43 mmol) in a mixture of methanol and water (1:1, 6 mL) was added Oxone® potassium peroxymonosulfate (398 mg, 0.65 mmol), and the mixture was stirred at room temperature for 2 h. Water was added, and the mixture was extracted with ethyl acetate. The organic layer was dried (MgSO₄) and concentrated under reduced pressure. The residue was purified by column chromatography (75% EtOAc, 25% hexanes) to afford the title product, a compound of the present invention, as a white solid (70 mg).

¹H NMR (CDCl₃) δ 4.53 (m, 1H), 4.30 (m, 1H), 4.12 (m, 2H), 3.29 (m, 1H), 3.13-3.21 (m, 3H), 2.32 (m, 2H), 1.52 (s, 3H), 1.42 (s, 3H).

### SYNTHESIS EXAMPLE 4

### Preparation of 3-[[(4,5-dihydro-5,5-dimethyl-3-isoxazolyl)thio]methyl]-6,7,8,9-tetrahydro-2-(trifluoromethyl)-4H-pyrido[1,2-α]pyrimidin-4-one (Compound 15)

The title product, a compound of the present invention, was prepared from 3,4,5,6-tetrahydro-2-pyridinamine hydrochloride (1:1) using procedures analogous to those described for Steps B, C and D of Synthesis Example 1.

¹H NMR (CDCl₃) δ 4.23 (m, 2H), 3.90 (m, 2H), 2.90 (m, 2H), 2.77 (m, 2H), 1.82-1.97 (m, 4H), 1.33 (s, 6H).

### SYNTHESIS EXAMPLE 5

### Preparation of 3-[[(4,5-dihydro-5,5-dimethyl-3-isoxazolyl)sulfonyl]methyl]-6,7,8,9-tetrahydro-2-(trifluoromethyl)-4H-pyrido[1,2-α]pyrimidin-4-one (Compound 8)

The title product, a compound of the present invention, was prepared from 3-[[(4,5-dihydro-5,5-dimethyl-3-isoxazolyl)thio]methyl]-6,7,8,9-tetrahydro-2-(trifluoromethyl)-4H-pyrido[1,2-α]pyrimidin-4-one (i.e. the product of Example 4) using a procedure analogous to that described for Synthesis Example 2.

¹H NMR (CDCl₃) δ 4.76 (m, 2H), 3.94 (m, 2H), 3.19 (s, 2H), 3.01 (m, 2H), 1.85-2.04 (m, 4H), 1.55 (s, 6H).

### SYNTHESIS EXAMPLE 6

### Preparation of 3-[[(4,5-dihydro-5,5-dimethyl-3-isoxazolyl)thio]methyl]-1-methyl-4-(trifluoromethyl)-2(1H)-quinolinone (Compound 12)

### Step A: Preparation of 3-methyl-4-(trifluoromethyl)-2(1H)-quinolinone

To a stirred solution of aniline (1.00 g, 10.7 mmol) in toluene (100 mL) was added ethyl 4,4,4-trifluoro-2-methyl-3-oxobutanoate (2.55 g, 12.9 mmol), followed by sulfuric acid (25 mL). The reaction mixture was heated to 100° C. for 24 h. Then the reaction mixture was cooled to room temperature and poured in water (100 mL). Aqueous 6 N sodium hydroxide was added until the pH was ˜6, and the mixture was extracted with ethyl acetate. The organic layer was dried (MgSO₄) and concentrated under reduced pressure. The residue was purified by column chromatography (30% EtOAc, 70% hexanes) to afford the title product as a white solid (160 mg).

¹H NMR (CDCl₃) δ 7.89 (m, 1H), 7.53 (m, 1H), 7.40 (m, 1H), 7.28 (m, 1H), 2.54 (m, 3H).

### Step B: Preparation of 1,3-dimethyl-4-(trifluoromethyl)-2(1H)-quinolinone

To a stirred solution of 3-methyl-4-(trifluoromethyl)-2(1H)-quinolinone (i.e. the product of Step A) (140 g, 0.6 mmol) in N,N-dimethylformamide (10 mL) was added potassium carbonate (330 mg, 2.4 mmol) and iodomethane (0.12 mL, 2.4 mmol). The reaction mixture was stirred at room temperature for 24 h. Water was added, and the mixture extracted with ethyl acetate. The organic layer was dried (MgSO₄) and concentrated under reduced pressure. The residue was purified by column chromatography (20% EtOAc, 80% hexanes) to afford the title product as a white solid (140 mg).

¹H NMR (CDCl₃) δ 7.94 (m, 1H), 7.58 (m, 1H), 7.42 (m, 1H), 7.29 (m, 1H), 3.79 (s, 3H), 2.52 (m, 3H).

### Step C: Preparation of 3-[[(4,5-dihydro-5,5-dimethyl-3-isoxazolyl)thio]methyl]-1-methyl-4-(trifluoromethyl)-2(1H)-quinolinone

The title product, a compound of the present invention, was prepared from 1,3-dimethyl-4-(trifluoromethyl)-2(1H)-quinolinone (i.e. the product of Step B) using procedures analogous to those described for Steps C and D of Synthesis Example 1.

¹H NMR (CDCl₃) δ 7.959 (m, 1H), 7.64 (m, 1H), 7.45 (m, 1H), 7.33 (m, 1H), 4.65 (m, 2H), 3.80 (s, 3H), 2.86 (s, 2H), 1.43 (s, 6H).

### SYNTHESIS EXAMPLE 7

### Preparation of 3-[[(4,5-dihydro-5,5-dimethyl-3-isoxazolyl)sulfonyl]methyl]-1-methyl-4-(trifluoromethyl)-2(1H)-quinolinone (Compound 1)

The title product, a compound of the present invention, was prepared from 3-[[(4,5-dihydro-5,5-dimethyl-3-isoxazolyl)thio]methyl]-1-methyl-4-(trifluoromethyl)-2(1H)-quinolinone (i.e. the product of Synthesis Example 6, Step C) using a procedure analogous to that described for Synthesis Example 2.

¹H NMR (CDCl₃) δ 8.09 (m, 1H), 7.69 (m, 1H), 7.45 (m, 1H), 7.36 (m, 1H), 5.16 (m, 2H), 3.76 (s, 3H), 3.24 (s, 2H), 1.55 (s, 6H)

### SYNTHESIS EXAMPLE 8

### Preparation of 6-[[(4,5-dihydro-5,5-dimethyl-3-isoxazolyl)thio]methyl]-2,3-dihydro-7-(trifluoromethyl)-5H-oxazolo[3,2-α]pyrimidin-5-one (Compound 13)

### Step A: Preparation of 5-methyl-6-(trifluoromethyl)-2,4(1H,3H)-pyrimidinedione

To a stirred solution of urea (2.27 g, 37 mmol) in methanol (50 mL) was added ethyl 4,4,4-trifluoro-2-methyl-3-oxobutanoate (5.00 g, 25 mmol), followed by a methanol solution of sodium methoxide (30 wt %, 68 g, 37 mmol). The reaction mixture was warmed to reflux and stirred for 24 h. Then the reaction mixture was concentrated under reduced pressure. To the residue was added water (50 mL), followed by 1 N hydrochloric acid until the pH was 2-3, and the mixture was extracted with ethyl acetate. The organic layer was dried (MgSO₄) and concentrated under reduced pressure to afford the title product as a white solid (1.9 g), which was used without further purification in the next step.

¹H NMR ((CD₃)₂SO) δ 11.61 (s, 1H), 11.42 (s, 1H), 1.90 (m, 3H).

### Step B: Preparation of 2,3-dihydro-6-methyl-7-(trifluoromethyl)-5H-oxazolo-[3,2-α]pyrimidin-5-one

To a stirred solution of 5-methyl-6-(trifluoromethyl)-2,4(1H,3H)-pyrimidinedione (i.e. the product of Step A) (683 mg, 0.35 mmol) in N,N-dimethylformamide (20 mL) was added potassium carbonate (2.43 g, 17.6 mmol) and 1,2-dibromoethane (0.6 mL, 7.0 mmol). The reaction mixture was warmed to 80° C. and stirred for 24 h. Water was added, and the mixture extracted with ethyl acetate. The organic layer was dried (MgSO₄) and concentrated under reduced pressure. The residue was purified by column chromatography (50% EtOAc, 50% hexanes) to afford the title product as a white solid (900 mg).

¹H NMR (CDCl₃) δ 4.80 (m, 2H), 4.34 (m, 2H), 2.18 (m, 3H).

### Step C: Preparation of 6-[[(4,5-dihydro-5,5-dimethyl-3-isoxazolyl)thio]methyl]-2,3-dihydro-7-(trifluoromethyl)-5H-oxazolo[3,2-α]pyrimidin-5-one

The title product, a compound of the present invention, was prepared from 2,3-dihydro-6-methyl-7-(trifluoromethyl)-5H-oxazolo[3,2-α]pyrimidin-5-one (i.e. the product of Step B) using procedures analogous to those described for Steps C and D of Synthesis Example 1.

¹H NMR (CDCl₃) δ 4.85 (m, 2H), 4.38 (m, 2H), 4.32 (s, 2H), 2.84 (s, 2H), 1.43 (s, 6H).

### SYNTHESIS EXAMPLE 9

### Preparation of 6-[[(4,5-dihydro-5,5-dimethyl-3-isoxazolyl)sulfonyl]methyl]-2,3-dihydro-7-(trifluoromethyl)-5H-oxazolo[3,2-α]pyrimidin-5-one (Compound 2)

The title product, a compound of the present invention, was prepared from 6-[[(4,5-dihydro-5,5-dimethyl-3-isoxazolyl)thio]methyl]-2,3-dihydro-7-(trifluoromethyl)-5H-oxazolo[3,2-α]pyrimidin-5-one (i.e. the product of Synthesis Example 8, Step C) using a procedure analogous to that described for Synthesis Example 2.

¹H NMR ((CD₃)₂SO) δ 5.03 (m, 2H), 4.68 (m, 2H), 3.23 (s, 2H) 1.51 (s, 6H).

### SYNTHESIS EXAMPLE 10

### Preparation of 3-[[(4,5-dihydro-5,5-dimethyl-3-isoxazolyl)thio]methyl]-2-(trifluoromethyl)-4H-pyrido[1,2-α]pyrimidin-4-one (Compound 14)

### Step A: Preparation of 3-methyl-2-(trifluoromethyl)-4H-pyrido[1,2-α]pyrimidin-4-one

To a stirred solution of 2-aminopyridine (2.18 g, 23.1 mmol) in a polyphosphoric acid (14.5 g) heated to 80° C. was added ethyl 4,4,4-trifluoro-2-methyl-3-oxobutanoate (5.51 g, 27.8 mmol). The reaction mixture was then warmed to 110° C. for 5 h. The mixture was cooled to 60° C. and then poured slowly into ice water. The mixture was neutralized to pH 7 by addition of aqueous 50% sodium hydroxide and then extracted with ethyl acetate. The organic layer was dried (MgSO₄) and concentrated under reduced pressure. The residue was purified by column chromatography (50% EtOAc, 50% hexanes) to afford the title product as a white solid (110 mg).

¹H NMR (CDCl₃) δ 9.05 (m, 1H), 7.75 (m, 2H), 7.21 (m, 1H), 2.42 (m, 3H).

### Step B: Preparation of 3-[[(4,5-dihydro-5,5-dimethyl-3-isoxazolyl)thio]methyl]-2-(trifluoromethyl)-4H-pyrido[1,2-α]pyrimidin-4-one

The title product, a compound of the present invention, was prepared from 3-methyl-2-(trifluoromethyl)-4H-pyrido[1,2-α]pyrimidin-4-one (i.e. the product of Step A) using procedures analogous to those described by Steps C and D of Synthesis Example 1.

¹H NMR (CDCl₃) δ 9.05 (m, 1H), 7.91 (m, 1H), 7.82 (m, 1H), 7.32 (m, 1H), 4.55 (s, 2H), 2.88 (s, 2H), 1.45 (s, 6H).

### SYNTHESIS EXAMPLE 11

### Preparation of 3-[[(4,5-dihydro-5,5-dimethyl-3-isoxazolyl)sulfonyl]methyl-2-(trifluoromethyl)-4H-pyrido[1,2-α]pyrimidin-4-one (Compound 7)

The title product, a compound of the present invention, was prepared from 3-[[(4,5-dihydro-5,5-dimethyl-3-isoxazolyl)thio]methyl]-2-(trifluoromethyl)-4H-pyrido[1,2-α]pyrimidin-4-one (i.e. the product of Synthesis Example 10, Step B) using a procedure analogous to that described for Synthesis Example 2.

¹H NMR (CDCl₃) δ 9.05 (m, 1H), 7.99 (m, 1H), 7.87 (m, 1H), 7.39 (m, 1H), 4.95 (m, 2H), 3.23 (s, 2H), 1.56 (s, 6H).

By the procedures described herein together with methods known in the art, the following compounds of Tables 1 to 4 can be prepared.

TABLE 1

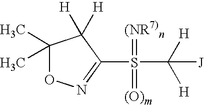

m is 2; n is 0; and
J is as tabulated below:

| | |
|---|---|
| 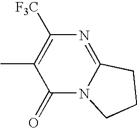 | 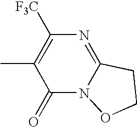 |
| 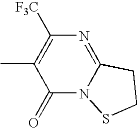 | 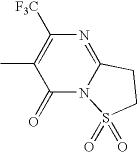 |
| 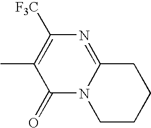 | 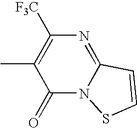 |
| 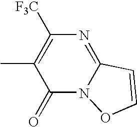 | 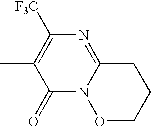 |
| 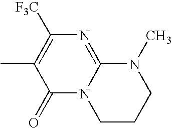 | 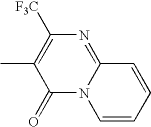 |
| 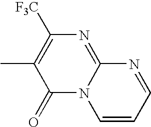 | 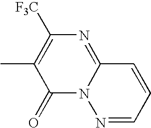 |
| 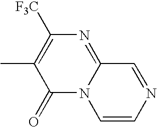 | 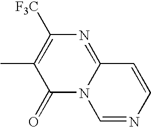 |
| 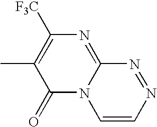 | 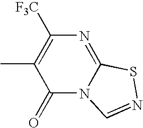 |
| 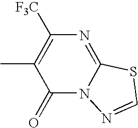 | 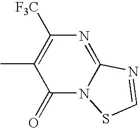 |
| 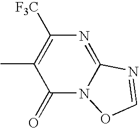 | 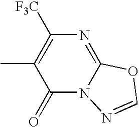 |
| 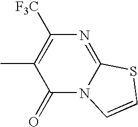 | 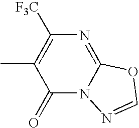 |
| 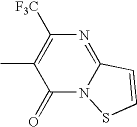 | 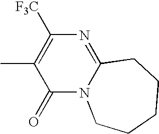 |
| 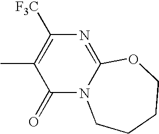 | 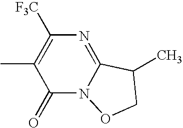 |
| 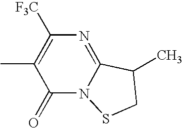 | 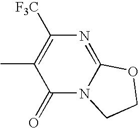 |
| 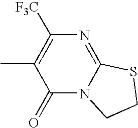 | 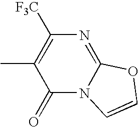 |
| 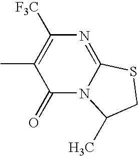 | 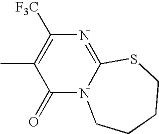 |
| 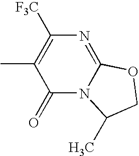 | 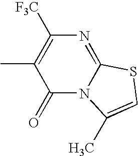 |
| 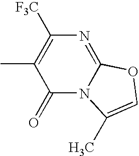 | 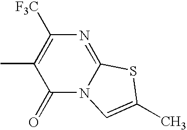 |
| 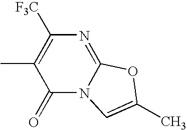 | 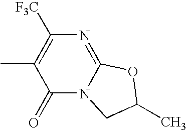 |
| 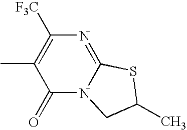 | 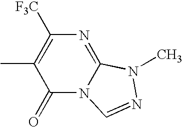 |
| 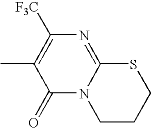 | 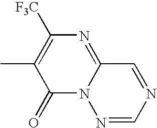 |
| 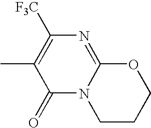 | 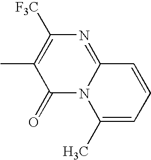 |
| 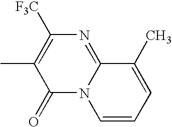 | 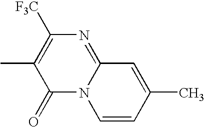 |
| 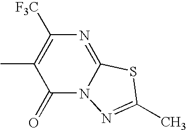 | 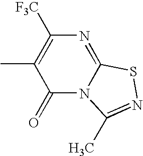 |
| 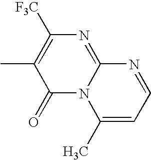 | 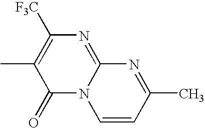 |
| 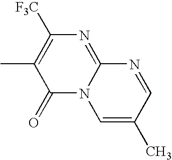 | 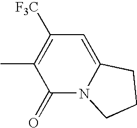 |
| 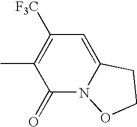 | 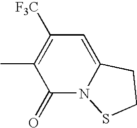 |
| 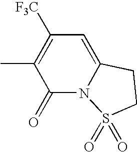 | 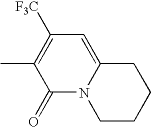 |
| 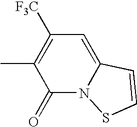 | 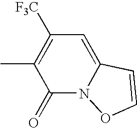 |
| 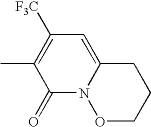 | 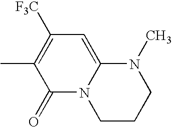 |
| 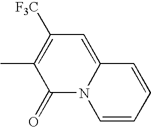 | 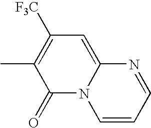 |
| 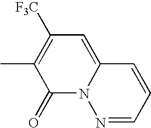 | 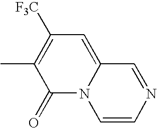 |
| 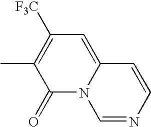 | 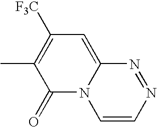 |
| 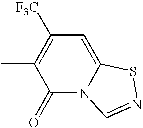 | 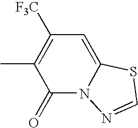 |
| 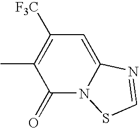 | 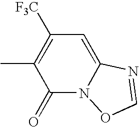 |
| 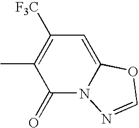 | 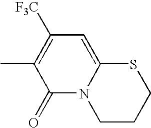 |
| 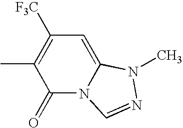 | 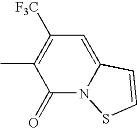 |
| 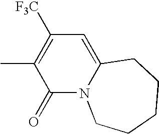 | 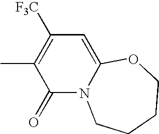 |
| 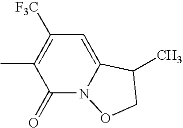 | 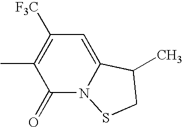 |
| 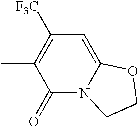 | 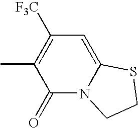 |
| 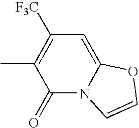 | 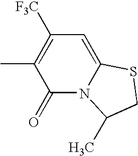 |
| 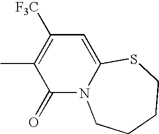 | 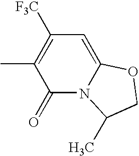 |
| 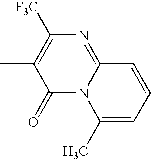 | 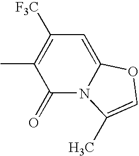 |
| 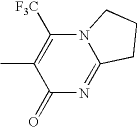 | 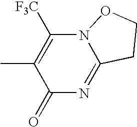 |
| 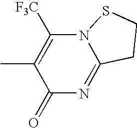 | 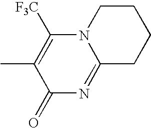 |
| 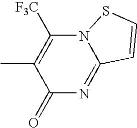 | 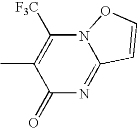 |
| 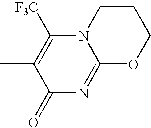 | 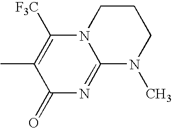 |
| 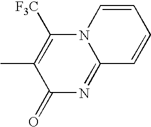 | 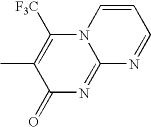 |
| 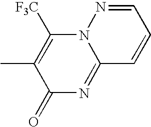 | 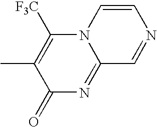 |
| 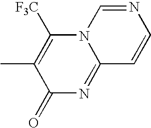 | 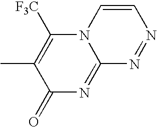 |
| 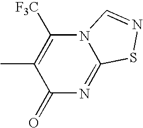 | 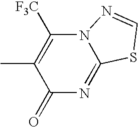 |
| 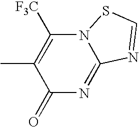 | 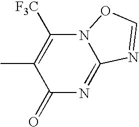 |
| 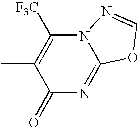 | 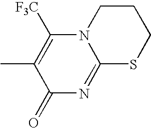 |
| 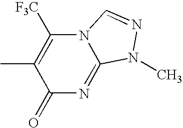 | 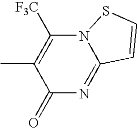 |
| 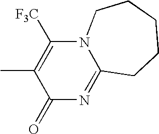 | 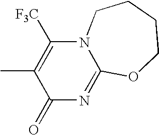 |
| 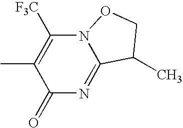 | 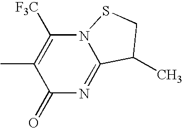 |
| 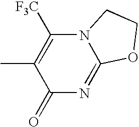 | 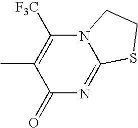 |
| 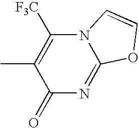 | 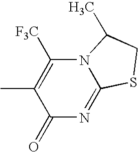 |
| 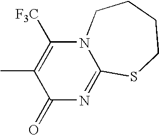 | 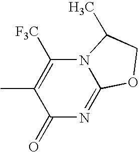 |
| 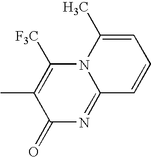 | 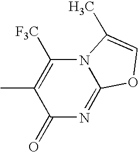 |
| 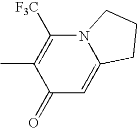 | 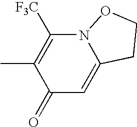 |
| 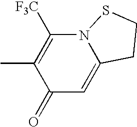 | 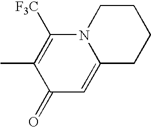 |
| 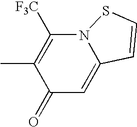 | 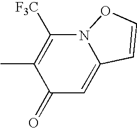 |
| 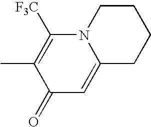 | 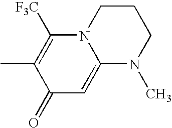 |
| 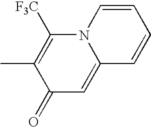 | 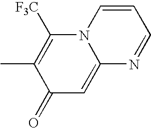 |
| 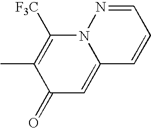 | 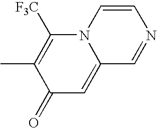 |
| 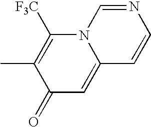 | 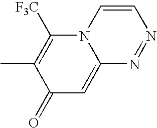 |
| 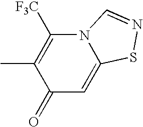 | 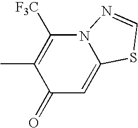 |
| 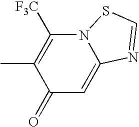 | 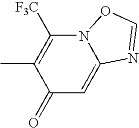 |
| 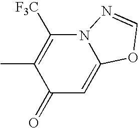 | 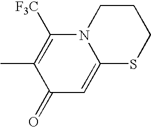 |
| 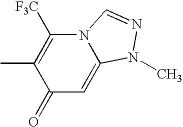 | 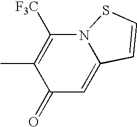 |
| 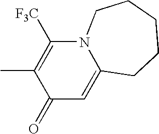 | 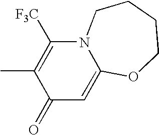 |
| 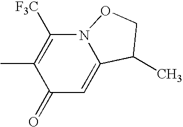 | 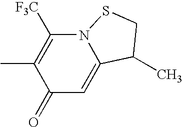 |
| 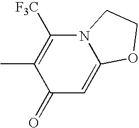 | 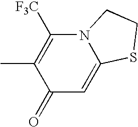 |
| 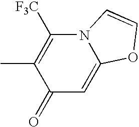 | 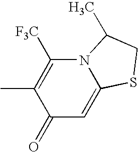 |
| 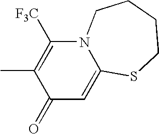 | 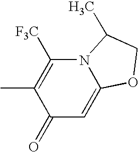 |
| 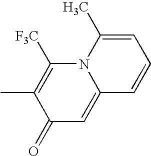 | 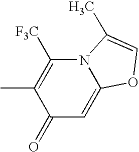 |
| 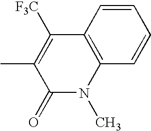 | 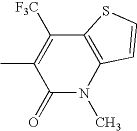 |
| 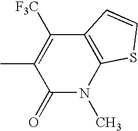 | 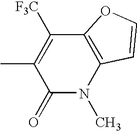 |
| 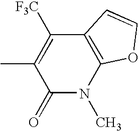 | 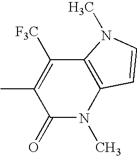 |
| 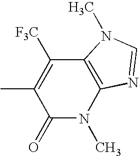 | 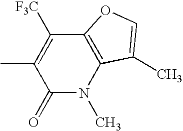 |
| 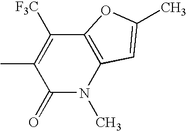 | 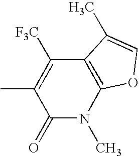 |
| 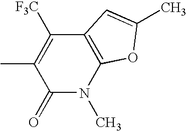 | 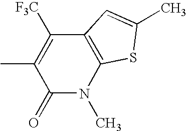 |
| 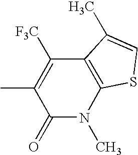 | 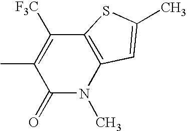 |
| 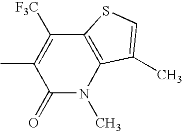 | 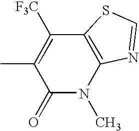 |
| 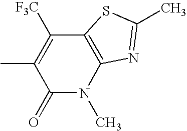 | 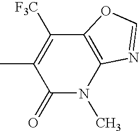 |
| 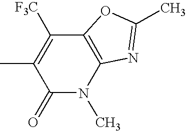 | 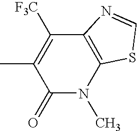 |
| 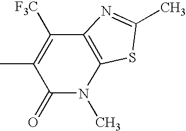 | 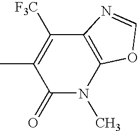 |
| 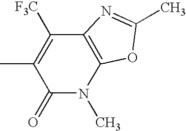 | 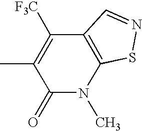 |
| 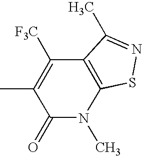 | 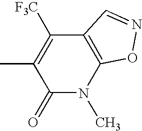 |
| 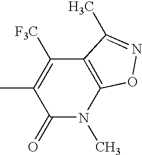 | 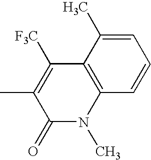 |
| 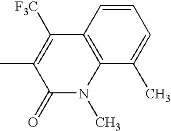 | 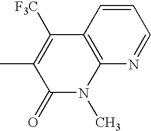 |
| 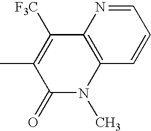 | 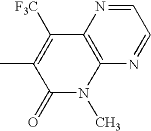 |
| 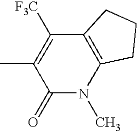 | 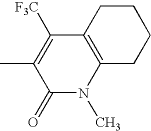 |
| 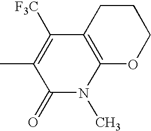 | 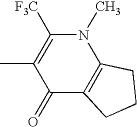 |
| 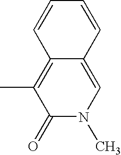 | 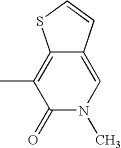 |
| 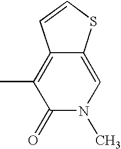 | 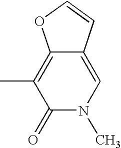 |
| 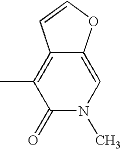 | 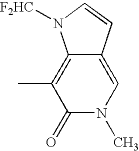 |
| 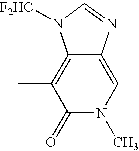 | 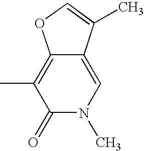 |
| 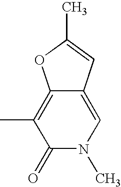 | 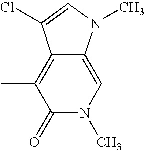 |
| 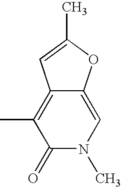 | 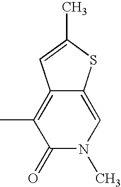 |
| 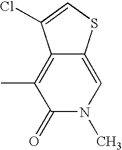 | 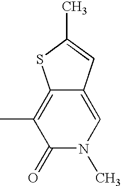 |
| 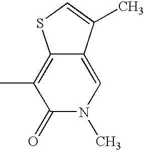 | 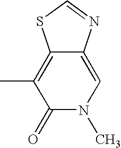 |
| 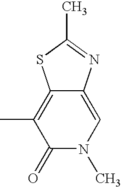 | 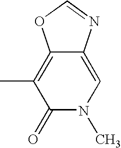 |
| 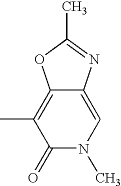 | 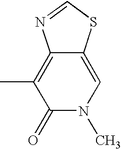 |
| 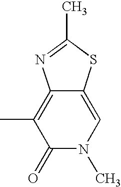 | 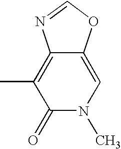 |
| 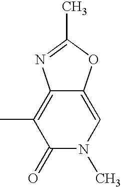 | 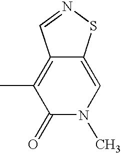 |
| 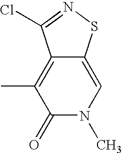 | 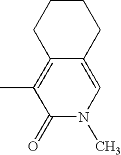 |
| 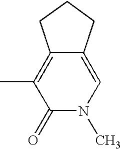 | 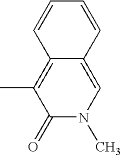 |
| 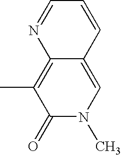 | 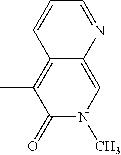 |
| 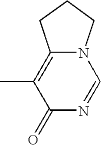 | 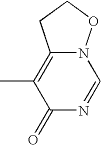 |
| 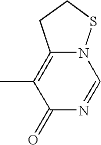 | 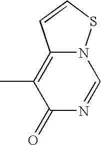 |
| 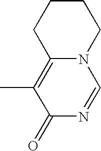 | 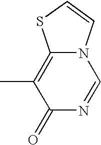 |
| 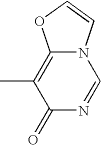 | 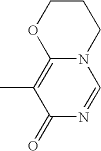 |
| 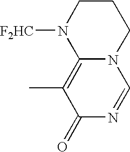 | 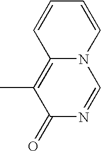 |
| 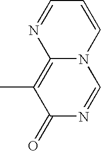 | 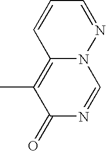 |
| 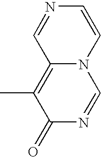 | 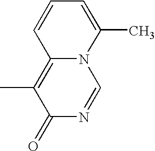 |
| 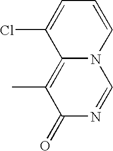 | 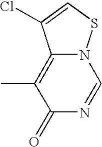 |
| 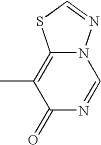 | 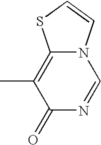 |
| 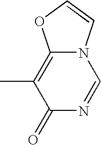 | 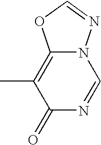 |
| 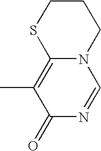 | 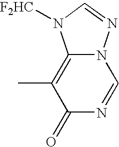 |
| 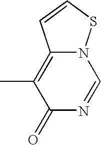 | 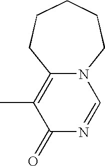 |
| 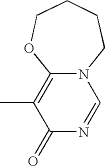 | 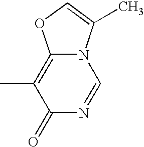 |
| 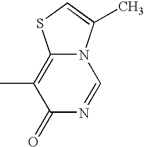 | 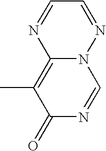 |
| 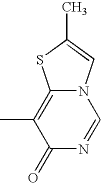 | 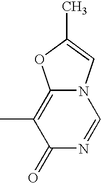 |
| 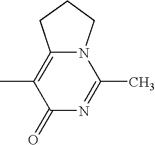 | 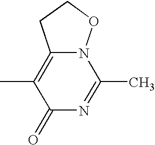 |
| 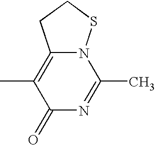 | 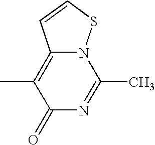 |
| 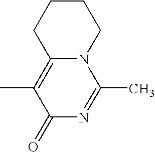 | 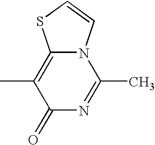 |
| 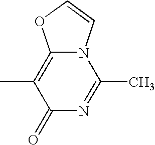 | 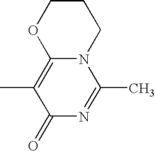 |
| 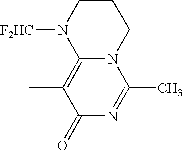 | 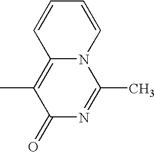 |
| 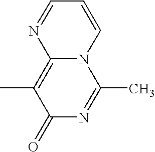 | 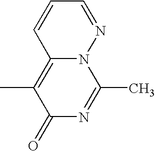 |
| 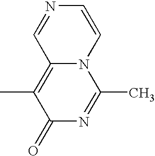 | 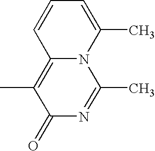 |
| 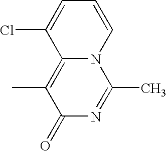 | 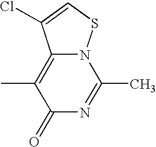 |
| 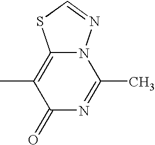 | 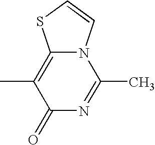 |
| 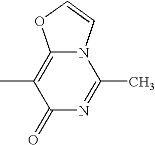 | 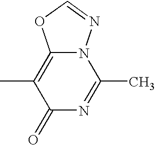 |
| 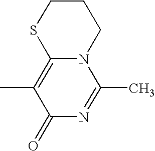 | 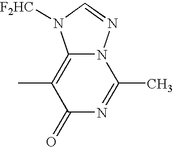 |
| 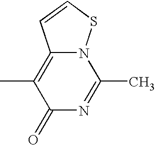 | 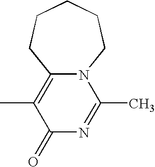 |
| 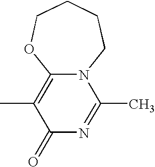 | 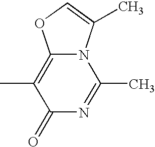 |
| 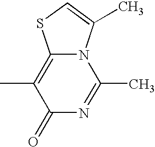 | 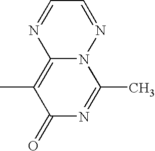 |
| 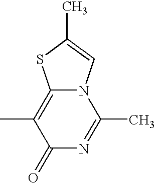 | 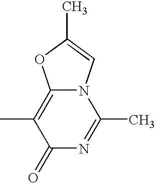 |
| 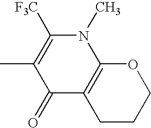 | 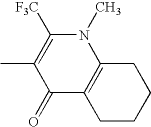 |
| 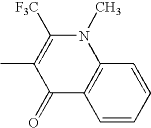 | 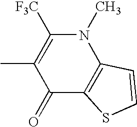 |
| 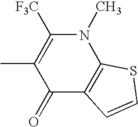 | 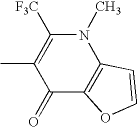 |
| 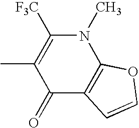 | 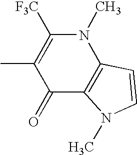 |
| 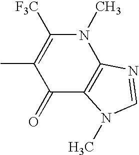 | 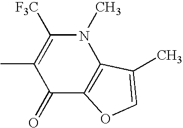 |
| 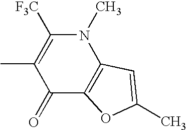 | 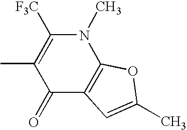 |
| 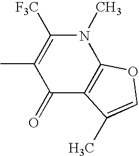 | 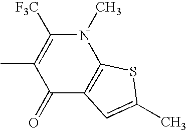 |
| 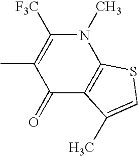 | 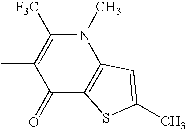 |
| 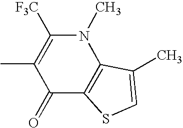 | 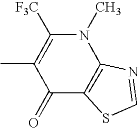 |
| 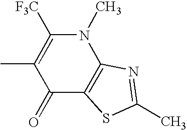 | 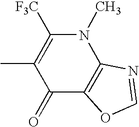 |
| 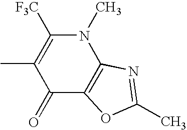 | 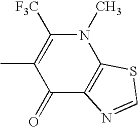 |
| 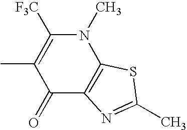 | 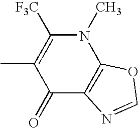 |
| 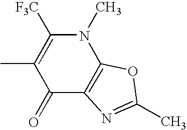 | 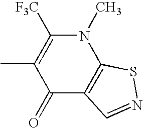 |
| 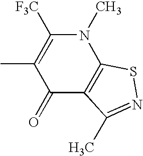 | 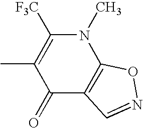 |
| 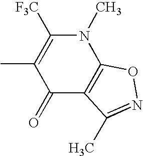 | 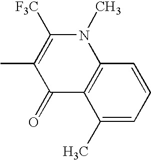 |
| 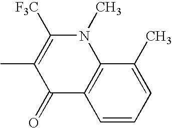 | 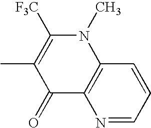 |
| 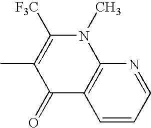 | 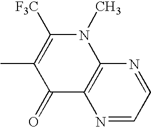 |

Also disclosed is TABLE 2, which is constructed the same as TABLE 1 except that m is 1 instead of 2. Also disclosed is TABLE 3, which is constructed the same as TABLE 1 except that m is 0 instead of 2. Also disclosed is TABLE 4, which is constructed the same as TABLE 1 except that m is 1 instead of 2, n is 1 instead of 0, and R⁷ is H.

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion.

The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g, oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A-D. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 6 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 7 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 8 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 4 | 1.0% |
| polyoxyethylene sorbitol hexoleate | 23.0% |
| C₆-C₁₀ fatty acid methyl ester | 76.0% |

### Example F

Microemulsion

| | |
|---|---|
| Compound 11 | 1.0% |
| polyvinylpyrrolidone-vinyl acetate copolymer | 30.0% |
| alkylpolyglycoside | 30.0% |
| glyceryl monooleate | 19.0% |
| water | 20.0% |

Test results indicate that the compounds of Formula 1 wherein the sum of n+m is greater than zero are highly active preemergent and/or postemergent herbicides and/or plant growth regulants. Compounds of Formula 1 wherein the sum of n+m is zero (i.e. sulfides) may show slight herbicidal activity at commercially desirable application rates, but the primary utility of these compounds is as key intermediates for the preparation of highly herbicidally active compounds of Formula 1 wherein the sum of n+m is greater than zero (e.g., by the methods of Schemes 1 through 4). Therefore the following biological disclosure relating to compounds of Formula 1 and compounds of this invention pertains particularly to compounds wherein the sum of n+m is greater than zero.

These compounds generally show highest activity for preemergence weed control (i.e. applied before weed seedlings emerge from the soil) and early postemergence weed control (i.e. applied when the emerged weed seedlings are still young). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of the invention are particularly useful for selective control of weeds in crops of corn, rice (both upland and paddy), soybeans and wheat. Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation.

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.0001 to 20 kg/ha with a typical range of about 0.001 to 5 kg/ha and a more typical range of about 0.004 to 3 kg/ha. One skilled in the art can easily determine the herbicidally effective amount necessary for the desired level of weed control.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a herbicidally effective amount of a compound of Formula 1 and a biologically effective amount of at least one additional biologically active compound or agent and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminocyclopyrachlor and its methyl and ethyl esters and its sodium and potassium salts, aminopyralid, aminotriazole, amitrole, ammonium sulfamate, anilofos, asulam, atrazine, azimsulfuron, beflubutamid, benazolin, benazolin-ethyl, bencarbazone, benfluralin, benfuresate, bensulfuron-methyl, bensulide, bentazone, benzobicyclon, benzofenap, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clefoxydim, clethodim, clodinafop-propargyl, clomazone, clomeprop, clopyralid, clopyralid-olamine, cloransulam-methyl, cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, flucarbazone, flucetosulfuron, fluchloralin, flufenacet, flufenpyr, flufenpyr-ethyl, fluazolate, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, fluorochloridone, fluroxypyr, flurtamone, fluthiacet-methyl, fomesafen, foramsulfuron, fosamine-ammonium, glufosinate, glufosinate-ammonium, glyphosate and its salts such as ammonium, isopropylammonium, potassium, sodium (including sesquisodium) and trimesium (alternatively named sulfosate), halosulfuron-methyl, haloxyfop-etotyl, haloxyfop-methyl, hexazinone, imazamethabenz-methyl, imazamox, imazapic, imazapyr, imazaquin, imazaquin-ammonium, imazethapyr, imazethapyr-ammonium, imazosulfuron, indanofan, iodosulfuron-methyl, ioxynil, ioxynil octanoate, ioxynil-sodium, isoproturon, isouron, isoxaben, isoxaflutole, isoxachlortole, lactofen, lenacil, linuron, maleic hydrazide, MCPA and its dimethylammonium, potassium and sodium salts, MCPA-isoctyl, MCPA-thioethyl, MCPB and its sodium salt, MCPB-ethyl, mecoprop, mecoprop-P, mefenacet, mefluidide, mesosulfuron-methyl, mesotrione, metam-sodium, metamifop, metamitron, metazachlor, methabenzthiazuron, methylarsonic acid and its calcium, monoammonium, monosodium and disodium salts, methyldymron, metobenzuron, metobromuron, metolachlor, S-metholachlor, metosulam, metoxuron, metribuzin, metsulfuron-methyl, molinate, monolinuron, naproanilide, napropamide, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, phenmedipham, picloram, picloram-potassium, picolinafen, pinoxaden, piperophos, pretilachlor, primisulfuron-methyl, prodiamine, profoxydim, prometon, prometryn, pronamide, propachlor, propanil, propaquizafop, propazine, propham, propisochlor, propoxycarbazone, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimsulfuron, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tembotrione, tepraloxydim, terbacil, terbumeton, terbuthylazine, terbutryn, thenylchlor, tefuryltrione, thiazopyr, thiencarbazone, thifensulfuron-methyl, thiobencarb, tiocarbazil, topramezone, tralkoxydim, tri-allate, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butyl.) Butyl. and Puccinia thlaspeos Schub.

In certain instances, combinations of a compound of this invention with other biologically active (particularly herbicidal) compounds or agents (i.e. active ingredients) can result in a greater-than-additive (i.e. synergistic) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When synergism of herbicidal active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of Formula 1 with at least one other herbicidal active ingredient. Of particular note is such a combination where the other herbicidal active ingredient has a different site of action from the compound of Formula 1. In certain instances, a combination with at least one other herbicidal active ingredient having a similar spectrum of control but a different site of action will be particularly advantageous for resistance management. Thus, a composition of the present invention can further comprise a biologically effective amount of at least one additional herbicidal active ingredient having a similar spectrum of control but a different site of action. Herbicidally effective amounts of compounds of the invention as well as herbicidally effective amounts of other herbicides can be easily determined by one skilled in the art through simple experimentation.

Preferred for better control of undesired vegetation (e.g., lower use rate, broader spectrum of weeds controlled, or enhanced crop safety) or for preventing the development of resistant weeds are mixtures of a compound of this invention with a herbicide selected from the group consisting of 2,4-D, aminocyclopyrachlor, aminopyralid, atrazine, bromoxynil, bromoxynil octanoate, carfentrazone-ethyl, chlorimuron-ethyl, chlorsulfuron, clomazone, clopyralid, clopyralid-olamine, dicamba and its diglycolammonium, dimethylammonium, potassium and sodium salts, diflufenican, dimethenamid, dimethenamid-P, diuron, florasulam, flufenacet, flumetsulam, flumioxazin, flupyrsulfuron-methyl, flupyrsulfuron-methyl-sodium, fluoroxypyr, glyphosate (particularly glyphosate-isopropylammonium, glyphosate-sodium, glyphosate-potassium, glyphosate-trimesium), hexazinone, imazamethabenz-methyl, imazaquin, imazethapyr, iodosulfuron-methyl, isoproturon, lactofen, MCPA and its dimethylammonium, potassium and sodium salts, MCPA-isoctyl, MCPA-thioethyl, mesosulfuron-methyl, mesotrione, metribuzin, metsulfuron-methyl, nicosulfuron, oxyfluorfen, pendimethalin, pinoxaden, pronamide, prosulfuron, pyrasulfotole, pyroxsulam, quinclorac, rimsulfuron, S-metolachlor, sulfentrazone, thifensulfuron-methyl, triasulfuron, tribenuron-methyl, triclopyr, triclopyr-butotyl, and triclopyr-triethylammonium.

Specifically preferred mixtures (compound numbers refer to compounds in Index Tables A-G) are selected from the group: compound 2 and 2,4-D; compound 8 and 2,4-D; compound 21 and 2,4-D; compound 22 and 2,4-D; compound 2 and aminocyclopyrachlor; compound 8 and aminocyclopyrachlor; compound 21 and aminocyclopyrachlor; compound 22 and aminocyclopyrachlor; compound 2 and aminopyralid; compound 8 and aminopyralid; compound 21 and aminopyralid; compound 22 and aminopyralid; compound 2 and atrazine; compound 8 and atrazine; compound 21 and atrazine; compound 22 and atrazine; compound 2 and bromoxynil; compound 8 and bromoxynil; compound 21 and bromoxynil; compound 22 and bromoxynil; compound 2 and bromoxynil octanoate; compound 8 and bromoxynil octanoate; compound 21 and bromoxynil octanoate; compound 22 and bromoxynil octanoate; compound 2 and carfentrazone-ethyl; compound 8 and carfentrazone-ethyl; compound 21 and carfentrazone-ethyl; compound 22 and carfentrazone-ethyl; compound 2 and chlorimuron-ethyl; compound 8 and chlorimuron-ethyl; compound 21 and chlorimuron-ethyl; compound 22 and chlorimuron-ethyl; compound 2 and chlorsulfuron; compound 8 and chlorsulfuron; compound 21 and chlorsulfuron; compound 22 and chlorsulfuron; compound 2 and clomazone; compound 8 and clomazone; compound 21 and clomazone; compound 22 and clomazone; compound 2 and clopyralid; compound 8 and clopyralid; compound 21 and clopyralid; compound 22 and clopyralid; compound 2 and clopyralid-olamine; compound 8 and clopyralid-olamine; compound 21 and clopyralid-olamine; compound 22 and clopyralid-olamine; compound 2 and dicamba; compound 8 and dicamba; compound 21 and dicamba; compound 22 and dicamba; compound 2 and diflufenican; compound 8 and diflufenican; compound 21 and diflufenican; compound 22 and diflufenican; compound 2 and dimethenamid; compound 8 and dimethenamid; compound 21 and dimethenamid; compound 22 and dimethenamid; compound 2 and dimethenamid-P; compound 8 and dimethenamid-P; compound 21 and dimethenamid-P; compound 22 and dimethenamid-P; compound 2 and diuron; compound 8 and diuron; compound 21 and diuron; compound 22 and diuron; compound 2 and florasulam; compound 8 and florasulam; compound 21 and florasulam; compound 22 and florasulam; compound 2 and flufenacet; compound 8 and flufenacet; compound 21 and flufenacet; compound 22 and flufenacet; compound 2 and flumetsulam; compound 8 and flumetsulam; compound 21 and flumetsulam; compound 22 and flumetsulam; compound 2 and flumioxazin; compound 8 and flumioxazin; compound 21 and flumioxazin; compound 22 and flumioxazin; compound 2 and flupyrsulfuron-methyl; compound 8 and flupyrsulfuron-methyl; compound 21 and flupyrsulfuron-methyl; compound 22 and flupyrsulfuron-methyl; compound 2 and flupyrsulfuron-methyl-sodium; compound 8 and flupyrsulfuron-methyl-sodium; compound 21 and flupyrsulfuron-methyl-sodium; compound 22 and flupyrsulfuron-methyl-sodium; compound 2 and fluoroxypyr; compound 8 and fluoroxypyr; compound 21 and fluoroxypyr; compound 22 and fluoroxypyr; compound 2 and glyphosate; compound 8 and glyphosate; compound 21 and glyphosate; compound 22 and glyphosate; compound 2 and hexazinone; compound 8 and hexazinone; compound 21 and hexazinone; compound 22 and hexazinone; compound 2 and imazamethabenz-methyl; compound 8 and imazamethabenz-methyl; compound 21 and imazamethabenz-methyl; compound 22 and imazamethabenz-methyl; compound 2 and imazaquin; compound 8 and imazaquin; compound 21 and imazaquin; compound 22 and imazaquin; compound 2 and imazethapyr; compound 8 and imazethapyr; compound 21 and imazethapyr; compound 22 and imazethapyr; compound 2 and iodosulfuron-methyl; compound 8 and iodosulfuron-methyl; compound 21 and iodosulfuron-methyl; compound 22 and iodosulfuron-methyl; compound 2 and isoproturon; compound 8 and isoproturon; compound 21 and isoproturon; compound 22 and isoproturon; compound 2 and lactofen; compound 8 and lactofen; compound 21 and lactofen; compound 22 and lactofen; compound 2 and MCPA; compound 8 and MCPA; compound 21 and MCPA; compound 22 and MCPA; compound 2 and MCPA-isoctyl; compound 8 and MCPA-isoctyl; compound 21 and MCPA-isoctyl; compound 22 and MCPA-isoctyl; compound 2 and MCPA-thioethyl; compound 8 and MCPA-thioethyl; compound 21 and MCPA-thioethyl; compound 22 and MCPA-thioethyl; compound 2 and mesosulfuron-methyl; compound 8 and mesosulfuron-methyl; compound 21 and mesosulfuron-methyl; compound 22 and mesosulfuron-methyl; compound 2 and mesotrione; compound 8 and mesotrione; compound 21 and mesotrione; compound 22 and mesotrione; compound 2 and metribuzin; compound 8 and metribuzin; compound 21 and metribuzin; compound 22 and metribuzin; compound 2 and metsulfuron-methyl; compound 8 and metsulfuron-methyl; compound 21 and metsulfuron-methyl; compound 22 and metsulfuron-methyl; compound 2 and nicosulfuron; compound 8 and nicosulfuron; compound 21 and nicosulfuron; compound 22 and nicosulfuron; compound 2 and oxyfluorfen; compound 8 and oxyfluorfen; compound 21 and oxyfluorfen; compound 22 and oxyfluorfen; compound 2 and pendimethalin; compound 8 and pendimethalin; compound 21 and pendimethalin; compound 22 and pendimethalin; compound 2 and pinoxaden; compound 8 and pinoxaden; compound 21 and pinoxaden; compound 22 and pinoxaden; compound 2 and pronamide; compound 8 and pronamide; compound 21 and pronamide; compound 22 and pronamide; compound 2 and prosulfuron; compound 8 and prosulfuron; compound 21 and prosulfuron; compound 22 and prosulfuron; compound 2 and pyrasulfotole; compound 8 and pyrasulfotole; compound 21 and pyrasulfotole; compound 22 and pyrasulfotole; compound 2 and pyroxsulam; compound 8 and pyroxsulam; compound 21 and pyroxsulam; compound 22 and pyroxsulam; compound 2 and quinclorac; compound 8 and quinclorac; compound 21 and quinclorac; compound 22 and quinclorac; compound 2 and rimsulfuron; compound 8 and rimsulfuron; compound 21 and rimsulfuron; compound 22 and rimsulfuron; compound 2 and S-metolachlor; compound 8 and S-metolachlor; compound 21 and S-metolachlor; compound 22 and S-metolachlor; compound 2 and sulfentrazone; compound 8 and sulfentrazone; compound 21 and sulfentrazone; compound 22 and sulfentrazone; compound 2 and thifensulfuron-methyl; compound 8 and thifensulfuron-methyl; compound 21 and thifensulfuron-methyl; compound 22 and thifensulfuron-methyl; compound 2 and triasulfuron; compound 8 and triasulfuron; compound 21 and triasulfuron; compound 22 and triasulfuron; compound 2 and tribenuron-methyl; compound 8 and tribenuron-methyl; compound 21 and tribenuron-methyl; compound 22 and tribenuron-methyl; compound 2 and triclopyr; compound 8 and triclopyr; compound 21 and triclopyr; compound 22 and triclopyr; compound 2 and triclopyr-butotyl; compound 8 and triclopyr-butotyl; compound 21 and triclopyr-butotyl; compound 22 and triclopyr-butotyl; compound 2 and triclopyr-triethylammonium; compound 8 and triclopyr-triethylammonium; compound 21 and triclopyr-triethylammonium; compound 22 and triclopyr-triethylammonium.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

Compounds of this invention can also be used in combination with herbicide safeners such as allidochlor, benoxacor, BCS (1-bromo-4-[(chloromethyl)sulfonyl]benzene), cloquintocet-mexyl, cyometrinil, cyprosulfonamide, dichlormid, 4-(dichloroacetyl)-1-oxa-4-azospiro[4.5]decane (MON 4660), 2-(dichloromethyl)-2-methyl-1,3-dioxolane (MG 191), dicyclonon, dietholate, fenchlorazole-ethyl, fenclorim, flurazole, fluxofenim, furilazole, isoxadifen-ethyl, mefenpyr-diethyl, mephenate, methoxyphenone ((4-methoxy-3-methylphenyl)(3-methylphenyl)methanone), naphthalic anhydride (1,8-naphthalic anhydride) and oxabetrinil to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

Combinations of compounds of the present invention with one or more other compounds or agents selected from herbicides and herbicide safeners are particularly useful for achieved desired spectra of weed control and safety to crops and other desired plants. Therefore of note is a herbicidal composition comprising a compound of Formula 1, or an N-oxide or a salt thereof, wherein the sum of n and m is 1 or 2 (i.e. in a herbicidally effective amount), at least one additional active ingredient (i.e. in an effective amount) selected from the group consisting of other herbicides and herbicide safeners, and at least one component selected from the group consisting of surfactants, solid diluents and liquid diluents.

The following Tests demonstrate the control efficacy of the compounds of this invention against specific weeds. The weed control afforded by the compounds is not limited, however, to these species. See Index Tables A-G for compound descriptions. The abbreviation “Ex.” stands for “Example” and is followed by a number indicating in which Synthesis Example the compound is prepared.

INDEX TABLE A

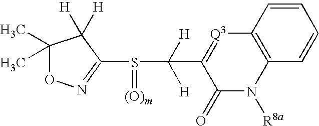

| Compound | Q³ | R^{8a} | m | m.p. (° C.) |
|---|---|---|---|---|
| 1 (Ex. 7) | C(CF₃) | CH₃ | 2 | ** |
| 12 (Ex. 6) | C(CF₃) | CH₃ | 0 | ** |
| 20 | N | CH₃ | 0 | 120-122 |
| 23 | N | CH₃ | 2 | 168-170 |
| 24 | N | CH₃ | 1 | 119-121 |
| 25 | N | CH₂CH₃ | 0 | 103-105 |
| 28 | N | CH₂CH₃ | 2 | 175-177 |
| 29 | N | CH₂CH₃ | 1 | 141-143 |

** See Synthesis Example for ¹H NMR data.

INDEX TABLE B

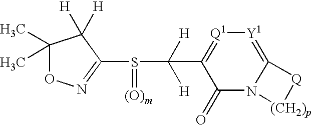

| Compound | Q¹ | Y¹ | Q | p | m | m.p. (° C.) |
|---|---|---|---|---|---|---|
| 2 (Ex. 9) | C(CF₃) | N | O | 2 | 2 | 197-198 |
| 3 | C(CF₃) | N | O | 3 | 1 | 161-162 |
| 4 | C(CF₃) | N | O | 3 | 2 | 190-191 |
| 5 | C(CF₃) | N | S | 2 | 1 | * |
| 6 | C(CF₃) | N | S | 2 | 2 | 185-186 |
| 13 (Ex. 8) | C(CF₃) | N | O | 2 | 0 | ** |
| 36 | C(CClF₂) | N | S | 3 | 1 | 142-144 |
| 39 | C(CClF₂) | N | S | 3 | 0 | 116-118 |
| 40 | C(CF₂CF₃) | N | S | 3 | 1 | *** |
| 41 | C(CF₂CF₃) | N | S | 2 | 1 | *** |
| 55 | C(CF₂CF₃) | N | O | 3 | 2 | 192-195 |

* See Index Table H for ¹H NMR data.
** See Synthesis Example for ¹H NMR data.
*** See Index Table I for mass spectra data.

INDEX TABLE C

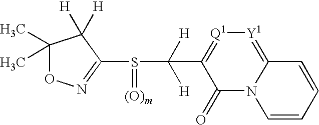

| Compound | Q¹ | Y¹ | m | m.p. (° C.) |
|---|---|---|---|---|
| 7 (Ex. 11) | C(CF₃) | N | 2 | 173-174 |
| 14 (Ex. 10) | C(CF₃) | N | 0 | ** |

** See Synthesis Example for ¹H NMR data.

INDEX TABLE D

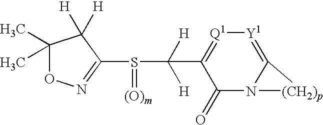

| Compound | Q¹ | Y¹ | p | m | m.p. (° C.) |
|---|---|---|---|---|---|
| 8 (Ex. 5) | C(CF₃) | N | 4 | 2 | 149-150 |
| 9 (Ex. 1) | C(CF₃) | N | 3 | 0 | 95-96 |
| 10 (Ex. 3) | C(CF₃) | N | 3 | 1 | 128-129 |
| 11 (Ex. 2) | C(CF₃) | N | 3 | 2 | 165-166 |
| 15 (Ex. 4) | C(CF₃) | N | 4 | 0 | ** |
| 17 | C(CClF₂) | N | 3 | 1 | 99-103 |
| 18 | C(CClF₂) | N | 3 | 2 | 190-193 |
| 19 | C(CF₂CF₃) | N | 4 | 1 | *** |
| 21 | C(CF₂CF₃) | N | 4 | 2 | 167-169 |
| 22 | C(CF₂CF₃) | N | 3 | 2 | 208-210 |
| 26 | C(CF₂CF₃) | N | 3 | 0 | 107-109 |
| 27 | C(CF₂CF₃) | N | 3 | 1 | *** |
| 30 | C(CClF₂) | N | 4 | 2 | 159-161 |
| 31 | C(CClF₂) | N | 4 | 0 | 109-111 |
| 32 | C(CClF₂) | N | 4 | 1 | *** |
| 33 | C(CF₃) | N | 5 | 2 | 142-144 |
| 34 | C(CF₃) | N | 5 | 1 | *** |
| 35 | C(CF₃) | N | 5 | 0 | 111-113 |

** See Synthesis Example for ¹H NMR data.
*** See Index Table I for mass spectra data.

INDEX TABLE E

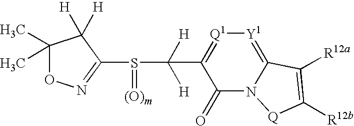

| Compound | R^{12a} | R^{12b} | Q¹ | Y¹ | Q | m | m.p. (° C.) |
|---|---|---|---|---|---|---|---|
| 16 | H | H | C(CF₃) | N | S | 2 | 179-180 |
| 42 | H | H | C(CF₃) | N | O | 2 | 203-206 |
| 43 | H | H | C(CF₃) | N | O | 1 | 165-166 |
| 44 | H | H | C(CF₃) | N | O | 0 | 111-112 |
| 47 | CH₃ | H | C(CF₃) | N | O | 1 | *** |
| 49 | CH₃ | H | C(CF₃) | N | O | 0 | 115-116 |
| 50 | H | CH₃ | C(CF₃) | N | O | 1 | 153-154 |
| 51 | H | CH₃ | C(CF₃) | N | O | 0 | 136-137 |
| 53 | H | CH₃ | C(CF₃) | N | O | 2 | 189-191 |
| 56 | CH₃ | H | C(CF₃) | N | O | 2 | 182-184 |

*** See Index Table I for mass spectra data.

INDEX TABLE F

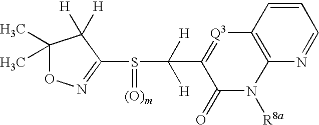

| Compound | Q³ | R^{8a} | m | m.p. (° C.) |
|---|---|---|---|---|
| 37 | N | CH₂CH₃ | 0 | 100-102 |
| 38 | N | CH₂CH₃ | 1 | 120-121 |
| 57 | N | CH₂CH₃ | 2 | 188-190 |

INDEX TABLE G

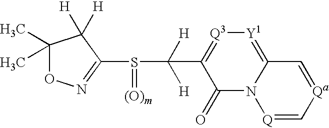

| Compound | Y¹ | Q | Q^{a} | Q³ | m | m.p. (° C.) |
|---|---|---|---|---|---|---|
| 45 | N | N | CH | C(CF₃) | 2 | 212-214 |
| 46 | N | N | CH | C(CF₃) | 1 | 154-155 |
| 48 | N | CH | N | C(CF₃) | 0 | 140-141 |
| 52 | N | CH | N | C(CF₃) | 2 | 113-115 |
| 54 | N | CH | N | C(CF₃) | 1 | 134-136 |

INDEX TABLE H

| Compound | ¹H NMR Data (CDCl₃ solution unless indicated otherwise)^{a} |
|---|---|
| 5 | δ 4.44 (m, 3H), 4.22 (m, 1H), 3.50 (m, 2H), 3.21 |
| | (m, 1H), 3.13 (m, 1H), 1.48 (s, 3H), 1.39 (s, 3H). |

^{a1}H NMR data are in ppm downfield from tetramethylsilane. Couplings are designated by (s)-singlet, (m)-multiplet.

INDEX TABLE I

| | Mass Specta Data (ESI MS, m/z |
|---|---|
| Compound | unless indicated otherwise)^{b} |
| 19 | 428 |
| 27 | 414 |
| 32 | 394 |
| 34 | 392 |
| 40 | 446 |
| 41 | 432 |
| 47 | 378 (AP⁺)^{c} |

^{b}The mass spectra (MS) value given is the molecular weight of the observed molecular ion formed by addition of H⁺ (molecular weight of 1) to the molecule having the greatest isotopic abundance (i.e. M), observed by mass spectrometry using electrospray ionization (ESI).
^{c}Mass spectra is reported as the molecular weight of the highest isotopic abundance parent ion (M + 1) formed by addition of H⁺ (molecular weight of 1) to the molecule, observed by mass spectrometry using atmospheric pressure chemical ionization (AP⁺)

### BIOLOGICAL EXAMPLES OF THE INVENTION

### Test A

Seeds of barnyardgrass (Echinochloa crus-galli), large crabgrass (Digitaria sanguinalis), giant foxtail (Setaria faberi), morningglory (Ipomoea spp.), pigweed (Amaranthus retroflexus), velvetleaf (Abutilon theophrasti), wheat (Triticum aestivum), and corn (Zea mays) were planted into a blend of loam soil and sand and treated preemergence with a directed soil spray using test chemicals formulated in a non-phytotoxic solvent mixture which included a surfactant. At the same time these species were also treated with postemergence applications of test compounds formulated in the same manner.

Plants ranged in height from two to ten cm and were in the one- to two-leaf stage for the postemergence treatment. Treated plants and untreated controls were maintained in a greenhouse for approximately ten days, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE A

| | Compound |
|---|---|
| 1000 g ai/ha | 9 |

Postemergence

| | |
|---|---|
| Barnyardgrass | 0 |
| Corn | 0 |
| Crabgrass, Large | 0 |
| Foxtail, Giant | 0 |
| Morningglory | 30 |
| Pigweed | 10 |
| Velvetleaf | 10 |
| Wheat | 0 |

| | Compounds | | | | | | | | | | | | | |
|---|---|---|---|---|---|---|---|---|---|---|---|---|---|---|
| 500 g ai/ha | 1 | 2 | 3 | 4 | 5 | 6 | 7 | 8 | 10 | 11 | 16 | 17 | 18 | 19 |

Postemergence

| Barnyardgrass | 80 | 80 | 50 | 80 | 60 | 80 | 80 | 90 | 80 | 90 | 90 | 60 | 80 | 70 |
|---|---|---|---|---|---|---|---|---|---|---|---|---|---|---|
| Corn | 0 | 50 | 0 | 50 | 30 | 0 | 0 | 60 | 20 | 50 | 0 | 0 | 40 | 40 |
| Crabgrass, Large | 70 | 90 | 50 | 80 | 70 | 90 | 80 | 90 | 70 | 90 | 90 | 50 | 90 | 30 |
| Foxtail, Giant | 80 | 80 | 50 | 80 | 70 | 90 | 80 | 90 | 70 | 80 | 90 | 60 | 80 | 50 |
| Morningglory | 0 | 70 | 0 | 50 | 0 | 50 | 0 | 90 | 20 | 60 | 50 | 0 | 60 | 20 |
| Pigweed | 50 | 60 | 30 | 60 | 10 | 90 | 0 | 60 | 30 | 70 | 50 | 10 | 40 | 10 |
| Velvetleaf | 20 | 60 | 50 | 70 | 40 | 80 | 20 | 70 | 40 | 80 | 60 | 10 | 60 | 20 |
| Wheat | 0 | 60 | 0 | 10 | 0 | 30 | 0 | 60 | 20 | 60 | 50 | 20 | 60 | 20 |
| | Compounds | | | | | | | | | | | | | |
| 500 g ai/ha | 21 | 22 | 23 | 24 | 27 | 28 | 29 | 30 | 32 | 33 | 34 | 36 | 38 | 40 |

Postemergence

| | | | | | | | | | | | | | | |
|---|---|---|---|---|---|---|---|---|---|---|---|---|---|---|
| Barnyardgrass | 90 | 80 | 60 | 0 | 70 | 40 | 20 | 80 | 40 | 80 | 50 | 50 | 30 | 70 |
| Corn | 0 | 0 | 0 | 0 | 0 | 0 | 0 | 0 | 0 | 0 | 0 | 0 | 0 | 20 |
| Crabgrass, Large | 80 | 80 | 60 | 0 | 80 | 50 | 0 | 80 | 40 | 80 | 40 | 50 | 20 | 40 |
| Foxtail, Giant | 70 | 80 | 60 | 0 | 70 | 0 | 0 | 80 | 50 | 80 | 60 | 60 | 0 | 50 |
| Morningglory | 40 | 50 | 0 | 0 | 40 | 0 | 0 | 50 | 50 | 60 | 40 | 30 | 10 | 10 |
| Pigweed | 10 | 40 | 0 | 0 | 30 | 0 | 0 | 60 | 30 | 50 | 0 | 0 | 0 | 0 |
| Velvetleaf | 10 | 60 | 0 | 0 | 40 | 0 | 0 | 50 | 10 | 20 | 0 | 0 | 30 | 0 |
| Wheat | 20 | 50 | 0 | 0 | 50 | 0 | 0 | 50 | 0 | 40 | 20 | 20 | 10 | 50 |

| | Compounds | | | | | | | | | | | | |
|---|---|---|---|---|---|---|---|---|---|---|---|---|---|
| 500 g ai/ha | 41 | 42 | 43 | 45 | 46 | 47 | 50 | 52 | 53 | 54 | 55 | 56 | 57 |

Postemergence

| | | | | | | | | | | | | | |
|---|---|---|---|---|---|---|---|---|---|---|---|---|---|
| Barnyardgrass | 70 | 80 | 70 | 70 | 0 | 50 | 60 | 90 | 90 | 0 | 90 | 80 | 30 |
| Corn | 40 | 0 | 40 | 0 | 0 | 20 | 30 | 0 | 0 | 0 | 0 | 0 | 0 |
| Crabgrass, Large | 50 | 70 | 60 | 70 | 0 | 60 | 50 | 50 | 80 | 20 | 70 | 80 | 40 |
| Foxtail, Giant | 60 | 80 | 70 | 60 | 0 | 60 | 60 | 80 | 80 | 50 | 80 | 80 | 30 |
| Morningglory | 10 | 0 | 40 | 30 | 0 | 20 | 10 | 30 | 10 | 10 | 20 | 30 | 0 |
| Pigweed | 0 | 10 | 0 | 10 | 0 | 0 | 0 | 0 | 0 | 0 | 10 | 10 | 0 |
| Velvetleaf | 0 | 0 | 0 | 0 | 0 | 0 | 0 | 0 | 0 | 0 | 30 | 0 | 0 |
| Wheat | 40 | 20 | 0 | 0 | 0 | 0 | 0 | 30 | 20 | 0 | 30 | 0 | 0 |

| | Compounds | | | | | | | | | | | | | |
|---|---|---|---|---|---|---|---|---|---|---|---|---|---|---|
| 125 g ai/ha | 1 | 2 | 3 | 4 | 5 | 6 | 7 | 8 | 10 | 11 | 16 | 17 | 18 | 19 |

Postemergence

| Barnyardgrass | 0 | 70 | 0 | 70 | 30 | 70 | 80 | 80 | 30 | 80 | 80 | 0 | 80 | 10 |
|---|---|---|---|---|---|---|---|---|---|---|---|---|---|---|
| Corn | 0 | 0 | 0 | 0 | 0 | 0 | 0 | 0 | 0 | 20 | 0 | 0 | 0 | 0 |
| Crabgrass, Large | 10 | 60 | 20 | 60 | 10 | 80 | 40 | 60 | 40 | 80 | 80 | 20 | 60 | 0 |
| Foxtail, Giant | 20 | 50 | 0 | 70 | 40 | 80 | 60 | 70 | 30 | 80 | 80 | 30 | 70 | 0 |
| Morningglory | 0 | 30 | 0 | 0 | 0 | 40 | 0 | 60 | 10 | 10 | 0 | 0 | 30 | 0 |
| Pigweed | 0 | 20 | 0 | 30 | 0 | 50 | 0 | 10 | 10 | 30 | 10 | 0 | 30 | 0 |
| Velvetleaf | 0 | 30 | 20 | 50 | 0 | 60 | 0 | 50 | 10 | 50 | 30 | 0 | 30 | 0 |
| Wheat | 0 | 0 | 0 | 0 | 0 | 20 | 0 | 20 | 0 | 20 | 0 | 0 | 0 | 0 |
| | Compounds | | | | | | | | | | | | | |
| 125 g ai/ha | 21 | 22 | 23 | 24 | 27 | 28 | 29 | 30 | 32 | 33 | 34 | 36 | 38 | 40 |

Postemergence

| | | | | | | | | | | | | | | |
|---|---|---|---|---|---|---|---|---|---|---|---|---|---|---|
| Barnyardgrass | 70 | 70 | 40 | 0 | 40 | 30 | 10 | 60 | 10 | 50 | 0 | 20 | 20 | 10 |
| Corn | 0 | 0 | 0 | 0 | 0 | 0 | 0 | 0 | 0 | 0 | 0 | — | 0 | 0 |
| Crabgrass, Large | 50 | 70 | 30 | 0 | 40 | 0 | 0 | 60 | 10 | 50 | 0 | 30 | 0 | 0 |
| Foxtail, Giant | 60 | 70 | 10 | 0 | 40 | 0 | 0 | 60 | 0 | 70 | 0 | 50 | 0 | 0 |
| Morningglory | 0 | 0 | 0 | 0 | 0 | 0 | 0 | 10 | 40 | 40 | 30 | 0 | 0 | 0 |
| Pigweed | 0 | 0 | 0 | 0 | 0 | 0 | 0 | 50 | 0 | 20 | 0 | 0 | 0 | 0 |
| Velvetleaf | 0 | 20 | 0 | 0 | 0 | 0 | 0 | 20 | 0 | 0 | 0 | 0 | 0 | 0 |
| Wheat | 0 | 20 | 0 | 0 | 0 | 0 | 0 | 0 | 0 | 0 | 0 | 0 | 0 | 0 |

| | Compounds | | | | | | | | | | | | |
|---|---|---|---|---|---|---|---|---|---|---|---|---|---|
| 125 g ai/ha | 41 | 42 | 43 | 45 | 46 | 47 | 50 | 52 | 53 | 54 | 55 | 56 | 57 |

Postemergence

| | | | | | | | | | | | | | |
|---|---|---|---|---|---|---|---|---|---|---|---|---|---|
| Barnyardgrass | 30 | 70 | 40 | 30 | 0 | 0 | 0 | 30 | 50 | 0 | 70 | 70 | 0 |
| Corn | 0 | 0 | 0 | 0 | 0 | 0 | 0 | 0 | 0 | 0 | 0 | 0 | 0 |
| Crabgrass, Large | 0 | 50 | 10 | 20 | 0 | 0 | 0 | 20 | 30 | 0 | 30 | 60 | 10 |
| Foxtail, Giant | 10 | 60 | 30 | 20 | 0 | 0 | 0 | 60 | 60 | 0 | 70 | 40 | 0 |
| Morningglory | 0 | 0 | 0 | 0 | 0 | 0 | 0 | 0 | 0 | 0 | 10 | 10 | 0 |
| Pigweed | 0 | 0 | 0 | 0 | 0 | 0 | 0 | 0 | 0 | 0 | 0 | 0 | 0 |
| Velvetleaf | 0 | 0 | 0 | 0 | 0 | 0 | 0 | 0 | 0 | 0 | 0 | 0 | 0 |
| Wheat | 0 | 0 | 0 | 0 | 0 | 0 | 0 | 0 | 0 | 0 | 0 | 0 | 0 |

| | |
|---|---|
| | Compound |
| 1000 g ai/ha | 9 |

Preemergence

| | |
|---|---|
| Barnyardgrass | 0 |
| Corn | 0 |
| Crabgrass, Large | 0 |
| Foxtail, Giant | 0 |
| Morningglory | 0 |
| Pigweed | 0 |
| Velvetleaf | 0 |
| Wheat | 0 |

| | Compounds | | | | | | | | | | | | | |
|---|---|---|---|---|---|---|---|---|---|---|---|---|---|---|
| 500 g ai/ha | 1 | 2 | 3 | 4 | 5 | 6 | 7 | 8 | 10 | 11 | 16 | 17 | 18 | 19 |

Preemergence

| Barnyardgrass | 80 | 90 | 60 | 100 | 70 | 90 | 90 | 90 | 70 | 90 | 80 | 60 | 100 | 80 |
|---|---|---|---|---|---|---|---|---|---|---|---|---|---|---|
| Corn | — | 50 | 0 | 50 | 0 | 0 | 0 | 20 | 10 | 0 | 0 | 0 | 0 | 20 |
| Crabgrass, Large | 50 | 100 | 70 | 100 | 80 | 100 | 80 | 100 | 80 | 90 | 90 | 80 | 100 | 80 |
| Foxtail, Giant | 90 | 100 | 70 | 100 | 80 | 100 | 90 | 100 | 80 | 100 | 90 | 80 | 100 | 80 |
| Morningglory | 0 | 80 | 0 | 70 | 0 | 100 | 0 | 90 | 0 | 90 | 80 | 0 | 50 | 60 |
| Pigweed | 60 | 60 | 60 | 100 | 60 | 100 | 0 | 100 | 0 | 100 | 100 | 0 | 100 | 20 |
| Velvetleaf | 0 | 90 | 0 | 90 | 0 | 80 | 0 | 90 | 0 | 60 | 60 | 0 | 60 | 0 |
| Wheat | 0 | 60 | 0 | 40 | 0 | 0 | 10 | 60 | 30 | 60 | 0 | 0 | 30 | 20 |
| | Compounds | | | | | | | | | | | | | |
| 500 g ai/ha | 21 | 22 | 23 | 24 | 27 | 28 | 29 | 30 | 32 | 33 | 34 | 36 | 38 | 40 |

Preemergence

| | | | | | | | | | | | | | | |
|---|---|---|---|---|---|---|---|---|---|---|---|---|---|---|
| Barnyardgrass | 100 | 90 | 40 | 0 | 90 | 10 | 0 | 100 | 70 | 90 | 70 | 60 | 0 | 60 |
| Corn | 0 | 0 | 0 | 0 | 0 | 0 | — | 0 | 0 | 0 | 0 | 10 | 0 | 0 |
| Crabgrass, Large | 100 | 100 | 70 | 0 | 90 | 50 | 0 | 100 | 80 | 100 | 80 | 50 | 0 | 70 |
| Foxtail, Giant | 100 | 100 | 80 | 0 | 90 | 20 | 0 | 100 | 80 | 100 | 90 | 70 | 0 | 80 |
| Morningglory | 50 | 80 | — | 0 | 0 | 0 | 0 | 40 | 0 | 50 | 0 | 0 | 0 | 0 |
| Pigweed | 100 | 90 | 10 | 0 | 0 | 0 | 0 | 100 | 0 | 100 | 0 | 0 | 0 | 0 |
| Velvetleaf | 60 | 90 | 0 | 0 | 60 | 0 | 0 | 60 | 0 | 60 | 0 | 0 | 0 | 0 |
| Wheat | 40 | 50 | 0 | 0 | 50 | 0 | 0 | 50 | 0 | 50 | 50 | 0 | 0 | 0 |

| | Compounds | | | | | | | | | | | | |
|---|---|---|---|---|---|---|---|---|---|---|---|---|---|
| 500 g ai/ha | 41 | 42 | 43 | 45 | 46 | 47 | 50 | 52 | 53 | 54 | 55 | 56 | 57 |

Preemergence

| | | | | | | | | | | | | | |
|---|---|---|---|---|---|---|---|---|---|---|---|---|---|
| Barnyardgrass | 60 | 80 | 70 | 90 | 0 | 70 | 70 | 80 | 90 | 0 | 80 | 90 | 40 |
| Corn | 0 | 0 | 0 | 0 | 0 | 0 | 0 | 0 | 0 | 0 | 0 | 0 | 0 |
| Crabgrass, Large | 80 | 80 | 60 | 90 | 0 | 20 | 70 | 80 | 90 | 0 | 90 | 90 | 50 |
| Foxtail, Giant | 80 | 90 | 80 | 90 | 0 | 30 | 70 | 90 | 90 | 0 | 90 | 90 | 40 |
| Morningglory | 0 | 0 | 0 | 0 | 0 | 0 | 0 | 0 | — | 0 | 50 | 0 | 0 |
| Pigweed | 0 | 30 | 0 | 0 | 0 | 0 | 0 | 0 | 0 | 0 | 40 | 0 | 0 |
| Velvetleaf | 0 | 0 | 0 | 0 | 20 | 0 | 0 | 0 | 0 | 0 | 30 | 0 | 0 |
| Wheat | 0 | 0 | 0 | 10 | 0 | 0 | 0 | 0 | 0 | 0 | 20 | 0 | 0 |

| | Compounds | | | | | | | | | | | | | |
|---|---|---|---|---|---|---|---|---|---|---|---|---|---|---|
| 125 g ai/ha | 1 | 2 | 3 | 4 | 5 | 6 | 7 | 8 | 10 | 11 | 16 | 17 | 18 | 19 |

Preemergence

| Barnyardgrass | 0 | 80 | 30 | 70 | 0 | 70 | 60 | 90 | 0 | 60 | 50 | 20 | 70 | 0 |
|---|---|---|---|---|---|---|---|---|---|---|---|---|---|---|
| Corn | 0 | 0 | 0 | 0 | 0 | 0 | 0 | 0 | 0 | 0 | 0 | 0 | 0 | — |
| Crabgrass, Large | 0 | 90 | 50 | 60 | 40 | 80 | 60 | 90 | 0 | 80 | 90 | 40 | 90 | 0 |
| Foxtail, Giant | 30 | 90 | 40 | 90 | 30 | 80 | 80 | 100 | 20 | 90 | 90 | 30 | 90 | 0 |
| Morningglory | 0 | 0 | 0 | 0 | 0 | 90 | 0 | 20 | 0 | 30 | 70 | 0 | 0 | 0 |
| Pigweed | 0 | 20 | 40 | 30 | 40 | 60 | 0 | 30 | 0 | 30 | — | 0 | 60 | 0 |
| Velvetleaf | 0 | 10 | 0 | 10 | 0 | 0 | 0 | 20 | 0 | 10 | 50 | 0 | 0 | 0 |
| Wheat | 0 | 10 | 0 | 0 | 0 | 0 | 0 | 0 | 0 | 0 | 0 | 0 | 0 | 0 |
| | Compounds | | | | | | | | | | | | | |
| 125 g ai/ha | 21 | 22 | 23 | 24 | 27 | 28 | 29 | 30 | 32 | 33 | 34 | 36 | 38 | 40 |

Preemergence

| | | | | | | | | | | | | | | |
|---|---|---|---|---|---|---|---|---|---|---|---|---|---|---|
| Barnyardgrass | 90 | 80 | 20 | 0 | 50 | 0 | 0 | 50 | 20 | 40 | 0 | 0 | 0 | 0 |
| Corn | 0 | 0 | 0 | 0 | 0 | 0 | 0 | 0 | 0 | 0 | 0 | 0 | 0 | 0 |
| Crabgrass, Large | 90 | 90 | 10 | 0 | 70 | 40 | 0 | 90 | 50 | 90 | 0 | 30 | 0 | 0 |
| Foxtail, Giant | 90 | 90 | 30 | 0 | 60 | 0 | 0 | 90 | 30 | 90 | 0 | 30 | 0 | 0 |
| Morningglory | 0 | 50 | 0 | 0 | 0 | 0 | 0 | 30 | 0 | 20 | 0 | 0 | 0 | 0 |
| Pigweed | 0 | 10 | 0 | 0 | 0 | 0 | 0 | 70 | 0 | 10 | 0 | 0 | 0 | 0 |
| Velvetleaf | 0 | 30 | 0 | 0 | 0 | 0 | 0 | 40 | 0 | 30 | 0 | 0 | 0 | 0 |
| Wheat | 0 | 0 | 0 | 0 | 0 | 0 | 0 | 0 | 0 | 0 | 0 | 0 | 0 | 0 |

| | Compounds | | | | | | | | | | | | |
|---|---|---|---|---|---|---|---|---|---|---|---|---|---|
| 125 g ai/ha | 41 | 42 | 43 | 45 | 46 | 47 | 50 | 52 | 53 | 54 | 55 | 56 | 57 |

Preemergence

| | | | | | | | | | | | | | |
|---|---|---|---|---|---|---|---|---|---|---|---|---|---|
| Barnyardgrass | 0 | 30 | 10 | 20 | 0 | 0 | 0 | 20 | 30 | 0 | 50 | 0 | 0 |
| Corn | 0 | 0 | 0 | 0 | 0 | 0 | 0 | 0 | 0 | 0 | 0 | 0 | 0 |
| Crabgrass, Large | 0 | 50 | 0 | 20 | 0 | 0 | 0 | 20 | 30 | 0 | 0 | 0 | 40 |
| Foxtail, Giant | 0 | 60 | 0 | 30 | 0 | 0 | 0 | 40 | 30 | 0 | 80 | 20 | 0 |
| Morningglory | — | 0 | 0 | 0 | 0 | 0 | 0 | 0 | 0 | 0 | 0 | 0 | 0 |
| Pigweed | 0 | 0 | 0 | — | 0 | 0 | 0 | 0 | 0 | 0 | 0 | 0 | 0 |
| Velvetleaf | 0 | 0 | 0 | 0 | 0 | 0 | 0 | 0 | 0 | 0 | 0 | 0 | 0 |
| Wheat | 0 | 0 | 0 | 0 | 0 | 0 | 0 | 0 | 0 | 0 | 0 | 0 | 0 |

### Test B

Seeds of plant species selected from blackgrass (Alopecurus myosuroides), downy bromegrass (Bromus tectorum), green foxtail (Setaria viridis), Italian ryegrass (Lolium multiflorum), wheat (Triticum aestivum), wild oat (Avena fatua), catchweed bedstraw (Galium aparine), bermudagrass (Cynodon dactylon), Surinam grass (Brachiaria decumbens), cocklebur (Xanthium strumarium), corn (Zea mays), large crabgrass (Digitaria sanguinalis), woolly cupgrass (Eriochloa villosa), giant foxtail (Setaria faberii), goosegrass (Eleusine indica), johnsongrass (Sorghum halepense), kochia (Kochia scoparia), lambsquarters (Chenopodium album), morningglory (Ipomoea coccinea), nightshade (eastern black nightshade, Solanum ptycanthum), yellow nutsedge (Cyperus esculentus), pigweed (Amaranthus retroflexus), ragweed (common ragweed, Ambrosia elatior), soybean (Glycine max), sunflower (common oilseed sunflower, Helianthus annuus), Russian thistle (Salsola kali), and velvetleaf (Abutilon theophrasti) were planted into a blend of loam soil and sand and treated preemergence with test compounds formulated in a non-phytotoxic solvent mixture which included a surfactant.

Plant species in the flooded paddy test consisted of rice (Oryza sativa), umbrella sedge (Cyperus difformis), ducksalad (Heteranthera limosa) and barnyardgrass (Echinochloa crus-galli) grown to the 2-leaf stage for testing. At time of treatment, test pots were flooded to 3 cm above the soil surface, treated by application of test compounds directly to the paddy water, and then maintained at that water depth for the duration of the test.

Treated plants and controls were maintained in a greenhouse for 13 to 15 days, after which time all species were compared to controls and visually evaluated. Plant response ratings, summarized in Table B, are based on a scale of 0 to 100 where 0 is no effect and 100 is complete control. A dash (-) response means no test result.

TABLE B

| | Compounds | | | | | | | | | | | | | |
|---|---|---|---|---|---|---|---|---|---|---|---|---|---|---|
| 250 g ai/ha | 1 | 2 | 3 | 4 | 5 | 6 | 7 | 8 | 10 | 11 | 16 | 17 | 18 | 19 |

Flood

| Barnyardgrass | 20 | 0 | 0 | 0 | 30 | 0 | 0 | 0 | 0 | 20 | 0 | 0 | 0 | 0 |
|---|---|---|---|---|---|---|---|---|---|---|---|---|---|---|
| Ducksalad | 0 | 0 | 40 | 0 | 20 | 65 | 0 | 20 | 0 | 35 | 40 | 0 | 0 | 0 |
| Rice | 20 | 0 | 0 | 0 | 0 | 20 | 0 | 0 | 0 | 25 | 0 | 0 | 0 | 0 |
| Sedge, Umbrella | 0 | 0 | 0 | 0 | 45 | 75 | 0 | 20 | 0 | 20 | 40 | 0 | 0 | 0 |
| | Compounds | | | | | | | | | | | | | |
| 250 g ai/ha | 21 | 22 | 23 | 24 | 27 | 28 | 29 | 33 | 34 | 36 | 38 | 40 | 41 | 42 |

Flood

| | | | | | | | | | | | | | | |
|---|---|---|---|---|---|---|---|---|---|---|---|---|---|---|
| Barnyardgrass | 0 | 0 | 0 | 0 | 0 | 0 | 0 | 35 | 0 | 0 | 0 | 0 | 0 | 0 |
| Ducksalad | 0 | 0 | 0 | 30 | 0 | 0 | 20 | 50 | 0 | 0 | 0 | 0 | 0 | 0 |
| Rice | 0 | 10 | 0 | 30 | 0 | 0 | 0 | 25 | 0 | 0 | 0 | 0 | 0 | 0 |
| Sedge, Umbrella | 0 | 0 | 0 | 30 | 0 | 0 | 0 | 80 | 0 | 0 | 0 | 0 | 0 | 0 |

| | Compounds | | | | | | | | | | |
|---|---|---|---|---|---|---|---|---|---|---|---|
| 250 g ai/ha | 43 | 45 | 46 | 47 | 50 | 52 | 53 | 54 | 55 | 56 | 57 |

Flood

| | | | | | | | | | | | |
|---|---|---|---|---|---|---|---|---|---|---|---|
| Barnyardgrass | 0 | 0 | 0 | 0 | 0 | 0 | 0 | 0 | 0 | 0 | 0 |
| Ducksalad | 25 | 0 | 0 | 0 | 0 | 0 | 0 | 0 | 0 | 0 | 0 |
| Rice | 0 | 0 | 0 | 0 | 0 | 0 | 0 | 0 | 0 | 0 | 0 |
| Sedge, Umbrella | 30 | 0 | 0 | 0 | 0 | 0 | 0 | 0 | 0 | 30 | 0 |

| | Compounds | | | | | | | | | | | | | |
|---|---|---|---|---|---|---|---|---|---|---|---|---|---|---|
| 125 g ai/ha | 1 | 4 | 10 | 11 | 17 | 18 | 19 | 27 | 28 | 29 | 30 | 32 | 33 | 34 |

Flood

| | | | | | | | | | | | | | | |
|---|---|---|---|---|---|---|---|---|---|---|---|---|---|---|
| Barnyardgrass | 20 | 0 | 0 | 0 | 0 | 0 | 0 | 0 | 0 | 0 | 0 | 0 | 0 | 0 |
| Ducksalad | 0 | 0 | 0 | 0 | 0 | 0 | 0 | 0 | 0 | 0 | 0 | 0 | 20 | 0 |
| Rice | 0 | 0 | 0 | 25 | 0 | 0 | 0 | 0 | 0 | 0 | 0 | 0 | 0 | 0 |
| Sedge, Umbrella | 0 | 0 | 0 | 0 | 0 | 0 | 0 | 0 | 0 | 0 | 0 | 0 | 60 | 0 |

| | Compounds | | | | | | | | | | |
|---|---|---|---|---|---|---|---|---|---|---|---|
| 125 g ai/ha | 36 | 38 | 40 | 41 | 42 | 43 | 45 | 46 | 55 | 56 | 57 |

Flood

| | | | | | | | | | | | |
|---|---|---|---|---|---|---|---|---|---|---|---|
| Barnyardgrass | 0 | 0 | 0 | 0 | 0 | 0 | 0 | 0 | 0 | 0 | 0 |
| Ducksalad | 0 | 0 | 0 | 0 | 0 | 0 | 0 | 0 | 0 | 0 | 0 |
| Rice | 0 | 0 | 0 | 0 | 0 | 0 | 0 | 0 | 0 | 0 | 0 |
| Sedge, Umbrella | 0 | 0 | 0 | 0 | 0 | 20 | 0 | 0 | 0 | 20 | 0 |

| | Compounds | | | | | | | | | | | | | |
|---|---|---|---|---|---|---|---|---|---|---|---|---|---|---|
| 62 g ai/ha | 1 | 2 | 3 | 4 | 5 | 6 | 7 | 8 | 10 | 11 | 16 | 17 | 18 | 19 |

Flood

| Barnyardgrass | 20 | 0 | 0 | 0 | 10 | 0 | 0 | 0 | 0 | 0 | 0 | 0 | 0 | 0 |
|---|---|---|---|---|---|---|---|---|---|---|---|---|---|---|
| Ducksalad | 0 | 0 | 0 | 0 | 0 | 40 | 0 | 0 | 0 | 0 | 40 | 0 | 0 | 0 |
| Rice | 0 | 0 | 0 | 0 | 0 | 20 | 0 | 0 | 0 | 0 | 0 | 0 | 0 | 0 |
| Sedge, Umbrella | 0 | 0 | 0 | 0 | 0 | 0 | 0 | 0 | 0 | 0 | 0 | 0 | 0 | 0 |
| | Compounds | | | | | | | | | | | | | |
| 62 g ai/ha | 21 | 22 | 23 | 24 | 27 | 28 | 29 | 33 | 34 | 36 | 38 | 40 | 41 | 42 |

Flood

| | | | | | | | | | | | | | | |
|---|---|---|---|---|---|---|---|---|---|---|---|---|---|---|
| Barnyardgrass | 0 | 0 | 0 | 0 | 0 | 0 | 0 | 0 | 0 | 0 | 0 | 0 | 0 | 0 |
| Ducksalad | 0 | 0 | 0 | 0 | 0 | 0 | 0 | 0 | 0 | 0 | 0 | 0 | 0 | 0 |
| Rice | 0 | 0 | 0 | 30 | 0 | 0 | 0 | 0 | 0 | 0 | 0 | 0 | 0 | 0 |
| Sedge, Umbrella | 0 | 0 | 0 | 20 | 0 | 0 | 0 | 20 | 0 | 0 | 0 | 0 | 0 | 0 |

| | Compounds | | | | | | | | | | |
|---|---|---|---|---|---|---|---|---|---|---|---|
| 62 g ai/ha | 43 | 45 | 46 | 47 | 50 | 52 | 53 | 54 | 55 | 56 | 57 |

Flood

| | | | | | | | | | | | |
|---|---|---|---|---|---|---|---|---|---|---|---|
| Barnyardgrass | 0 | 0 | 0 | 0 | 0 | 0 | 0 | 0 | 0 | 0 | 0 |
| Ducksalad | 0 | 0 | 0 | 0 | 0 | 0 | 0 | 0 | 0 | 0 | 0 |
| Rice | 0 | 0 | 0 | 0 | 0 | 0 | 0 | 0 | 0 | 0 | 0 |
| Sedge, Umbrella | 0 | 0 | 0 | 0 | 0 | 0 | 0 | 0 | 0 | 0 | 0 |

| | Compounds | | | | | | | | | | | | | |
|---|---|---|---|---|---|---|---|---|---|---|---|---|---|---|
| 31 g ai/ha | 1 | 4 | 10 | 11 | 17 | 18 | 19 | 27 | 28 | 29 | 30 | 32 | 33 | 34 |

Flood

| | | | | | | | | | | | | | | |
|---|---|---|---|---|---|---|---|---|---|---|---|---|---|---|
| Barnyardgrass | 20 | 0 | 0 | 0 | 0 | 0 | 0 | 0 | 0 | 0 | 0 | 0 | 0 | 0 |
| Ducksalad | 0 | 0 | 0 | 0 | 0 | 0 | 0 | 0 | 0 | 0 | 0 | 0 | 0 | 0 |
| Rice | 0 | 0 | 0 | 0 | 0 | 0 | 0 | 0 | 0 | 0 | 0 | 0 | 0 | 0 |
| Sedge, Umbrella | 0 | 0 | 0 | 0 | 0 | 0 | 0 | 0 | 0 | 0 | 0 | 0 | 0 | 0 |

| | Compounds | | | | | | | | | | |
|---|---|---|---|---|---|---|---|---|---|---|---|
| 31 g ai/ha | 36 | 38 | 40 | 41 | 42 | 43 | 45 | 46 | 55 | 56 | 57 |

Flood

| | | | | | | | | | | | |
|---|---|---|---|---|---|---|---|---|---|---|---|
| Barnyardgrass | 0 | 0 | 0 | 0 | 0 | 0 | 0 | 0 | 0 | 0 | 0 |
| Ducksalad | 0 | 0 | 0 | 0 | 0 | 0 | 0 | 0 | 0 | 0 | 0 |
| Rice | 0 | 0 | 0 | 0 | 0 | 0 | 0 | 0 | 0 | 0 | 0 |
| Sedge, Umbrella | 0 | 0 | 0 | 0 | 0 | 0 | 0 | 0 | 0 | 0 | 0 |

| | Compounds | | | | | | |
|---|---|---|---|---|---|---|---|
| 125 g ai/ha | 6 | 8 | 11 | 16 | 18 | 21 | 22 |

Preemergence

| | | | | | | | |
|---|---|---|---|---|---|---|---|
| Bermudagrass | 100 | 100 | 100 | — | 100 | 100 | 100 |
| Blackgrass | 95 | 100 | 100 | 100 | 98 | 98 | 98 |
| Bromegrass, Downy | 50 | 40 | 65 | 30 | 20 | 60 | 60 |
| Cocklebur | 0 | 0 | 0 | 0 | 0 | 0 | 0 |
| Corn | 0 | 55 | 0 | 0 | 0 | 10 | 10 |
| Crabgrass, Large | 100 | 100 | 100 | 100 | 100 | 100 | 100 |
| Cupgrass, Woolly | 100 | 100 | 100 | 98 | 95 | 100 | 100 |
| Foxtail, Giant | 100 | 100 | 100 | 98 | 98 | 85 | 100 |
| Foxtail, Green | 100 | 98 | 100 | 98 | 98 | 100 | 100 |
| Galium | 100 | 100 | 98 | 70 | 0 | 50 | 0 |
| Goosegrass | 100 | 100 | 100 | 100 | 98 | 100 | 100 |
| Johnsongrass | 100 | 100 | 90 | 95 | 85 | 98 | 98 |
| Kochia | — | — | — | 0 | 100 | 80 | 70 |
| Lambsquarters | 95 | 100 | 100 | 98 | 85 | 98 | 100 |
| Morningglory | 0 | 80 | 95 | 0 | 45 | 100 | 100 |
| Nightshade | 98 | 98 | 98 | 95 | 95 | 90 | 100 |
| Nutsedge, Yellow | 45 | 80 | 0 | 0 | 45 | 90 | 80 |
| Oat, Wild | 60 | 90 | 65 | 60 | 45 | 40 | 40 |
| Pigweed | 100 | 100 | 100 | 100 | 100 | 98 | 100 |
| Ragweed | 35 | 60 | 55 | 0 | 0 | 10 | 50 |
| Russian Thistle | — | 0 | — | — | — | 85 | 60 |
| Ryegrass, Italian | 95 | 100 | 90 | 60 | 40 | 40 | 100 |
| Soybean | 0 | 0 | 0 | 0 | 0 | 0 | 0 |
| Sunflower | 0 | 45 | 20 | 0 | 0 | 10 | 50 |
| Surinam Grass | 100 | 100 | 85 | 98 | 85 | 100 | 95 |
| Velvetleaf | 0 | 45 | 65 | 0 | 0 | 75 | 100 |
| Wheat | 0 | 40 | 50 | 0 | — | 0 | 10 |

| | Compounds | | | | | | | |
|---|---|---|---|---|---|---|---|---|
| 62 g ai/ha | 2 | 6 | 8 | 11 | 16 | 18 | 21 | 22 |

Preemergence

| Bermudagrass | 85 | 100 | 100 | 100 | — | 100 | 100 | 100 |
|---|---|---|---|---|---|---|---|---|
| Blackgrass | 50 | 95 | 90 | 70 | 90 | 10 | 50 | 40 |
| Bromegrass, Downy | 0 | 20 | 20 | 0 | 0 | 0 | 0 | 0 |
| Cocklebur | 0 | 0 | 0 | 0 | 0 | 0 | 0 | 0 |
| Corn | 20 | 0 | 45 | 0 | 0 | 0 | 0 | 0 |
| Crabgrass, Large | 90 | 100 | 100 | 100 | 100 | 90 | 98 | 98 |
| Cupgrass, Woolly | 95 | 98 | 95 | 100 | 95 | 65 | 100 | 100 |
| Foxtail, Giant | 98 | 100 | 98 | 95 | 90 | 85 | 80 | 90 |
| Foxtail, Green | 85 | 50 | 98 | 100 | 85 | 98 | 100 | 100 |
| Galium | 100 | 50 | 100 | 95 | 0 | 0 | 30 | 0 |
| Goosegrass | 80 | 100 | 100 | 100 | 100 | 98 | 100 | 100 |
| Johnsongrass | 80 | 98 | 100 | 65 | 85 | 60 | 60 | 65 |
| Kochia | 70 | — | — | — | 0 | 55 | 60 | 0 |
| Lambsquarters | 0 | 90 | 100 | 0 | 0 | 75 | 0 | 70 |
| Morningglory | 0 | 0 | 20 | 80 | 0 | 0 | 80 | 70 |
| Nightshade | 0 | 95 | 75 | 95 | 85 | 0 | 75 | 90 |
| Nutsedge, Yellow | 0 | 0 | 60 | 0 | 0 | 20 | 60 | 60 |
| Oat, Wild | 25 | 60 | 35 | 20 | 30 | 0 | 30 | 30 |
| Pigweed | 0 | 100 | 95 | 100 | 0 | 100 | 0 | — |
| Ragweed | 20 | 25 | 25 | 50 | 0 | 0 | 0 | 30 |
| Russian Thistle | 0 | — | 0 | — | — | — | 65 | 30 |
| Ryegrass, Italian | 0 | 80 | 35 | 0 | 30 | 35 | 0 | 100 |
| Soybean | 0 | 0 | 0 | 0 | 0 | 0 | 0 | 0 |
| Sunflower | 0 | 0 | 40 | 0 | 0 | 0 | 0 | 0 |
| Surinam Grass | 80 | 98 | 100 | 0 | 95 | 75 | 50 | 90 |
| Velvetleaf | 0 | 0 | 35 | 0 | 0 | 0 | 0 | 60 |
| Wheat | 0 | 0 | 0 | 20 | 0 | 50 | 0 | 0 |
| | Compounds | | | | | | | |
| 31 g ai/ha | 2 | 6 | 8 | 11 | 16 | 18 | 21 | 22 |

Preemergence

| Bermudagrass | 75 | 100 | 100 | 100 | — | 98 | 100 | 100 |
|---|---|---|---|---|---|---|---|---|
| Blackgrass | 0 | 80 | 90 | 70 | 85 | 0 | 40 | 40 |
| Bromegrass, Downy | 0 | 20 | 0 | 0 | 0 | 0 | 0 | 0 |
| Cocklebur | 0 | 0 | 0 | 0 | 0 | 0 | 0 | 0 |
| Corn | 0 | 0 | 15 | 0 | 0 | 0 | 0 | 0 |
| Crabgrass, Large | 80 | 100 | 95 | 85 | 95 | 70 | 95 | 90 |
| Cupgrass, Woolly | 75 | 98 | 95 | 100 | 25 | 20 | 100 | 100 |
| Foxtail, Giant | 70 | 95 | 90 | 40 | 80 | 80 | 60 | 50 |
| Foxtail, Green | 55 | 50 | 85 | 70 | 20 | 80 | 45 | 70 |
| Galium | 100 | 50 | 0 | 0 | 0 | — | 0 | 0 |
| Goosegrass | 75 | 100 | 98 | 90 | 95 | 95 | 98 | 80 |
| Johnsongrass | 30 | 70 | 90 | 60 | 45 | 20 | 30 | 10 |
| Kochia | 75 | — | — | — | 0 | 0 | — | 0 |
| Lambsquarters | 0 | 20 | 98 | 0 | 0 | 0 | 0 | 0 |
| Morningglory | 0 | 0 | 0 | 65 | 0 | 0 | 10 | 0 |
| Nightshade | 0 | 45 | 65 | 60 | 0 | 0 | 50 | 60 |
| Nutsedge, Yellow | 0 | 0 | 20 | 0 | 0 | 0 | 20 | 10 |
| Oat, Wild | 0 | 45 | 0 | 10 | 20 | 0 | 30 | 0 |
| Pigweed | 0 | 50 | 20 | 0 | 0 | 100 | 0 | 0 |
| Ragweed | 0 | 0 | 0 | 40 | 0 | 0 | 0 | 0 |
| Russian Thistle | 0 | — | 0 | — | — | — | 60 | 0 |
| Ryegrass, Italian | 0 | 15 | 30 | 0 | 0 | 0 | 0 | 30 |
| Soybean | 0 | 0 | 0 | 0 | 0 | 0 | 0 | 0 |
| Sunflower | 0 | 0 | 15 | 0 | 0 | 0 | 0 | 0 |
| Surinam Grass | 80 | 60 | 95 | 0 | 55 | 40 | 50 | 0 |
| Velvetleaf | 0 | 0 | 0 | 0 | 0 | 0 | 0 | 0 |
| Wheat | 0 | 0 | 0 | 0 | 0 | 0 | 0 | 0 |
| | Compounds | | | | | | | |
| 16 g ai/ha | 2 | 6 | 8 | 11 | 16 | 18 | 21 | 22 |

Preemergence

| | | | | | | | | |
|---|---|---|---|---|---|---|---|---|
| Bermudagrass | 0 | 100 | 100 | 65 | — | 0 | 90 | 98 |
| Blackgrass | 0 | 50 | 0 | 0 | 10 | 0 | 40 | 0 |
| Bromegrass, Downy | 0 | 0 | 0 | 0 | 0 | 0 | 0 | 0 |
| Cocklebur | 0 | 0 | 0 | 0 | 0 | 0 | 0 | 0 |
| Corn | 0 | 0 | 10 | 0 | 0 | 0 | 0 | 0 |
| Crabgrass, Large | 65 | 95 | 95 | 65 | 70 | 0 | 20 | 85 |
| Cupgrass, Woolly | 75 | 60 | 85 | 85 | 0 | 0 | 100 | 100 |
| Foxtail, Giant | 50 | 0 | 80 | 0 | 0 | 0 | 0 | 50 |
| Foxtail, Green | 10 | 50 | 70 | 20 | 0 | 30 | 0 | 50 |
| Galium | 100 | 0 | 0 | 0 | 0 | — | 0 | 0 |
| Goosegrass | 65 | 100 | 98 | 65 | 85 | 0 | 50 | 30 |
| Johnsongrass | 10 | 45 | 75 | 0 | 0 | 15 | 0 | 0 |
| Kochia | 0 | — | — | — | 0 | 0 | 0 | 0 |
| Lambsquarters | — | 0 | 55 | 0 | 0 | 0 | 0 | 0 |
| Morningglory | 0 | 0 | 0 | 0 | 0 | 0 | 0 | 0 |
| Nightshade | 0 | 0 | 0 | 0 | 0 | 0 | 50 | 0 |
| Nutsedge, Yellow | 0 | 0 | 0 | 0 | 0 | 0 | 0 | 0 |
| Oat, Wild | 0 | 30 | 0 | 0 | 0 | 0 | 0 | 0 |
| Pigweed | 0 | 0 | 0 | 0 | — | 0 | 0 | — |
| Ragweed | — | 0 | 0 | 0 | 0 | 0 | 0 | 0 |
| Russian Thistle | 0 | — | 0 | — | — | — | 0 | — |
| Ryegrass, Italian | 0 | 0 | 0 | 0 | 0 | 0 | 0 | 0 |
| Soybean | — | 0 | 0 | 0 | 0 | 0 | 0 | 0 |
| Sunflower | 0 | 0 | 0 | 0 | 0 | 0 | 0 | 0 |
| Surinam Grass | 0 | 20 | 65 | 0 | 0 | 0 | 50 | 0 |
| Velvetleaf | 0 | 0 | 0 | 0 | 0 | 0 | 0 | 0 |
| Wheat | 0 | 0 | 0 | 0 | 0 | 0 | 0 | 0 |

| | |
|---|---|
| | Compound |
| 8 g ai/ha | 2 |

Preemergence

| | |
|---|---|
| Bermudagrass | 0 |
| Blackgrass | 0 |
| Bromegrass, Downy | 0 |
| Cocklebur | 0 |
| Corn | 0 |
| Crabgrass, Large | 0 |
| Cupgrass, Woolly | 0 |
| Foxtail, Giant | 0 |
| Foxtail, Green | 0 |
| Galium | 0 |
| Goosegrass | 0 |
| Johnsongrass | 0 |
| Kochia | 0 |
| Lambsquarters | 0 |
| Morningglory | 0 |
| Nightshade | 0 |
| Nutsedge, Yellow | 0 |
| Oat, Wild | 0 |
| Pigweed | 0 |
| Russian Thistle | 0 |
| Ryegrass, Italian | 0 |
| Soybean | 0 |
| Sunflower | 0 |
| Surinam Grass | 0 |
| Velvetleaf | 0 |
| Wheat | 0 |

### Test C

Seeds of plant species selected from annual bluegrass (Poa annua), blackgrass (Alopecurus myosuroides), canarygrass (Phalaris minor), gallium, (catchweed bedstraw, Galium aparine), chickweed (Stellaria media), downy bromegrass (Bromus tectorum), field poppy (Papaver rhoeas), field violet (Viola arvensis), green foxtail (Setaria viridis), deadnettle (henbit deadnettle, Lamium amplexicaule), Italian ryegrass (Lolium multiflorum), kochia (Kochia scoparia), lambsquarters (Chenopodium album), oilseed rape (Brassica napus), pigweed (Amaranthus retroflexus), Russian thistle (Salsola kali), spring barley (Hordeum vulgare), spring wheat (Triticum aestivum), wild buckwheat (Polygonum convolvulus), wild mustard (Sinapis arvensis), wild oat (Avena fatua), wild radish (Raphanus raphanistrum), windgrass (Apera spica-venti), winter barley (Hordeum vulgare), and winter wheat (Triticum aestivum) were planted and treated preemergence with test chemicals formulated in a non-phytotoxic solvent mixture which included a surfactant.

Treated plants and controls were maintained in a controlled growth environment for 21 days after which time all species were compared to controls and visually evaluated. Plant response ratings, summarized in Table C, are based on a scale of 0 to 100 where 0 is no effect and 100 is complete control. A dash (-) response means no test result.

TABLE C

Compound

| | |
|---|---|
| 6 | 8 |

Preemergence

| | | |
|---|---|---|
| 250 g ai/ha | | |
| Barley, Spring | 20 | |
| Barley, Winter | 20 | |
| Bluegrass | 90 | |
| Bromegrass, Downy | 35 | |
| Buckwheat, Wild | 0 | |
| Chickweed | 50 | |
| Deadnettle | 0 | |
| Field Poppy | 100 | |
| Field Violet | 0 | |
| Galium | 0 | |
| Kochia | 0 | |
| Lambsquarters | 35 | |
| Mustard, Wild | 0 | |
| Oat, Wild | 30 | |
| Oilseed Rape | 0 | |
| Pigweed | 100 | |
| Radish, Wild | 0 | |
| Ryegrass, Italian | 95 | |
| Wheat, Spring | 15 | |
| Wheat, Winter | 20 | |
| 125 g ai/ha | | |
| Barley, Spring | 20 | 0 |
| Barley, Winter | 15 | 0 |
| Blackgrass | 98 | 35 |
| Bluegrass | 20 | 0 |
| Bromegrass, Downy | 25 | 0 |
| Buckwheat, Wild | 0 | — |
| Canarygrass | 35 | 20 |
| Chickweed | 0 | 40 |
| Deadnettle | 0 | 0 |
| Field Poppy | 90 | 75 |
| Field Violet | 0 | 90 |
| Foxtail, Green | 20 | 60 |
| Galium | 0 | 0 |
| Kochia | 0 | 0 |
| Lambsquarters | 0 | 0 |
| Mustard, Wild | 0 | 100 |
| Oat, Wild | 25 | 0 |
| Oilseed Rape | 0 | 0 |
| Pigweed | 0 | 0 |
| Radish, Wild | 0 | — |
| Russian Thistle | 0 | 0 |
| Ryegrass, Italian | 15 | 0 |
| Wheat, Spring | 0 | 0 |
| Wheat, Winter | 0 | 0 |
| Windgrass | 20 | 90 |
| 62 g ai/ha | | |
| Barley, Spring | 20 | 0 |
| Barley, Winter | 0 | 0 |
| Blackgrass | — | 20 |
| Bluegrass | 20 | 0 |
| Bromegrass, Downy | 0 | 0 |
| Buckwheat, Wild | 0 | 0 |
| Canarygrass | — | 0 |
| Chickweed | 0 | 0 |
| Deadnettle | 0 | 0 |
| Field Poppy | 50 | 50 |
| Field Violet | 0 | 0 |
| Foxtail, Green | — | 15 |
| Galium | 0 | 0 |
| Kochia | 0 | 0 |
| Lambsquarters | 0 | 0 |
| Mustard, Wild | 0 | 30 |
| Oat, Wild | 25 | 0 |
| Oilseed Rape | 0 | 0 |
| Pigweed | 0 | 0 |
| Radish, Wild | 0 | — |
| Russian Thistle | 0 | 0 |
| Ryegrass, Italian | 15 | 0 |
| Wheat, Spring | 0 | 0 |
| Wheat, Winter | 0 | 0 |
| Windgrass | — | 35 |
| 31 g ai/ha | | |
| Barley, Spring | 20 | 0 |
| Barley, Winter | 0 | 0 |
| Blackgrass | — | 0 |
| Bluegrass | 20 | 0 |
| Bromegrass, Downy | 0 | 0 |
| Buckwheat, Wild | 0 | 0 |
| Canarygrass | — | 0 |
| Chickweed | 0 | 0 |
| Deadnettle | 0 | 0 |
| Field Poppy | 40 | 0 |
| Field Violet | 0 | 0 |
| Foxtail, Green | — | 0 |
| Galium | 0 | 0 |
| Kochia | 0 | 0 |
| Lambsquarters | 0 | 0 |
| Mustard, Wild | 0 | 0 |
| Oat, Wild | 20 | 0 |
| Oilseed Rape | 0 | 0 |
| Pigweed | 0 | 0 |
| Radish, Wild | 0 | — |
| Russian Thistle | 0 | 0 |
| Ryegrass, Italian | 0 | 0 |
| Wheat, Spring | 0 | 0 |
| Wheat, Winter | 0 | 0 |
| Windgrass | — | 0 |
| 16 g ai/ha | | |
| Barley, Spring | | 0 |
| Barley, Winter | | 0 |
| Blackgrass | | 0 |
| Bluegrass | | 0 |
| Bromegrass, Downy | | 0 |
| Buckwheat, Wild | | 0 |
| Canarygrass | | 0 |
| Chickweed | | 0 |
| Deadnettle | | 0 |
| Field Poppy | | 0 |
| Field Violet | | 0 |
| Foxtail, Green | | 0 |
| Galium | | 0 |
| Kochia | | 0 |
| Lambsquarters | | 0 |
| Mustard, Wild | | 0 |
| Oat, Wild | | 0 |
| Oilseed Rape | | 0 |
| Pigweed | | 0 |
| Russian Thistle | | 0 |
| Ryegrass, Italian | | 0 |
| Wheat, Spring | | 0 |
| Wheat, Winter | | 0 |
| Windgrass | | 0 |

What is claimed is:

1. A compound selected from Formula 1, N-oxides, and salts thereof, 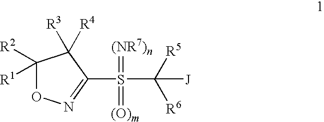
wherein
J is 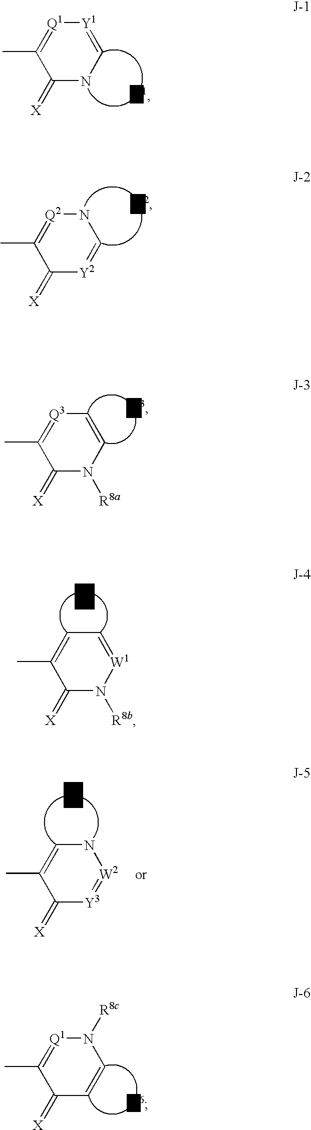
Q¹ is N or CR^{9a};
Q² is N or CR^{9b};
Q³ is N or CR^{9c};
Q⁴ is N or CR^{9d};
W¹ is N or CR^{10a};
W² is N or CR^{10b};
Y¹ is N or CR^{11a};
Y² is N or CR^{11b};
Y³ is N or CR^{11c};
X is O or S;
each G¹, G², G³, G⁴, G⁵ and G⁶ taken together with the two ring fusion atoms to which each is bonded, independently forms a fused 5-, 6- or 7-membered ring containing ring members selected from carbon atoms and up to 4 heteroatoms independently selected from up to 2 oxygen, up to 2 sulfur and up to 3 nitrogen atoms, wherein up to 1 carbon atom ring member is selected from C(═O), and the sulfur atom ring members are independently selected from S, S(O) and S(O)₂, the fused ring optionally substituted with up to 4 substituents independently selected from R¹² on carbon atom ring members and R¹³ on nitrogen atom ring members; provided that when J is J-1, J-2, J-3 or J-6 and a sulfur atom ring member of G¹, G², G³ or G⁶, respectively, is bonded to the ring fusion atom para to the connection of J to the remainder of Formula 1, then said sulfur atom ring member is S;
R¹ and R² are independently H, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₄-C₆ alkylcycloalkyl or C₄-C₆ cycloalkylalkyl; or
R¹ and R² are taken together with the carbon to which they are bonded to form a C₃-C₆ saturated carbocyclic ring optionally substituted by C₁-C₃ alkyl, halogen or C₁-C₃ haloalkyl;
R³ is H, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₄-C₆ alkylcycloalkyl or C₄-C₆ cycloalkylalkyl, C₁-C₆ alkoxy, C₁-C₆ haloalkoxy, C₁-C₆ alkylthio, C₁-C₆ haloalkylthio, —SCN, halogen, cyano, nitro, azido, —CO₂H or C₂-C₅ alkoxycarbonyl;
R⁴ is H, C₁-C₆ alkyl, C₁-C₆ haloalkyl or halogen; or
R³ and R⁴ are taken together with the carbon to which they are bonded to form a saturated carbocyclic C₃-C₆ ring optionally substituted by C₁-C₃ alkyl, halogen or C₁-C₃ haloalkyl; or
R¹ and R⁴ are taken together with the carbons to which they are bonded to form a C₃-C₇ saturated carbocyclic ring optionally substituted by C₁-C₃ alkyl, halogen or C₁-C₃ haloalkyl;
R⁵ is H, C₁-C₂ alkyl, halogen, cyano or C₂-C₅ alkoxycarbonyl;
R⁶ is H, C₁-C₂ alkyl or halogen; or
R⁵ and R⁶ are taken together with the carbon to which they are bonded to form a C₃-C₆ saturated carbocyclic ring;
R⁷ is H, —CN, C₂-C₄ alkoxycarbonyl, C₁-C₄ alkylsulfonyl, C₂-C₄ alkylcarbonyl or C₂-C₄ haloalkylcarbonyl;
R^{8a}, R^{8b} and R^{8c} are each independently C₁-C₇ alkyl, C₂-C₇ alkenyl, C₂-C₇ alkynyl, C₃-C₇ cycloalkyl, C₁-C₇ haloalkyl, C₂-C₇ haloalkenyl, C₂-C₇ alkoxyalkyl, C₄-C₇ cycloalkylalkyl, C₃-C₇ haloalkynyl, C₃-C₇ alkylcarbonylalkyl, C₃-C₇ alkoxycarbonylalkyl, C₄-C₇ halocycloalkylalkyl, C₂-C₇ haloalkoxyalkyl, C₂-C₇ alkylthioalkyl, C₂-C₇ alkylsulfonylalkyl, C₂-C₇ alkylsulfinylalkyl, C₂-C₇ cyanoalkyl, C₂-C₇ haloalkylthioalkyl, C₂-C₇ haloalkylsulfonylalkyl, C₂-C₇ haloalkylsulfinylalkyl, C₃-C₇ haloalkoxycarbonylalkyl, C₃-C₇ haloalkylcarbonylalkyl; C₁-C₆ alkylsulfonyl, C₁-C₆ haloalkylsulfonyl or C₃-C₆ cycloalkylsulfonyl; and
R^{9a}, R^{9b}, R^{9c}, R^{9d}, R^{10a}, R^{10b}, R^{11a}, R^{11b} and R^{11c} are each independently H, halogen, cyano, C₁-C₇ alkyl, C₂-C₇ alkenyl, C₂-C₇ alkynyl, C₃-C₇ cycloalkyl, C₄-C₇ cycloalkylalkyl, C₄-C₇ alkylcycloalkyl, C₁-C₇ haloalkyl, C₂-C₇ haloalkenyl, C₃-C₇ haloalkynyl, C₃-C₇ halocycloalkyl, C₁-C₇ alkoxy, C₁-C₇ haloalkoxy, C₂-C₇ alkenyloxy, C₂-C₇ haloalkenyloxy, C₃-C₇ cycloalkoxy, C₃-C₇ halocycloalkoxy, C₂-C₇ alkynyloxy, C₄-C₇ cycloalkylalkoxy, C₄-C₇ halocycloalkylalkoxy, C₁-C₇ alkylthio, C₁-C₇ haloalkylthio, C₁-C₇ haloalkylsulfinyl, C₁-C₇ haloalkylsulfonyl, C₁-C₆ alkylamino, C₂-C₇ dialkylamino, C₁-C₇ haloalkylamino, C₂-C₇ alkylcarbonyl, C₂-C₇ alkoxycarbonyl, C₂-C₇ haloalkylcarbonyl, C₂-C₇ haloalkoxycarbonyl, C₃-C₇ alkylcarbonylalkyl, C₃-C₇, alkoxycarbonylalkyl, aminocarbonyl, C₂-C₈ alkylaminocarbonyl, C₂-C₈ haloalkylaminocarbonyl, C₃-C₈ dialkylaminocarbonyl, C₃-C₈ haloalkyl(alkyl)aminocarbonyl, C₄-C₇ cycloalkylaminocarbonyl, C₅-C₈ cycloalkyl(alkyl)aminocarbonyl, C₄-C₇ halocycloalkylalkyl, C₁-C₇ alkoxyalkyl, C₂-C₇ haloalkoxyalkyl, C₂-C₇ alkylthioalkyl, C₂-C₇ alkylsulfonylalkyl, C₂-C₇ alkylsulfinylalkyl, C₂-C₇ cyanoalkyl, C₂-C₇ haloalkylthioalkyl, C₂-C₇ haloalkylsulfonylalkyl, C₂-C₇ haloalkylsulfinylalkyl, C₃-C₇ haloalkoxycarbonylalkyl, C₃-C₇ haloalkylcarbonylalkyl, C₂-C₇ alkoxyalkoxy, C₂-C₇ haloalkoxyalkoxy, C₂-C₇ alkylthioalkoxy, C₂-C₇ haloalkylthioalkoxy, C₂-C₇ haloalkylsulfonylalkoxy, C₂-C₇ haloalkylsulfinylalkoxy, nitro, C₃-C₁₀ trialkylsilyl, aminosulfonyl, C₁-C₇ alkylaminosulfonyl, C₁-C₇ haloalkylaminosulfonyl, C₂-C₈ dialkylaminosulfonyl, C₃-C₈ haloalkyl(alkyl)aminosulfonyl, C₃-C₆ cycloalkylaminosulfonyl or C₄-C₇ cycloalkyl(alkyl)aminosulfonyl;
each R¹² is independently C₁-C₆ alkyl, C₁-C₆ haloalkyl, C₁-C₆ alkoxy, cyano or halogen;
each R¹³ is independently C₁-C₆ alkyl or C₁-C₆ haloalkyl;
m is 0, 1 or 2; and
n is 0 or 1; provided that the sum of n and m is not more than 2.

2. A herbicidal composition comprising a compound of claim 1 wherein the sum of n and m is 1 or 2, and at least one component selected from the group consisting of surfactants, solid diluents and liquid diluents.

3. A herbicidal composition comprising a compound of claim 1 wherein the sum of n and m is 1 or 2, at least one additional active ingredient selected from the group consisting of other herbicides and herbicide safeners, and at least one component selected from the group consisting of surfactants, solid diluents and liquid diluents.

4. A method for controlling the growth of undesired vegetation comprising contacting the vegetation or its environment with a herbicidally effective amount of a compound of claim 1 wherein the sum of n and m is 1 or 2.
